(19) United States
(12) Patent Application Publication
(10) Pub. No.: US 2018/0057888 A1
(43) Pub. Date: Mar. 1, 2018

(54) KUB5/HERA AS A DETERMINANT OF SENSITIVITY TO DNA DAMAGE

(71) Applicant: The Board of Regents of the University of Texas System, Austin, TX (US)

(72) Inventors: David A. BOOTHMAN, Dallas, TX (US); Edward A. MOTEA, Dallas, TX (US); Praveen PATIDAR, Dallas, TX (US); Julio MORALES, Dallas, TX (US)

(21) Appl. No.: 15/557,608

(22) PCT Filed: Mar. 8, 2016
(86) PCT No.: PCT/US16/21311

§ 371 Date: Sep. 12, 2017

Related U.S. Application Data
(60) Provisional application No. 62/132,985, filed on Mar. 13, 2015.

(51) Int. Cl. C12Q 1/68; C12N 15/113; A61N 5/10

(52) U.S. Cl. CPC C12Q 1/6886; C12N 15/1137; C12Y 204/0203; A61N 5/10; C12N 2310/531; C12Q 2600/158; C12Q 2600/118; C12Q 2600/106; C12N 2310/14; C12Q 2600/156

(57) ABSTRACT

The present disclosure is directed to methods of detecting KUB5/HERA expression levels, copy number and mutation status, particularly in cancer cells. The methods permit physicians to tailor therapies to subject having certain genotypes/phenotypes, and to exclude therapies unlikely to be effective.

This application claims benefit of priority to U.S. Provisional Application Ser. No. 62/132,985, filed Mar. 13, 2015, the entire contents of which are hereby incorporated by reference.

### BACKGROUND

1. Field of the Disclosure

The present disclosure relates generally to the fields of medicine, oncology, and molecular biology. More particular, the disclosure relates to detecting Kub5/Hera expression and structural alterations to guide cancer therapy and prognosis.

### 2. Background

The integrity of genetic information stored in the genome of each cell is continuously challenged by exposure to a myriad of genotoxic environmental changes. As such, optimal growth and survival of normal cells depend on a full spectrum of functional DNA damage checkpoints and DNA repair pathways to maintain genomic integrity. In cancer cells, however, only some of these mechanisms remain intact and operational, which often serve as their main lifeline for survival. Indeed, targeting these aberrant ‘cancer vulnerable’ pathways that commonly occur only in cancer cells represents potential druggable targets for selective destruction of malignant cells (Lord and Ashworth, 2012 and Jackson and Bartek, 2009). Poly(ADP-ribose) polymerase 1 (PARP1) is a major DNA damage sensor that detects DNA lesions arising endogenously (i.e., generation of oxidative DNA lesions) or exogenously (i.e., exposure to cytotoxic chemotherapeutic agents), and then stimulates immediate DNA repair or cell cycle arrest checkpoint pathways (Schreiber et al., 2006 and Plummer, 2006). Deficits in PARP1 activity cause excessive accumulation of DNA single-strand breaks (SSBs) that initiate replication fork collapse and formation of DNA double-strand breaks (DSBs) upon collision with replication forks (Helleday, 2001). DSBs created during replication are preferentially repaired by homologous recombination (HR) to maintain genomic integrity (Hoeijmakers, 2001). One unrepaired DSB in eukaryotic cells is sufficient to cause lethality. One of the major components of HR is BRCA1 (breast cancer associated gene 1), which upon phosphorylation by cyclin-dependent kinase 1 (CDK1), is recruited to sites of DNA damage to facilitate repair and checkpoint activation (Johnson et al., 2009). However, it remains unknown how aberrant up-regulation of CDK1 transcription is regulated in tumors to mediate DSB repair. Failure to activate HR engages PARP-dependent alternative repair pathway to salvage the genotoxic effect of a persistent DSB (Bryant et al., 2005 and Farmer et al., 2005).

Deficiencies in BRCA1 and BRCA2 genes can compromise HR-mediated repair, conferring hypersensitivity to poly(ADP-ribose) polymerase-1 (PARP1) inhibitors (PARP1i) by synthetic lethality (Bryant et al., 2005; Farmer et al., 2005 and Ashworth, 2008). The rarity of adult tumors with ‘BRCAness’ (i.e., BRCA-deficiency), however, currently restricts the therapeutic utility of PARP inhibitor monotherapy, despite the enthusiasm evoked by promising clinical trials (Fong et al., 2009; Audeh et al., 2010 and Tutt et al., 2010). This is further complicated by several reports of potential molecular mechanisms of resistance for PARP1i in BRCA-deficient cancers (Michalak and Jonkers, 2011). One striking enigma, though, is that ˜24% of high-grade triple negative breast cancers (TNBC) without a BRCA mutation responded positively to PARP1 inhibitors in Phase II clinical trials (Tutt et al., 2010). These confounding observations clearly point towards a search for suitable predictive markers and novel therapeutic targets that can potentiate the DNA damaging effects of PARP1 inhibitors beyond BRCA deficiencies.

### SUMMARY

Thus, in accordance with the present disclosure, there is provided a method of treating a cancer patient determined to (a) express low/undetectable levels of Kub-5-Hera (K-H), (b) have reduced K-H gene copy number relative to a normal cell, and/or (c) have a loss/reduction of function mutation in a K-H coding region, such as mutation in a K-H RPR (CID) domain, and/or mutation in K-H coiled-coil domain, comprising administering to said subject a PARP1 inhibitor, ionizing radiation, or a chemotherapeutic agent that induces DNA double-strand breaks. The method may further provide for the determination of (a) low/undetectable levels of K-H, (b) reduced K-H gene copy number and/or (c) mutation in a K-H coding region. The chemotherapeutic agent that induces DNA double-strand breaks may be doxorubicin, a Topoisomerase I or II poison, paclitaxel, cisplatin, or gemcitabine. The PARP1 inhibitor may be selected from rucaparib or olaparib. The cancer is selected from breast cancer, lung cancer, pancreatic cancer, brain cancer, ovarian cancer, head and neck cancer and cervical cancer, and the breast cancer may be a BRCA-proficient breast or pancreatic cancer. Copy number or expression level may be determined relative to housekeeping genes such as β-actin and GAPDH, for which copy number and expression do not change, or other proteins, levels of which do not change, such as MLH1 and MSH2.

Analysis may comprise expression analysis, such as quantitative mRNA level analysis, including PCR, Northern blot analysis or in situ hybridization. Expression analysis may also comprises protein analysis, such as protein analysis comprises ELISA, immunohistochemistry or LC-MS/MS. Analysis may comprise structural analysis, such as copy number variation analysis, FISH analysis or SNP analysis. The method may further comprise screening for mutated or low/undetectable levels of CDK1, and/or for mutated or low/undetectable levels of Artemis. The patient may be treated only with PARP1 inhibitor, may be treated only with ionizing radiation, or may be treated with PARP1 inhibitor and ionizing radiation. The patient may be a human patient, or a non-human mammalian patient.

In another embodiment, there is provided a method of predicting a cancer patient's response to cancer therapy comprising determining expression of Kub-5-Hera (K-H), K-H gene copy number, mutation in a K-H RPR (CID) domain, and/or mutation in K-H coiled-coil domain in cancer cells from said patient, wherein (a) overexpression or increased copy number of K-H confers resistance to PARP1 inhibition, radiation therapy, and chemotherapies that induce double-strand breaks; (b) under-expression of K-H, reduced copy number of K-H, or loss/reduction of function mutation in a K-H RPR (CID) domain, and/or loss/reduction of function mutation in K-H coiled-coil domain in cancer cells from said patient confers sensitivity to PARP1 inhibition, radiation therapy, and chemotherapies that induce double-strand breaks. The method may further comprise determining expression levels and/or the presence or absence of mutation in CDK1 in said patient, wherein reduced expression, decreased copy number or mutation of CDK1 confers hypersensitivity to PARP1 inhibition, radiation therapy and chemotherapies that induce double-strand breaks. The method may further comprise determining expression levels and/or the presence or absence of mutation in Artemis, wherein reduced expression or mutation of Artemis confers hypersensitivity to radiation therapy and chemotherapies that induce double-strand breaks. Analysis may comprise expression analysis, such as quantitative mRNA level analysis, including PCR, Northern blot analysis or in situ hybridization. Expression analysis may also comprises protein analysis, such as protein analysis comprises ELISA, immunohistochemistry or LC-MS/MS. Analysis may comprise structural analysis, such as copy number variation analysis, FISH analysis or SNP analysis.

In still another embodiment, there is provided a method of predicting a subject's carcinogenic risk comprising determining expression of low/undetectable levels of Kub-5-Hera (K-H), reduced K-H gene copy number, loss/reduction of function mutation in a K-H RPR (CID) domain, and/or loss/reduction of function mutation in K-H coiled-coil domain, wherein under-expression, reduced gene copy number, or loss/reduction of function mutation increases risk of carcinogenesis from environmental carcinogens or disease that cause inflammation. The method may further comprise determining expression levels, decreased copy number and/or the presence or absence of mutation in CDK1, wherein low levels or mutation of CDK1 increases risk of carcinogenesis from environmental carcinogens or disease that cause inflammation. The method may further comprising determining expression levels and/or the presence or absence of mutation in Artemis, wherein low levels or mutation of Artemis increases risk of carcinogenesis from environmental carcinogens or disease that cause inflammation. Analysis may comprise expression analysis, such as quantitative mRNA level analysis, including PCR, Northern blot analysis or in situ hybridization. Expression analysis may also comprise protein analysis, such as protein analysis comprises ELISA, immunohistochemistry or LC-MS/MS. Analysis may comprise structural analysis, such as copy number variation analysis, FISH analysis or SNP analysis. The environmental carcinogen may be low grade radiation, diagnostic x-rays, therapeutic radiation, ablative radiation or an inflammatory disease such as colonitis or pancreatitis.

In yet a further embodiment, there is provided a method of predicting a subject's metastatic potential comprising determining elevated expression of Kub-5-Hera (K-H), or increased K-H gene copy number, where (i) overexpression or increased copy number leads to growth changes and enhanced metastic spreading potential. Analysis may comprise expression analysis, such as quantitative mRNA level analysis, including PCR, Northern blot analysis or in situ hybridization. Expression analysis may also comprises protein analysis, such as protein analysis comprises ELISA, immunohistochemistry or LC-MS/MS. Analysis may comprise structural analysis, such as copy number variation analysis, FISH analysis or SNP analysis.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-I. Kub5-Hera (K-H) depletion is synthetic lethal with PARP1 inhibition. (FIG. 1A) Relative PARP enzymatic activity [PAR formation/Time (sec)] for parental and stable K-H (shK-H), PARP1 (shPARP1) or Scrambled (shSCR) knockdown MDA-MB-MDA 231 triple-negative breast cancer (TNBC) cells. All activities were inhibited by Rucaparib, a PARP1 inhibitor. (FIG. 1B) Clonogenic survival of parental and stable shRNA knockdown 231 cells in FIG. 1A, treated with or without Rucaparib. Data points are % means±SEM from three experiments, ***p<0.001. (FIG. 1C) Cell death (% Apoptosis) assessment of 231 cells described in FIG. 1B by TUNEL+ staining after DMSO or Rucaparib treatments. (FIG. 1D) Western blot analyses of shSCR or shK-H 231 cells transiently transfected with small interfering RNA to nonsense (Scramble (SCR)) siSCR or PARP1 si-PARP1. (FIG. 1E) Clonogenic survival of 231 cells treated in FIG. 1D. (FIG. 1F) Western blot analyses of stable SCR (shSCR) or PARP1 (shPARP1) sequences in 231 cells transiently transfected with siSCR or siK-H. (FIG. 1G) Clonogenic Survival of 231 cells treated in FIG. 1F. (FIG. 1H) Western blot analyses of shPARP1 or shSCR 231 cells transiently transfected with siSCR or siK-H at indicated times. (FIG. 1I) Apoptosis (% TUNEL+ staining) from cells in FIG. 1H. Data are % means±SEM from three experiments; *p<0.05; **p<0.01; ***p<0.001.

FIGS. 2A-C. Sensitivity of shK-H-depleted cells to Rucaparib is accompanied by persistent DSBs. (FIG. 2A) Representative images for γ-H2AX and 53BP1 foci formation, surrogate DSB markers, in shSCR or shK-H 231 cells after vehicle (0.01% DMSO) or Rucaparib treatment for 24 hr. DAPI stained cell nuclei. Scale bars: 30 μm. (FIG. 2B) Rate of DSB formation in shSCR or shK-H 231 cells after Vehicle or Rucaparib exposure for 24 hr. Data are means 53BP1 foci/nuclei for ≧50 cells; bars, ±SEM. (FIG. 2C) Persistent DSB (co-localized γ-H2AX and 53BP1 foci/nuclei) formation in shSCR or shK-H cells after Vehicle or Rucaparib treatment for 24 hr. Data are % mean of nuclei with ≧5 co-localized γ-H2AX/53BP1 foci, ±SEM from three independent experiments.

FIGS. 3A-D. K-H loss compromises CDK1 expression/activity loss and reduced homologous recombination (HR)-mediated DSB repair. (FIG. 3A) shSCR 231 cells were treated with siK-H and whole cell lysates were analyzed by Western blotting. Abbreviations: K-H, Kub5-Hera; CDK1, cyclin-dependent kinase 1; CDK2, cyclin-dependent kinase 2; pS1497 BRCA1, phosphorylated serine 1497 of BRCA1; pS139 H2AX, phosphorylated serine 139 of H2AX. (FIGS. 3B-C) Western blot analyses of HCC1569 (FIG. 3B) or Parental 231 (FIG. 3C) breast cancer cells treated with siSCR or siK-H. (FIG. 3D) Homologous recombination (HR) assays of HEK293 cells treated with siSCR, siRad51, siCDK1 or siK-H transfected or not with CDK1 cDNA using the DR-GFP reporter system. Data are % means GFP+ cells normalized to siSCR, ±% SEM from three independent experiments; *p<0.05, ns=not significant.

FIGS. 4A-F. K-H loss as a potential biomarker for PARP1 inhibitor lethality in BRCA-proficient breast cancers. (FIG. 4A) CGH array analyses for k-h gene copy number variation in a panel of breast cancer cells. (FIG. 4B) k-h mRNA expression vs CGH. p=0.0089, R=0.88. (FIG. 4C) k-h mRNA expression vs protein levels in a subset of breast cancer cells selected based on gain or loss of k-h copy number. p=0.007, R=0.89. (FIG. 4D) Western blot analysis of K-H, CDK1, and PARP1 protein levels in various breast cancer cells. Sensitivities to Rucaparib are summarized in Table S1. (FIG. 4E) Rucaparib LC₅₀ values vs relative K-H protein levels in BRCA-proficient breast cancer cell lines; p=0.008, R=0.81. (FIG. 4F) Rucaparib LC₅₀ values vs relative CDK1 protein levels in BRCA-proficient breast cancer cell lines; p=0.038, R=0.69. See Supplementary Table S1 for additional information.

FIGS. 5A-D. K-H promotes transcription by recruiting RNAPII to the CDK1 promoter. (FIGS. 5A-B) k-h and cdk1 mRNA expression levels in: 231 (FIG. 5A) or HCC1569 (FIG. 5B) cells after siSCR or siK-H (3′-UTR) treatments. (FIG. 5C) k-h and cdk1 mRNA expression levels in stable shSCR or shK-H 231 cells. Data are means±SEM from three independent experiments. (FIG. 5D) Top. Western blot analyses of K-H and CDK1 levels in shSCR or shK-H (ORF) 231 cells. Lamin B, loading control for nuclear protein extracts. Bottom, ChIP assessment of RNAPII recruitment to CDK1 or actin promoter regions in shSCR vs shK-H 231 cells. Primers adjacent to indicated gene promoter are listed in Supplementary Table S5. Data are means±SEM from three separate experiments. Statistics: ***p<0.001, **p<0.01, ns=not significant.

FIGS. 6A-G. K-H residue R106 is essential for RNAPII CTD (pS2) binding to promote CDK1 transcription via recruitment to the CHR promoter element. (FIG. 6A) Structural representation of K-H bound to the pS2 CTD tail of RNAPII. Binding is by hydrogen bonding and electrostatic interactions between negatively-charged p-Ser2 of RNAPII and positively-charged Arg106. (FIG. 6B) IP pull-down of RNAPII by myc-tagged wild-type K-H protein. (FIG. 6C) Myc-tagged K-H R106A mutant IP shows reduced pull-down of RNAPII due to loss of crucial interactions. (FIG. 6D) CDK1 protein level rescue by wild-type K-H, but not K-H R106A mutant, re-expression in 3′-UTR siK-H knockdown 231 cells. Lamin B was used as a loading control for nuclear protein extracts. (FIGS. 6E-F) CDK1-dependent reporter activity assays in: shSCR 231 (FIG. 6E) or HCC1569 (FIG. 6F) BRCA1-breast cancer cells treated with siSCR or siK-H, or transfected with vector alone CMV-K-H wild-type or K-H R106A mutant cDNAs. (FIG. 6G) Right, Sequence of wild-type and mutant forms of CDE and CHR CDK1 promoter elements (WT=SEQ ID NO: 1; M1=ID NO: 2; M2=SEQ ID NO: 3; M3=SEQ ID NO: 4; M4=SEQ ID NO: 5). Left, Relative CKD1 promoter activity for wild-type and mutant CDK1 promoter elements after K-H over-expression. Data are means±SEM from three experiments; *p<0.05; **p<0.01; ***p<0.001, ns=not significant.

FIGS. 7A-C. Wild-type, but not mutant R106A K-H, rescues CDK1 expression, BRCA1 phosphorylation and reverses Rucaparib lethality. (FIG. 7A) Western blot analyses of CDK1, total and pS1497 BRCA1 protein levels in shK-H 231 cells after forced ectopic expression of mock-transfected, empty pCMV vector or wild-type vs mutant R106A K-H. (FIG. 7B) Top, Clonogenic survival shK-H 231 cells treated in FIG. 7A. Bottom, representative plates of surviving colonies after treatment with DMSO or Rucaparib (5 μM. Data are means±SEM; *p<0.05, **p<0.01, ***p<0.001, ns=not significant. (FIG. 7C) Model depicting K-H regulation of CDK1 expression, BRCA1 phosphorylation and HR activity highlighting the mechanism of synthetic lethality in K-H-deficient cells following PARP1 inhibition.

FIGS. 8A-H. Pharmacological and genetic ablation of PARP1 activity is synthetic lethal with aberrant K-H reduction in BRCA-proficient breast cancers. (FIG. 8A) Western blot analysis of the parental BRCA-proficient 231 TNBC compared with 231 cells engineered to generate stable short hairpin RNA targeting non-specific gene control (shSCR), Kub5-Hera (shK-H) or PARP1 (shPARP1). (FIG. 8B) Chemical structures of PARP inhibitors used in this study. Left, AG014361; Right, AG014699 or Rucaparib. (C) Colony formation of specified cell lines treated with varying concentrations (0, 1, 2.5, 5, 10, 25 μM) of PARP inhibitor, AG014361, for 24 hr. Survival is expressed as mean percentage of colonies formed relative to vehicle-treated (0.01% DMSO) cells; error bars represent ±s.e.m. from three replicates. (FIG. 8D) Total cellular proteins from indicated 231 cells treated with vehicle (0.01% DMSO), AG014699/Rucaparib (5 μM), or staurosporine (STS, 2 μM) were subjected to Western blot analysis with indicated antibodies. (FIG. 8E) Western blot analysis of HCC1937 cell lines stably transfected with empty vector (lane 1), BRCA1 cDNA (lane 2), BRCA1 cDNA+ transient siSCR (lane 3), or BRCA1 cDNA+ transient siK-H (lane 4). Cells in FIG. 8E were subjected to Rucaparib (30 μM) treatment for 24 hr, then allowed to grow in drug-free media until confluent. Since HCC1937 cells do not perform robust colony formation ability, viability was measured using Sulforhodamine B Assay as previously described¹. Colorimetric intensity values were obtained using a plate reader, and normalized to control (0.01% DMSO). (FIG. 8F) Comparison of growth inhibition after Rucaparib treatment in [HCC1937+empty vector] versus [HC1937+BRCA1 cDNA]. Rucaparib treatment is described above. Data are % means±SEM from three independent experiments repeated twice; ***p<0.001. (FIG. 8G) Comparison of growth inhibition after Rucaparib treatment in [HC1937+BRCA1 cDNA] cells transiently transfected with siSCR versus siK-H (15 nM, 48 hr). Rucaparib treatment is described above. Data are % means±SEM from three independent experiments repeated twice; ***p<0.001. (FIG. 8H) Comparison of growth inhibition after Rucaparib treatment in 231 TNBC cells transiently transfected with siSCR versus siK-H (15 nM, 48 hr). Rucaparib treatment is described above. Data are % means±SEM from three independent experiments repeated twice; ***p<0.001.

FIGS. 9A-H. Loss of K-H compromises CDK1 expression/activity and reduces HR-mediated DSB repair. (FIG. 9A) 231 shPARP1 cells transiently transfected with indicated concentrations of siK-H for 48 hr. Total cellular proteins from each treatments were subjected to Western blot analysis using indicated antibodies. (FIG. 9B) Western blot analysis of CDK1 protein levels in 231 shSCR cells treated with siK-H (15 nM) and harvested at various times (24, 48, 72 hr). (FIG. 9C) Western blot analysis of CDK1 protein levels in 231 cells treated with siRNA to nonspecific gene control (siSCR) or specific genes as indicated (15 nM). Specific siRNA oligonucleotide sequences used in this study are listed in Table S3. (FIG. 9D) Asynchronous 231 shSCR versus shK-H cells pulsed with 5-ethynyl-2′-deoxy-Uridine (EdU, 10 μM, 1 hr) at log phase. (FIG. 9E) 231 cells treated with ionizing radiation (IR, 2 Gy) and harvested after 1 hr. Top, The blot shows reduction of CDK1 in shK-H compared to shSCR, but no further reduction of CDK1 protein level was observed after IR treatment. Bottom, Endogenous phospho-S1497 BRCA1 is higher in shSCR compared to shK-H. After IR treatment, S1497 BRCA1 phosphorylation is impaired in 231 shK-H but not in shSCR. (FIG. 9F) 231 cells treated with PARP1i Rucaparib (5 μM, 24 hr), CDK1 inhibitor RO3066 (0.5 and 1 μM, 24 hr) or combination [Rucaparib+RO3066]. Cells were harvested after 24 hr treatment. The immunoblot shows no further reduction of CDK1 after Rucaparib and/or RO3066 treatment. BRCA1 was activated (pS1497-BRCA1) after Rucaparib treatment alone (lane 2) but not in combination with CDK1 inhibitor, RO3066 (lanes 5 and 6). Compromising CDK1 activity has been shown to inhibit BRCA1 phosphorylation at S1497 site, and impairs HR-mediated DSB repair (Johnson et al., 2009 and Johnson et al., 2011). (FIG. 90) Comparison of 231 cells (shSCR versus shK-H) treated with Rucaparib (5 μM, 24 hr), CDK1 inhibitor RO3066 (1 μM, 24 hr) or combination [Rucaparib+RO3066]. Cells were harvested after 24 hr treatment. The blot shows reduction of CDK1 protein level in shK-H (lanes 6-8), but not in shSCR cells (lanes 1-4). No further reduction of CDK1 protein level was seen after Rucaparib and/or RO3066 treatments. Endogenous pS1497-BRCA1 was higher in vehicle-treated shSCR (lane 1) compared to shK-H (lane 6). Treatment with Rucaparib engaged more pS1497-BRCA1 activation in shSCR cells (lane 2), but was inhibited in combination with CDK1 inhibitor treatment (lane 4) (results similar to FIG. 9F). 231 shSCR cells treated with both Rucaparib and RO3066 CDK1 inhibitor exhibited comparable pS1497-BRCA1 activation as 231 shK-H cells treated with Rucaparib only where CDK1 expression/activity is already compromised (compare lane 4 vs. 7). (FIG. 9H) Homologous recombination assay in MCF-7 cells using DR-GFP reporter system. Columns represent mean percentage of GFP-positive cells normalized to control (siSCR); error bars represent ±s.e.m. from duplicate experiments, *p<0.05, **p<0.01. See Table S3 for siRNA sequences. siK-H used was to 3′-UTR.

FIGS. 10A-B. Distribution of k-h Copy Number Alteration (CNA) and mRNA expression in breast cancer patients derived from The Cancer Genome Atlas (TCGA). (FIG. 10A) Waterfall plot demonstrating the distribution of copy number alterations in breast cancer TCGA; (+) represents amplification and (−) represents deletion of k-h copy number. Approximately 18% of breast cancer patients show deletion of k-h copy number. (FIG. 10B) Correlation between k-h mRNA expression versus CNA in breast cancer TCGA data, n=1057 patient samples. The results shown here are in whole or part based upon data generated by the TCGA Research network (cancergenome.nih.gov). These results closely resemble the data obtained in a panel of breast cancer cell lines used in this study.

FIGS. 11A-D. K-H promotes CDK1 transcription. (FIG. 11A) Re-expression of si-resistant myc₆-tagged K-H cDNA in 231 shSCR versus shK-H (shRNA was specific to K-H coding region). (FIG. 11B) Right, Sequence of wild-type and mutant forms of CDE and CHR CDK1 promoter elements (WT=SEQ ID NO: 1; M1=ID NO: 2; M2=SEQ ID NO: 3; M3=SEQ ID NO: 4; M4=SEQ ID NO: 5). Left, Relative luciferase activity reporter assays performed in 231 shSCR cells. Depletion of K-H reduced luciferase signal, and thus CDK1 transcription. Relative CDK1 promoter activity was further reduced with mutations in CHR, but not in CDE, sequence motif. Columns represent mean luciferase signal relative to siSCR control; error bars represent±s.e.m. from three replicates, *p<0.05, ***p<0.001, ns=not significant. (FIG. 11C) Simplified model depicting the recruitment of wild-type K-H homodimer, bound to RNAPII pS2-CTD heptapeptide repeats, to the CHR region of CDK1 promoter with the putative transcription complex. (FIG. 11D) Mutation of R106 to alanine (K-H R106A) abrogates pS2-CTD RNAPII binding, which consequently impaired CDK1 transcription.

FIGS. 12B-C. Structure-function analyses of K-H and p15RS molecular modeled and tumor-derived mutations, including SNPs from breast and NSCLC cancers. (FIGS. 12B-C) Cartoons of K-H (FIG. 12B) or p15RS (FIG. 12C) show mutationns found in tumor SNPs, including breast and NSCLC cancers selected to delineate effects of abrogating K-H or p15RS interactions with RNAPII, Artemis, Ku70 and each other (heterodimers) or themselves (homodimers). Lines below indicate relative interaction domains at N-ter (RNAPII or Artemis) and C-ter (Ku70, K-H or p15RS). Note similarities between K-H and p15RS.

FIG. 13. K-H loss compromises homologous recombination (HR)-mediated DSB repair. K-H deficiency transcriptome signature obtained using Ingenuity's Pathway Analysis (IPA) of all mRNAs altered at least 2-fold after K-H depletion relative to control. ^{§}Top five resulting pathways were similar to the top five transcriptome molecular and cellular pathway signatures obtained for loss of BRCA1, RAD51, or BRIT1—termed HR Deficiency (HRD) Gene Signature (Peng et al., 2014).

FIGS. 14A-B. K-H loss compromises RAD51 recruitment to sites of double-strand breaks (DSBs) after treatment with PARP inhibitor. (FIG. 14A) Representative images for RAD51 and γ-H2AX foci formation, surrogate DSB markers, in stable shK-H or shSCR 231 cells after Rucaparib or vehicle (0.01% DMSO) treatment for 24 h. DAPI stained cell nuclei. Scale bars: 10 μm. (FIG. 14B) Quantification of cells (>50 nucleus) with ≧10 co-localized RAD51 and γ-H2AX foci formation following treatment with Rucaparib (5 μM, 24 h). bars, ±SEM, **p<0.01

FIG. 15. Kub5-Hera (K-H) deficient 231 cells are synthetic lethal to BMN-673 (Talazoparib) PARP inhbitor. Inset: Western blot analysis to confirm K-H depletion in shK-H 231 cells relative to shSCR control. Clonogenic survival plot of stable shRNA K-H knockdown versus shSCR control MDA-MB-231 cells treated with BMN673 (Talazoparib, 24 h) or DMSO vehicle control.

### DESCRIPTION OF ILLUSTRATIVE EMBODIMENTS

Here, the inventors present an innovative strategy to expand the use of PARP1 inhibitors in BRCA-proficient cancers with deficient Kub5-Hera (K-H, also known as RPRD1B, CREPT, C20orf77), the human homolog of yeast RT103 (Bhatia et al., 2014 and Becherel et al., 2013), as well as guide other therapies that rely on the induction of double-strand breaks. They show that this transcription termination factor plays a novel role in promoting the synthesis of CDK1 transcript and protein expression to mediate DSB repair. Functionally, the inventors demonstrate that loss of K-H engages a surge in PARP1 activity and promotes a state of “BRCAness” in BRCA-proficient cancer cells by compromising the transcription and activity of CDK1, a crucial component of the HR pathway that phosphorylates BRCA1 (pS1497) for proper HR function (Kim and Jinks-Robertson, 2012). At the molecular level, they show that K-H binds the phospho-Serine 2-containing C-terminal domain (CTD) of RNA Pol II (pS2-CTD RNAPII), which is then recruited to the cell-cycle homology region (CHR) of the CDK1 promoter to efficiently stimulate CDK1 transcription. Based on the mechanistic insights gained from these studies, the inventors discuss exploitable properties of K-H copy number variation and expression as a clinically relevant biomarker of PARP1i lethality in BRCA-proficient cancers. These and other aspects of the disclosure are described in greater detail below.

### I. KUB5-HERA AND RELATED PROTEINS

A. Kub5/Hera

Ku70 binding protein #5/Hera (KUB5/HERA) was identified by a yeast two-hybrid screen using Ku70 as bait. Loss of RTT103, a putative yeast homolog of KUB5/HERA, resulted in increased sensitivity to IR, similar to that observed in hdf1-deletion yeast. the yeast homolog of Ku70 Results also show that RTT103-deletion yeast are deficient in repairing blunt and non-compatible DNA ends and re-expression of hKub5/Hera can correct the IR-sensitivity and DNA repair deficiency of these deficient yeast demonstrating a strong functional model for human KUB5/HERA function in yeast. Analyses of breast cancer cell lines for their KUB5/HERA protein expression yielded a strong correlation between KUB5/HERA protein level and sensitivity to DNA damage. These data strongly suggest that KUB5/HERA is a novel repair factor involved in NHEJ and endogenous over-expression of KUB5/HERA plays a role in chemotherapeutic and/or radio-therapeutic resistance via increasing the capacity to facilitate NHEJ repair of DSBs in breast cancer cells.

Specific Roles of Kub5/Hera (K-H) in Coupled Processes of RNA Transcription Termination & Complex DSB Repair.

K-H is the human homolog of the yeast RTT103 yeast gene and is a vital scaffolding protein in RNA transcription termination. In the initial grant period, we showed that shK-H loss or depletion in yeast, mouse or human cells resulted in R-loop formation and formation of complex DSBs. When RNAPII stalls after production of capped polyA-mRNA and reaching a termination sequence, its C-terminal domain becomes phosphorylated at repeat serine 2 sequences (P-Ser2). K-H binds P-Ser2 residues, acting as a scaffolding to assemble p54nrb-PSF-pcf11-p15RS-XRN2 protein complexes to dislodge RNAPII and mediate XRN2-dependent degradation of the RNA component of the transient R-loop. Depleting K-H levels dissociated this termination complex and led to elevated persistent R-loops and basal DSBs (Morales et al., 2014). At the same time, the inventors also showed that K-H was responsible for the protein-protein stabilization of Artemis, a stabilization requiring an intact RPR domain, which competed for binding to the CTD domain in RNAPII (Morales et al., 2014). Depletion of K-H levels resulted in significant loss of steady state Artemis levels. As a result, the concomitant loss of RNA transcription termination and Artemis levels resulted in a phenotype whereby DSBs are created, but then cannot be repaired. As a result, elevated levels of complex DSBs, chromatid aberrations, hypersensitivity to DSB-inducing agents were noted (Morales et al., 2014; Morales et al., In Press and Morales et al., Submitted. 2015). K-H knockout mice were embryonic lethal and K-H KO cell lines did not grow, even in the absence of p53; K-H KO mice were not rescued by crossing with p53 KO mice (Morales et al., Submitted. 2015).

Higher-Order Protein Complexes Containing K-H or p15RS.

To better understand the separate functions of K-H, the inventors shifted to TAP-tag-gel filtration-Mass Spectrometry (MS) assays of complexes from nucleic acid-free nuclear or whole cell extracts (Morales et al., 2014; Patidar et al., 2015 and Patidar et al., 2014). In addition to RNA termination complexes, which were defined structurally and functionally (Morales et al., 2014 and Morales et al., Submitted. 2015), K-H formed various higher-order protein complexes that serve separate functions involved in stimulating specific gene transcription, or in Artemis-dependent NHEJ repair. A very complex higher-order protein elution was noted, containing both K-H and p15RS complexes, ±RNAPII. Complexes devoid of RNAPII were enriched for various DNA repair proteins, e.g., Ku and PARP1. The inventors then showed that the DSB repair complex contained accessory proteins that mediate Artemis-dependent repair of complex DSBs (Morales et al., 2014). This same complex also contained p15RS, which the invenros theorize is an accessory protein to Ku70 that: (i) forms foci at DSB sites; and (ii) facilitates classical NHEJ (cNHEJ). In delineating specific and separate K-H-containing higher-order protein complexes we noted those containing Artemis or RNAPII, but never both. This is consistent with the notion that RNAPII and Artemis compete for binding to the RPR domain of K-H to mediate repair or control transcription at CHR-containing genes, such as CDK1 (Motea et al., 2015).

K-H/p15RS Complexes in DSB Repair.

Further proteomic analyses of K-H's close homolog and protein binding partner, p15RS, revealed its association with K-H in both RNA termination and DSB repair complexes. Proteomic analyses strongly indicated numerous common binding proteins, e.g., DNA-PK and these were divided into those for transcription termination (RNAPII, Nono, PSF) or DSB repair (PARP1, DDB1, and DNA-PK). Unlike K-H, p15RS formed foci at DSBs.

K-H Structure Reveals Specific Functions—SNP Analyses of K-H: Molecular Modeled and Tumor-Derived Mutations, Including SNPs from Breast and NSCLC Cancers.

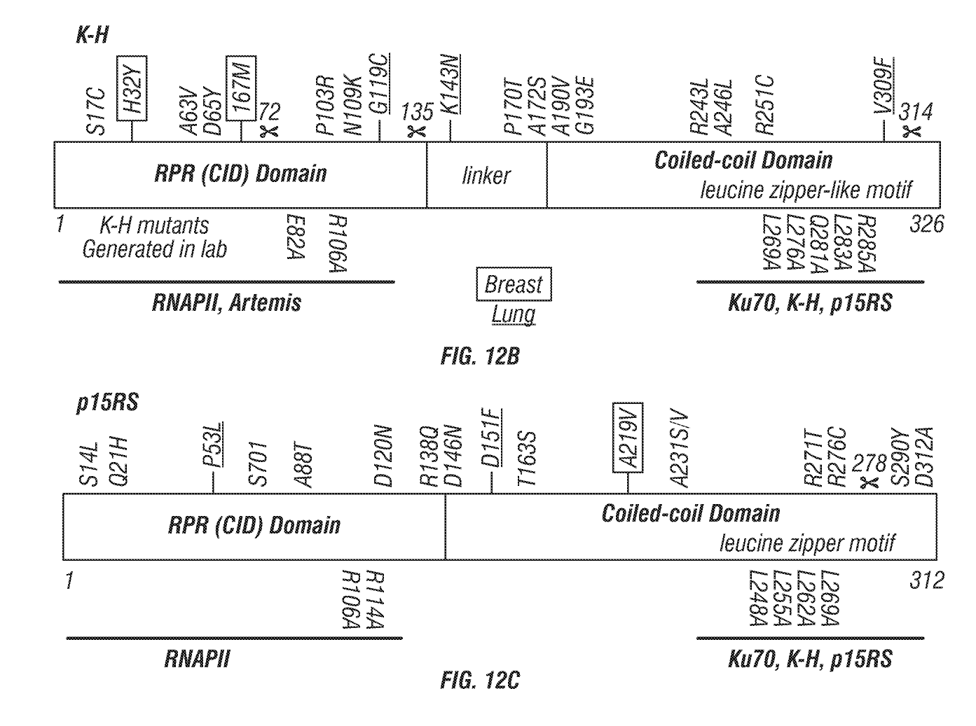

The molecular modeling of the proposed K-H-RNAPII interaction is shown in FIG. 12A. R106A was predicted to abrogate K-H's binding to RNAPII, and mutations found in tumor SNPs, including breast and NSCLC cancers selected to delineate effects of abrogating K-H or p15RS interactions with RNAPII are shown, Artemis, Ku70 and each other (heterodimers) or themselves (homodimers) (FIGS. 12B-C).

K-H is Required for Repair of Complex DSBs.

After cloning Ku70 binding protein #5 (Kub5) and sequencing the mammalian cDNA, we discovered that it was the homolog of the yeast RT103 gene. Analyses of RT103^{-/-}, yeast of shK-H-depleted mouse of human cells revealed that they were: (a) specifically defective at repair of complex DSBs (Morales et al., 2014); (b) Hypersensitive to DSB-inducing agents (IR, Topo I/II poisons, cisplatin, etc., but not UV), but only when cells were in a haploid state; and (c) Re-expression of yeast or human K-H cDNAs into haploid yeast corrected their hypersensitivities to DSB-inducing agents and capacity to repair complex DSBs (Morales et al., 2014).

Artemis Re-Expression Corrects Defective Complex DSB Repair, Restores Resistance, but not R-Loops, in K-H Depleted Cells.

Artemis is a unique DSB repair protein involved in the slow phase of repair typically lasting between 6-20 hr following IR (see below). shK-H-depleted human fibroblasts or 231 cells lacked Artemis expression, due to protein-protein stabilization of Artemis by K-H, while Artemis mRNA levels were unchanged (Morales et al., 2014). shK-H cells were hypersensitive to IR and other DSB-inducing agents, similar to Artemis-deficient cells. exhibited dramatic defects in DSB repair kinetics and were defective in NHEJ. Further analyses of shK-H cells showed that RNaseH1 or α-amanitin exposures corrected R-loop and basal DSB formation, but Art overexpression only corrected DNA repair, but not elevated persistent R-loops in K-H-depleted cells (Morales et al., 2014).

B. Artemis

Artemis is a protein that in humans is encoded by the DCLREIC (DNA gross-link repair 1C) gene. It is a nuclear protein that is involved in V(D)J recombination and DNA repair. The protein has endonuclease activity on 5′ and 3′ overhangs and hairpins when complexed with PRKDC. Mutations in Artemis cause Athabascan-type severe combined immunodeficiency (SCIDA), and Artemis has been shown to interact with DNA-PKcs.

Artemis plays an essential role in V(D)J recombination, the process by which B cell antibody genes and T cell receptor genes are assembled from individual V (variable), D (diversity), and J (joining) segments. For example, in joining a V segment to a D segment, the RAG (recombination activating gene) nuclease cuts both DNA strands adjacent to a V segment and adjacent to a D segment. The intervening DNA between the V and D segments is ligated to form a circular DNA molecule that is lost from the chromosome. At each of the two remaining ends, called the coding ends, the two strands of DNA are joined to form a hairpin structure. Artemis nuclease, in a complex with the DNA-dependent protein kinase (DNA-PK), binds to these DNA ends and makes a single cut near the tip of the hairpin. The exposed 3′ termini are subject to deletion and addition of nucleotides by a variety of exonucleases and DNA polymerases, before the V and D segments are ligated to restore the integrity of the chromosome. The exact site of cleavage of the hairpin by Artemis is variable, and this variability, combined with random nucleotide deletion and addition, confers extreme diversity upon the resulting antibody and T-cell receptor genes, thus allowing the immune system to mount an immune response to virtually any foreign antigen. In Artemis-deficient individuals, V(D)J recombination is blocked because the hairpin ends cannot be opened, and so no mature B or T cells are produced, a condition known as severe combined immune deficiency (SCID). Artemis was first identified as the gene defective in a subset of SCID patients that were unusually sensitive to radiation.

Cells deficient in Artemis are more sensitive than normal cells to X-rays and to chemical agents that induce double-strand breaks (DSBs), and they show a higher incidence of chromosome breaks following irradiation. Direct measurement of DSBs by pulsed-field electrophoresis indicates that in Artemis-deficient cells 75-90% of DSBs are repaired rapidly, just as in normal cells. However, the remaining 10-20% of DSBs that are repaired more slowly (2-24 hr) in normal cells, are not repaired at all in Artemis-deficient cells. Repair of these presumably difficult-to-rejoin breaks also requires several other proteins, including the Mre11/Rad50/NBS1 complex, the ataxia telangiectasia-mutated ATM kinase, and 53BP1. Because Artemis can remove damaged ends from DNA, it has been proposed that these DSBs are those whose damaged ends require trimming by Artemis. However, evidence that both ATM and Artemis are specifically required for repair of DSBs in heterochromatin, has called this interpretation into question.

C. CDK1

Cyclin-dependent kinase 1 (CDK1) or cell division cycle protein 2 homolog is a highly conserved protein that functions as a serine/threonine kinase, and is a key player in cell cycle regulation. It has been highly studied in the budding yeast S. cerevisiae, and the fission yeast S. pombe, where it is encoded by genes cdc28 and cdc2, respectively. In humans, Cdk1 is encoded by the CDC2 gene. With its cyclin partners, Cdk1 forms complexes that phosphorylate a variety of target substrates (over 75 have been identified in budding yeast); phosphorylation of these proteins leads to cell cycle progression.

Cdk1 is a small protein (approximately 34 kD), and is highly conserved. The human homolog of Cdk1, CDC2, shares approximately 63% amino-acid identity with its yeast homolog. Furthermore, human CDC2 is capable of rescuing fission yeast carrying a cdc2 mutation. Cdk1 is comprised mostly by the bare protein kinase motif, which other protein kinases share. Cdk1, like other kinases, contains a cleft in which ATP fits. Substrates of Cdk1 bind near the mouth of the cleft, and Cdk1 residues catalyze the covalent bonding of the γ-phosphate to the oxygen of the hydroxyl serine/threonine of the substrate. Cdk1 has been shown to interact with BCL2, CCNB1, CCNE1, CDKN3, DAB2, FANCC, GADD45A, LATS1, LYN, P53 and UBC.

In addition to this catalytic core, Cdk1, like other cyclin-dependent kinases, contains a T-loop, which, in the absence of an interacting cyclin, prevents substrate binding to the Cdk1 active site. Cdk1 also contains a PSTAIRE helix, which, upon cyclin binding, moves and rearranges the active site, facilitating Cdk1 kinase activities. When bound to its cyclin partners, Cdk1 phosphorylation leads to cell cycle progression. In the budding yeast, initial cell cycle entry is controlled by two regulatory complexes, SBF (SCB-binding factor) and MBF (MCB-binding factor). These two complexes control G₁/S gene transcription; however, they are normally inactive. SBF is inhibited by the protein Whi5; however, when phosphorylated by Cln3-Cdk1, Whi5 is ejected from the nucleus, allowing for transcription of the G₁/S regulon, which includes the G₁/S cyclins Cln1,2. G₁/S cyclin-Cdk1 activity leads to preparation for S phase entry (e.g., duplication of centromeres or the spindle pole body), and a rise in the S cyclins (Clb5,6 in S. cerevisiae). Clb5,6-Cdk1 complexes directly lead to replication origin initiation; however, they are inhibited by Sic1, preventing premature S phase initiation.

Cln1,2 and/or Clb5,6-Cdk1 complex activity leads to a sudden drop in Sic1 levels, allowing for coherent S phase entry. Finally, phosphorylation by M cyclins (e.g., Clb1, 2, 3 and 4) in complex with Cdk1 leads to spindle assembly and sister chromatid alignment. Cdk1 phosphorylation also leads to the activation of the ubiquitin-protein ligase APC^{Cdc20}, an activation which allows for chromatid segregation and, furthermore, degradation of M-phase cyclins. The destruction of M cyclins leads to the final events of mitosis (e.g., spindle disassembly, mitotic exit).

Given its essential role in cell cycle progression, Cdk1 is highly regulated. Most obviously, Cdk1 is regulated by its binding with its cyclin partners. Cyclin binding alters access to the active site of Cdk1, allowing for Cdk1 activity; furthermore, cyclins impart specificity to Cdk1 activity. At least some cyclins contain a hydrophobic patch which may directly interact with substrates, conferring target specificity. Furthermore, cyclins can target Cdk1 to particular subcellular locations.

In addition to regulation by cyclins, Cdk1 is regulated by phosphorylation. A conserved tyrosine (Tyr15 in humans) leads to inhibition of Cdk1; this phosphorylation is thought to alter ATP orientation, preventing efficient kinase activity. In S. pombe, for example, incomplete DNA synthesis may lead to stabilization of this phosphorylation, preventing mitotic progression. Weel, conserved among all eukaryotes phosphorylates Tyr15, whereas members of the Cdc25 family are phosphatases, counteracting this activity. The balance between the two is thought to help govern cell cycle progression. Weel is controlled upstream by Cdr1, Cdr2, and Pom1.

Cdk1-cyclin complexes are also governed by direct binding of Cdk inhibitor proteins (CKIs). One such protein, already discussed, is Sic1. Sic1 is a stoichiometric inhibitor that binds directly to Clb5,6-Cdk1 complexes. Multisite phosphorylation, by Cdk1-Cln1/2, of Sic1 is thought to time Sic1 ubiquitination and destruction, and by extension, the timing of S-phase entry. Only until Sic1 inhibition is overcome can Clb5,6 activity occur and S phase initiation may begin.

### II. CANCERS

A. Cancers

While hyper-proliferative disorders can be associated with any disease which causes a cell to begin to reproduce uncontrollably, the prototypical example is cancer. One of the key elements of cancer is that the cell's normal apoptotic cycle is interrupted and thus agents that interrupt the growth of the cells are important as therapeutic agents for treating these diseases. In this disclosure, the tubulysin analogs described herein may be used to lead to decreased cell counts and as such can potentially be used to treat a variety of types of cancer lines. In some aspects, it is anticipated that the tubulysin analogs described herein may be used to treat virtually any malignancy. Here, there is particular relevance to cancers over- or underexpressing Kub5/Hera, as well as cancers that contain mutations in significant functional domains of this protein.

Cancer cells derived from the bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus, may be treated according to the present disclosure. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

The following cancers have particular relevance to the present disclosure, as mutation and/or altered expression of Kub5/Hera has been demonstrated.

B. Breast Cancer

1. Background

Breast cancer is a neoplasm that starts in the breast, usually in the inner lining of the milk ducts or lobules. There are different types of breast cancer, with different stages (spread), aggressiveness, and genetic makeup. With best treatment, 10-year disease-free survival varies from 98% to 10%. Treatment is selected from surgery, drugs (chemotherapy), and radiation. In the United States, there were 216,000 cases of invasive breast cancer and 40,000 deaths in 2004. Worldwide, breast cancer is the second most common type of cancer after lung cancer (10.4% of all cancer incidence, both sexes counted) and the fifth most common cause of cancer death. In 2004, breast cancer caused 519,000 deaths worldwide (7% of cancer deaths; almost 1% of all deaths). Breast cancer is about 100 times as frequent among women as among men, but survival rates are equal in both sexes.

2. Symptoms

The first symptom, or subjective sign, of breast cancer is typically a lump that feels different from the surrounding breast tissue. According to the The Merck Manual, more than 80% of breast cancer cases are discovered when the woman feels a lump. According to the American Cancer Society, the first medical sign, or objective indication of breast cancer as detected by a physician, is discovered by mammogram. Lumps found in lymph nodes located in the armpits can also indicate breast cancer. Indications of breast cancer other than a lump may include changes in breast size or shape, skin dimpling, nipple inversion, or spontaneous single-nipple discharge. Pain (“mastodynia”) is an unreliable tool in determining the presence or absence of breast cancer, but may be indicative of other breast health issues.

When breast cancer cells invade the dermal lymphatics—small lymph vessels in the skin of the breast—its presentation can resemble skin inflammation and thus is known as inflammatory breast cancer (IBC). Symptoms of inflammatory breast cancer include pain, swelling, warmth and redness throughout the breast, as well as an orange-peel texture to the skin referred to as “peau d'orange.” Another reported symptom complex of breast cancer is Paget's disease of the breast. This syndrome presents as eczematoid skin changes such as redness and mild flaking of the nipple skin. As Paget's advances, symptoms may include tingling, itching, increased sensitivity, burning, and pain. There may also be discharge from the nipple. Approximately half of women diagnosed with Paget's also have a lump in the breast.

Occasionally, breast cancer presents as metastatic disease, that is, cancer that has spread beyond the original organ. Metastatic breast cancer will cause symptoms that depend on the location of metastasis. Common sites of metastasis include bone, liver, lung and brain. Unexplained weight loss can occasionally herald an occult breast cancer, as can symptoms of fevers or chills. Bone or joint pains can sometimes be manifestations of metastatic breast cancer, as can jaundice or neurological symptoms. These symptoms are “non-specific,” meaning they can also be manifestations of many other illnesses.

3. Risk Factors

The primary risk factors that have been identified are sex, age, childbearing, hormones, a high-fat diet, alcohol intake, obesity, and environmental factors such as tobacco use, radiation and shiftwork. No etiology is known for 95% of breast cancer cases, while approximately 5% of new breast cancers are attributable to hereditary syndromes. In particular, carriers of the breast cancer susceptibility genes, BRCA1 and BRCA2, are at a 30-40% increased risk for breast and ovarian cancer, depending on in which portion of the protein the mutation occurs. Experts believe that 95% of inherited breast cancer can be traced to one of these two genes. Hereditary breast cancers can take the form of a site-specific hereditary breast cancer—cancers affecting the breast only—or breast-ovarian and other cancer syndromes. Breast cancer can be inherited both from female and male relatives.

4. Subtypes

Breast cancer subtypes are typically categorized on an immunohistochemical basis. Subtype definitions are generally as follows:
normal (ER+, PR+, HER2+, cytokeratin 5/6+, and HER1+)
luminal A (ER+ and/or PR+, HER2−)
luminal B (ER+ and/or PR+, HER2+)
triple-negative (ER−, PR−, HER2−)
HER2+/ER−(ER−, PR−, and HER2+)
unclassified (ER−, PR−, HER2−, cytokeratin 5/6−, and HER1−)
   In the case of triple-negative breast cancer cells, the cancer's growth is not driven by estrogen or progesterone, or by growth signals coming from the HER2 protein. By the same token, such cancer cells do not respond to hormonal therapy, such as tamoxifen or aromatase inhibitors, or therapies that target HER2 receptors, such as Herceptin®. About 10-20% of breast cancers are found to be triple-negative. It is important to identify these types of cancer so that one can avoid costly and toxic effects of therapies that are unlike to succeed, and to focus on treatments that can be used to treat triple-negative breast cancer. Like other forms of breast cancer, triple-negative breast cancer can be treated with surgery, radiation therapy, and/or chemotherapy. One particularly promosing approach is “neoadjuvant” therapy, where chemo- and/or radiotherapy is provided prior to sugery. Another drug therapy is the use of poly (ADP-ribose) polymerase 1, or PARP1 inhibitors (PARP1i).

5. Traditional Screening and Diagnosis

While screening techniques discussed above are useful in determining the possibility of cancer, a further testing is necessary to confirm whether a lump detected on screening is to cancer, as opposed to a benign alternative such as a simple cyst. In a clinical setting, breast cancer is commonly diagnosed using a “triple test” of clinical breast examination (breast examination by a trained medical practitioner), mammography, and fine needle aspiration cytology. Both mammography and clinical breast exam, also used for screening, can indicate an approximate likelihood that a lump is cancer, and may also identify any other lesions. Fine Needle Aspiration and Cytology (FNAC), performed as an outpatient procedure using local anaesthetic, involves attempting to extract a small portion of fluid from the lump. Clear fluid makes the lump highly unlikely to be cancerous, but bloody fluid may be sent off for inspection under a microscope for cancerous cells. Together, these three tools can be used to diagnose breast cancer with a good degree of accuracy. Other options for biopsy include core biopsy, where a section of the breast lump is removed, and an excisional biopsy, where the entire lump is removed.

Breast cancer screening is an attempt to find cancer in otherwise healthy individuals. The most common screening method for women is a combination of x-ray mammography and clinical breast exam. In women at higher than normal risk, such as those with a strong family history of cancer, additional tools may include genetic testing or breast Magnetic Resonance Imaging (MRI).

Breast self-examination was a form of screening that was heavily advocated in the past, but has since fallen into disfavor since several large studies have shown that it does not have a survival benefit for women and often causes considerably anxiety. This is thought to be because cancers that could be detected tended to be at a relatively advanced stage already, whereas other methods push to identify the cancer at an earlier stage where curative treatment is more often possible.

X-ray mammography uses x-rays to examine the breast for any uncharacteristic masses or lumps. Regular mammograms are recommended in several countries in women over a certain age as a screening tool.

Genetic testing for breast cancer typically involves testing for mutations in the BRCA genes. This is not generally a recommended technique except for those at elevated risk for breast cancer.

6. Treatments

The mainstay of breast cancer treatment is surgery when the tumor is localized, with possible adjuvant hormonal therapy (with tamoxifen or an aromatase inhibitor), chemotherapy, and/or radiotherapy. At present, the treatment recommendations after surgery (adjuvant therapy) follow a pattern. Depending on clinical criteria (age, type of cancer, size, metastasis) patients are roughly divided into high risk and low risk cases, with each risk category following different rules for therapy. Treatment possibilities include radiation therapy, chemotherapy, hormone therapy, and immune therapy.

Targeted cancer therapies are treatments that target specific characteristics of cancer cells, such as a protein that allows the cancer cells to grow in a rapid or abnormal way. Targeted therapies are generally less likely than chemotherapy to harm normal, healthy cells. Some targeted therapies are antibodies that work like the antibodies made naturally by one's immune system. These types of targeted therapies are sometimes called immune-targeted therapies.

There are currently three targeted therapies doctors use to treat breast cancer. Herceptin® (trastuzumab) works against HER2-positive breast cancers by blocking the ability of the cancer cells to receive chemical signals that tell the cells to grow. Tykerb® (lapatinib) works against HER2-positive breast cancers by blocking certain proteins that can cause uncontrolled cell growth. Avastin® (bevacizumab) works by blocking the growth of new blood vessels that cancer cells depend on to grow and function.

Hormonal (anti-estrogen) therapy works against hormone-receptor-positive breast cancer in two ways: first, by lowering the amount of the hormone estrogen in the body, and second, by blocking the action of estrogen in the body. Most of the estrogen in women's bodies is made by the ovaries. Estrogen makes hormone-receptor-positive breast cancers grow. So reducing the amount of estrogen or blocking its action can help shrink hormone-receptor-positive breast cancers and reduce the risk of hormone-receptor-positive breast cancers coming back (recurring). Hormonal therapy medicines are not effective against hormone-receptor-negative breast cancers.

There are several types of hormonal therapy medicines, including aromatase inhibitors, selective estrogen receptor modulators, and estrogen receptor downregulators. In some cases, the ovaries and fallopian tubes may be surgically removed to treat hormone-receptor-positive breast cancer or as a preventive measure for women at very high risk of breast cancer. The ovaries also may be shut down temporarily using medication.

In planning treatment, doctors can also use PCR tests like Oncotype DX or microarray tests that predict breast cancer recurrence risk based on gene expression. In February 2007, the first breast cancer predictor test won formal approval from the Food and Drug Administration. This is a new gene test to help predict whether women with early-stage breast cancer will relapse in 5 or 10 years, this could help influence how aggressively the initial tumor is treated.

Radiation therapy is also used to help destroy cancer cells that may linger after surgery. Radiation can reduce the risk of recurrence by 50-66% when delivered in the correct dose.

C. Lung Cancer

1. Small Cell Lung Cancer

Small-cell carcinoma (also known as “small-cell lung cancer” or “oat-cell carcinoma”) is a type of highly malignant cancer that most commonly arises within the lung, although it can occasionally arise in other body sites, such as the cervix, prostate, and gastrointestinal tract. Compared to non-small cell carcinoma, small cell carcinoma has a shorter doubling time, higher growth fraction, and earlier development of metastases. 15% of lung cancers in the US are of this type. Small cell lung cancer occurs almost exclusively in smokers; most commonly in heavy smokers and rarely in non-smokers. Small-cell carcinoma of the lung usually presents in the central airways and infiltrates the submucosa leading to narrowing of bronchial airways. Common symptoms include cough, dyspnea, weight loss, and debility. Over 70% of patients with small-cell carcinoma presents with metastatic disease; common sites include liver, adrenals, bone, and brain.

Small-cell carcinoma is an undifferentiated neoplasm composed of primitive-appearing cells. As the name implies, the cells in small-cell carcinomas are smaller than normal cells, and barely have room for any cytoplasm. Some researchers identify this as a failure in the mechanism that controls the size of the cells.

Due to its high grade neuroendocrine nature, small-cell carcinomas can produce ectopic hormones, including adrenocorticotropic hormone (ACTH) and anti-diuretic hormone (ADH). Ectopic production of large amounts of ADH leads to syndrome of inappropriate antidiuretic hormone hypersecretion (SIADH). Lambert-Eaton myasthenic syndrome (LEMS) is a well-known paraneoplastic condition linked to small-cell carcinoma.

When associated with the lung, it is sometimes called “oat cell carcinoma” due to the flat cell shape and scanty cytoplasm.

It is thought to originate from neuroendocrine cells (APUD cells) in the bronchus called Feyrter cells. Hence, they express a variety of neuroendocrine markers, and may lead to ectopic production of hormones like ADH and ACTH that may result in paraneoplastic syndromes and Cushing's syndrome. Approximately half of all individuals diagnosed with Lambert-Eaton myasthenic syndrome (LEMS) will eventually be found to have a small-cell carcinoma of the lung.

Small-cell carcinoma is most often more rapidly and widely metastatic than non-small cell lung carcinoma (and hence staged differently). There is usually early involvement of the hilar and mediastinal lymph nodes.

Small-cell lung carcinoma can occur in combination with a wide variety of other histological variants of lung cancer, including extremely complex malignant tissue admixtures. When it is found with one or more differentiated forms of lung cancer, such as squamous cell carcinoma or adenocarcinoma, the malignant tumor is then diagnosed and classified as a combined small cell lung carcinoma (c-SCLC). C-SCLC is the only currently recognized subtype of SCLC.

Although combined small-cell lung carcinoma is currently staged and treated similarly to “pure” small-cell carcinoma of the lung, recent research suggests surgery might improve outcomes in very early stages of this tumor type. Smoking is a significant etiological factor.

Symptoms and signs are as for other lung cancers. In addition, because of their neuroendocrine cell origin, small-cell carcinomas will often secrete substances that result in paraneoplastic syndromes such as Lambert-Eaton myasthenic syndrome.

Very rarely, the primary site for small-cell carcinoma is outside of the lungs and pleural space, it is referred to as extrapulmonary small-cell carcinoma (EPSCC). Outside of the respiratory tract, small-cell carcinoma can appear in the cervix, prostate, liver, pancreas, gastrointestinal tract, or bladder. It is estimated to account for 1,000 new cases a year in the U.S. Histologically similar to small-cell lung cancer, therapies for small-cell lung cancer are usually used to treat EPSCC. First line treatment is usually with cisplatin and etoposide. In Japan, the first line treatment is shifting to irinotecan and cisplatin.

When the primary site is in the skin, it is referred to as Merkel cell carcinoma. Extrapulmonary small-cell carcinoma localized in the lymph nodes. This is an extremely rare type of small cell, and there has been little information in the scientific community. It appears to occur in only one or more lymph nodes, and nowhere else in the body. Treatment is similar to small cell lung cancer, but survival rates are much higher than other small-cell carcinomas.

In the prostate, small-cell carcinoma (SCCP) is a rare form of cancer (approx 1% of PC). Due to the fact that there is little variation in prostate specific antigen levels, this form of cancer is normally diagnosed at an advanced stage, after metastasis. It can metastasize to the brain.

Small-cell lung carcinoma has long been divided into two clinicopathological stages, including limited stage (LS) and extensive stage (ES). The stage is generally determined by the presence or absence of metastases, whether or not the tumor appears limited to the thorax, and whether or not the entire tumor burden within the chest can feasibly be encompassed within a single radiotherapy portal. In general, if the tumor is confined to one lung and the lymph nodes close to that lung, the cancer is said to be LS. If the cancer has spread beyond that, it is said to be ES.

In cases of LS-SCLC, combination chemotherapy (often including cyclophosphamide, cisplatinum, doxorubicin, etoposide, vincristine and/or paclitaxel) is administered together with concurrent chest radiotherapy (RT). Chest RT has been shown to improve survival in LS-SCLC. Exceptionally high objective initial response rates (RR) of between 60% and 90% are seen in LS-SCLC using chemotherapy alone, with between 45% and 75% of individuals showing a “complete response” (CR), which is defined as the disappearance of all radiological and clinical signs of tumor. Unfortunately, relapse is the rule, and median survival is only 18 to 24 months.

Because SCLC usually metastasizes widely very early on in the natural history of the tumor, and because nearly all cases respond dramatically to CT and/or RT, there has been little role for surgery in this disease since the 1970s. However, recent work suggests that in cases of small, asymptomatic, node-negative SCLC's (“very limited stage”), surgical excision may improve survival when used prior to chemotherapy. (“adjuvant chemotherapy”).

In ES-SCLC, combination chemotherapy is the standard of care, with radiotherapy added only to palliate symptoms such as dyspnea, pain from liver or bone metastases, or for treatment of brain metastases, which, in small-cell lung carcinoma, typically have a rapid, if temporary, response to whole brain radiotherapy.

Combination chemotherapy consists of a wide variety of agents, including cisplatin, cyclophosphamide, vincristine and carboplatin. Response rates are high even in extensive disease, with between 15% and 30% of subjects having a complete response to combination chemotherapy, and the vast majority having at least some objective response. Responses in ES-SCLC are often of short duration, however.

If complete response to chemotherapy occurs in a subject with SCLC, then prophylactic cranial irradiation (PCI) is often used in an attempt to prevent the emergence of brain metastases. Although this treatment is often effective, it can cause hair loss and fatigue. Prospective randomized trials with almost two years follow-up have not shown neurocognitive ill-effects. Meta-analyses of randomized trials confirm that PCI provides significant survival benefits.

All in all, small-cell carcinoma is very responsive to chemotherapy and radiotherapy, and in particular, regimens based on platinum-containing agents. However, most people with the disease relapse, and median survival remains low.

In limited-stage disease, median survival with treatment is 14-20 months, and about 20% of patients with limited-stage small-cell lung carcinoma live 5 years or longer. Because of its predisposition for early metastasis, the prognosis of SCLC is poor, with only 10% to 15% of patients surviving 3 years. The prognosis is far more grim in extensive-stage small-cell lung carcinoma; with treatment, median survival is 8-13 months; only 1-5% of patients with extensive-stage small-cell lung carcinoma treated with chemotherapy live 5 years or longer.

2. Non-Small Cell Lung Cancer

Non-small-cell lung carcinoma (NSCLC) is any type of epithelial lung cancer other than small cell lung carcinoma (SCLC). As a class, NSCLCs are relatively insensitive to chemotherapy, compared to small cell carcinoma. When possible, they are primarily treated by surgical resection with curative intent, although chemotherapy is increasingly being used both pre-operatively (neoadjuvant chemotherapy) and post-operatively (adjuvant chemotherapy).

The most common types of NSCLC are squamous cell carcinoma, large cell carcinoma, and adenocarcinoma, but there are several other types that occur less frequently, and all types can occur in unusual histologic variants and as mixed cell-type combinations. Sometimes the phrase “non-small-cell lung cancer” (“not otherwise specified”, or NOS) is used generically, usually when a more specific diagnosis cannot be made. This is most often the case when a pathologist examines a small amount of malignant cells or tissue in a cytology or biopsy specimen.

Lung cancer in never-smokers is almost universally NSCLC, with a sizeable majority being adenocarcinoma. On relatively rare occasions, malignant lung tumors are found to contain components of both SCLC and NSCLC. In these cases, the tumors should be classified as combined small cell lung carcinoma (c-SCLC), and are (usually) treated like “pure” SCLC.

Adenocarcinoma of the lung is currently the most common type of lung cancer in “never smokers” (lifelong non-smokers). Adenocarcinomas account for approximately 40% of lung cancers. Historically, adenocarcinoma was more often seen peripherally in the lungs than small cell lung cancer and squamous cell lung cancer, both of which tended to be more often centrally located. Interestingly, however, recent studies suggest that the “ratio of centrally-to-peripherally occurring” lesions may be converging toward unity for both adenocarcinoma and squamous cell carcinoma.

Squamous cell carcinoma (SCC) of the lung is more common in men than in women. It is closely correlated with a history of tobacco smoking, more so than most other types of lung cancer. According to the Nurses' Health Study, the relative risk of SCC is approximately 5.5, both among those with a previous duration of smoking of 1 to 20 years, and those with to 30 years, compared to never-smokers. The relative risk increases to approximately 16 with a previous smoking duration of 30 to 40 years, and approximately 22 with more than 40 years.

Large cell lung carcinoma (LCLC) is a heterogeneous group of undifferentiated malignant neoplasms originating from transformed epithelial cells in the lung. LCLC's have typically comprised around 10% of all NSCLC in the past, although newer diagnostic techniques seem to be reducing the incidence of diagnosis of “classic” LCLC in favor of more poorly differentiated squamous cell carcinomas and adenocarcinomas. LCLC is, in effect, a “diagnosis of exclusion”, in that the tumor cells lack light microscopic characteristics that would classify the neoplasm as a small-cell carcinoma, squamous-cell carcinoma, adenocarcinoma, or other more specific histologic type of lung cancer. LCLC is differentiated from small cell lung carcinoma (SCLC) primarily by the larger size of the anaplastic cells, a higher cytoplasmic-to-nuclear size ratio, and a lack of “salt-and-pepper” chromatin.

More than one kind of treatment is often used, depending on the stage of the cancer, the individual's overall health, age, response to chemotherapy, and other factors such as the likely side effects of the treatment. NSCLCs are usually not very sensitive to chemotherapy and/or radiation, so surgery is the treatment of choice if diagnosed at an early stage, often with adjuvant (ancillary) chemotherapy involving cisplatin. Other treatment choices are chemotherapy, radiation therapy (radiotherapy), and targeted therapy.

New methods of giving radiation treatment allow doctors to be more accurate in treating lung cancers. This means less radiation affects nearby healthy tissues. New methods include Cyberknife and stereotactic radiosurgery (SRS). Other treatments are radiofrequency ablationand chemoembolization.

A wide variety of chemotherapies are used in advanced (metastatic) NSCLC. Some patients with particular mutations in the EGFR gene respond to EGFR tyrosine kinase inhibitors such as gefitinib. About 7% of NSCLC have EML4-ALK translocations; these may benefit from ALK inhibitors which are in clinical trials. Crizotinib gained FDA approval in August 2011.

D. Lymphoma

Lymphomas are a group of blood cell tumors that develop from lymphatic tissues. The name often refers to just the cancerous ones rather than all such tumors. Symptoms may include enlarged lymph nodes that are not generally painful, fevers, sweats, itchiness, weight loss, and feeling tired, among others. The sweats are most common at night. Because the whole system is part of the body's immune system, patients with a weakened immune system such as from HIV infection or from certain drugs or medication also have a higher incidence of lymphoma.

Lymphoma is the most common form of hematological malignancy, or “blood cancer”, in the developed world. Taken together, lymphomas represent 5.3% of all cancers (excluding simple basal cell and squamous cell skin cancers) in the United States and 55.6% of all blood cancers. According to the U.S. National Institutes of Health, lymphomas account for about 5%, and Hodgkin lymphoma in particular accounts for less than 1% of all cases of cancer in the United States.

The two main types are Hodgkin lymphoma (HL) and non-Hodgkin lymphoma (NHL), with two others, multiple myeloma and immunoproliferative diseases, also included by the World Health Organization within the category. Non-Hodgkin lymphoma makes up about 90% of cases and includes a large number of subtypes. Lymphomas and leukemias are part of the broader group of tumors called tumors of the hematopoietic and lymphoid tissues.

Risk factors for HL include: infection with Epstein-Barr virus and having others in the family with the disease. Risk factors for NHL include: autoimmune diseases, HIV/AIDS, infection with human T-lymphotropic virus, eating a large amount of meat and fat, immunosuppressant medications, and some pesticides. They are usually diagnosed by blood, urine, or bone marrow testing. A biopsy of a lymph node may also be useful. Medical imaging then may be done to determine if and where the cancer has spread. This spread can occur to many other organs, including: lungs, liver, and brain.

Treatment may involve some combination of chemotherapy, radiation therapy, targeted therapy, and surgery. In NHL, the blood may become so thick with protein that a procedure called plasmapheresis is needed. Watchful waiting may be appropriate for certain types. Some types are curable. The overall five-year survival rate in the United States for HL is 85%, while that for NHL is 69%. Worldwide, lymphomas developed in 566,000 people in 2012 and caused 305,000 deaths. They make up 3-4% of all cancers, making them as a group the seventh-most common form. In children they are the third most common cancer. They occur more often in the developed world than the developing world.

Lymphoma presents with certain nonspecific symptoms. If symptoms are persistent, lymphoma needs to be excluded medically. Lymphadenopathy or swelling of lymph nodes is the primary presentation in lymphoma. B symptoms (systemic symptoms) can be associated with both Hodgkin lymphoma and non-Hodgkin lymphoma. They consist of fever, night sweats, and weight loss, Other symptoms include loss of appetite or anorexia, fatigue, respiratory distress or dyspnea and itching.

Lymphomas sensu stricto are neoplasms of the lymphatic tissues. The main class are malignant neoplasms (that is, cancer) of the lymphocytes, a type of white blood cell that belongs to both the lymph and the blood and pervades both. Thus lymphomas and leukemias are both tumors of the hematopoietic and lymphoid tissues, and as lymphoproliferative disorders, lymphomas and lymphoid leukemias are closely related, to the point that some of them are unitary disease entities that can be called by either name (for example, adult T-cell leukemia/lymphoma).

Lymphoma is definitively diagnosed by a lymph node biopsy, meaning a partial or total excision of a lymph node examined under the microscope. This examination reveals histopathological features that may indicate lymphoma. After lymphoma is diagnosed, a variety of tests may be carried out to look for specific features characteristic of different types of lymphoma. These include immunophenotyping, flow cytometry and fluorescence in situ hybridization testing.

Several classification systems have existed for lymphoma, which use histological and other findings to divide lymphoma into different categories. The classification of lymphoma can affect treatment and prognosis. Classification systems generally classify lymphoma according to:
whether or not it is a Hodgkin lymphoma
whether the cell that is replicating is a T cell or B cell
the site from which the cell arises

1. Hodgkin Lymphoma

Hodgkin lymphoma is one of the most commonly known types of lymphoma, and differs from other forms of lymphoma in its prognosis and several pathological characteristics. A division into Hodgkin and non-Hodgkin lymphomas is used in several of the older classification systems. A Hodgkin lymphoma is marked by the presence of a type of cell called the Reed-Steinberg cell.

2. Non-Hodgkin Lymphomas

Non-Hodgkin lymphomas, which are defined as being all lymphomas except Hodgkin lymphoma, are more common than Hodgkin lymphoma. There is a very wide variety of lymphomas in this class, and the causes, the types of cells involved, and the prognosis varies by type. The incidence of non-Hodgkin lymphoma increases with age.

3. Staging

After a diagnosis and before treatment, a cancer is staged. This refers to deducing how far the cancer has spread, in local tissue and to other sites. Staging is reported as a grade between I (confined) and IV (spread). Staging is carried out because the stage of a cancer impacts its prognosis and treatment.

The Ann Arbor staging system is routinely used for staging of both HL and NHL. In this staging system, I represents a localized disease contained within a lymph node, II represents the presence of lymphoma in two or more lymph nodes, III represents spread of the lymphoma to both sides of the diaphragm, and IV indicates tissue outside a lymph node.

CT scan or PET scan imaging modalities are used to stage a cancer. Age and poor performance status are established poor prognostic factors, as well.

4. Prognosis and Treatment

Prognoses and treatments are different for HL and between all the different forms of NHL, and also depend on the grade of tumor, referring to how quickly a cancer replicates. Paradoxically, high-grade lymphomas are more readily treated and have better prognoses. A well-known example of a high-grade tumor is that of Burkitt lymphoma, which is a high-grade tumor known to double within days, but is readily treated. Lymphomas may be curable if detected in early stages with modern treatment.

Many low-grade lymphomas remain indolent for many years. Treatment of the non-symptomatic patient is often avoided. In these forms of lymphoma, such as follicular lymphoma, watchful waiting is often the initial course of action. This is carried out because the harms and risks of treatment outweigh the benefits. If a low-grade lymphoma is becoming symptomatic, radiotherapy or chemotherapy are the treatments of choice; although they do not cure the lymphoma, they can alleviate the symptoms, particularly painful lymphadenopathy. Patients with these types of lymphoma can live near-normal lifespans, but the disease is incurable. Some centers advocate the use of single agent rituximab in the treatment of follicular lymphoma rather than the wait and watch approach. Watchful waiting is not a good strategy for all patients, as it leads to significant distress and anxiety in some patients. It has been equated with watch and worry.

Treatment of some other, more aggressive, forms of lymphoma, often referred to as “high grade,” can result in a cure in the majority of cases, but the prognosis for patients with a poor response to therapy is worse. Treatment for these types of lymphoma typically consists of aggressive chemotherapy, including the CHOP or R-CHOP regimen. A number of people are cured with first-line chemotherapy. Most patients relapse within the first two years, and the relapse risk drops significantly thereafter. For people who relapse, high-dose chemotherapy followed by autologous stem cell transplantation is a proven approach.

Hodgkin lymphoma typically is treated with radiotherapy alone, as long as it is localized. Advanced Hodgkin disease requires systemic chemotherapy, sometimes combined with radiotherapy. Chemotherapy used includes the ABVD regimen, which is commonly used in the United States. Other regimens used in the management of Hodgkin lymphoma include BEACOPP and Stanford V. Considerable controversy exists regarding the use of ABVD or BEACOPP. Briefly, both regimens are effective, but BEACOPP is associated with more toxicity. Encouragingly, a significant number of people who relapse after ABVD can still be salvaged by stem cell transplant.

E. Ovarian Cancer

Ovarian cancer is a cancerous growth arising from different parts of the ovary. Most (>90%) ovarian cancers are classified as “epithelial” and were believed to arise from the surface (epithelium) of the ovary. However, recent evidence suggests that the Fallopian tube could also be the source of some ovarian cancers. Since the ovaries and tubes are closely related to each other, it is hypothesized that these cells can mimic ovarian cancer. Other types arise from the egg cells (germ cell tumor) or supporting cells (sex cord/stromal).

In 2004, in the United States, 25,580 new cases were diagnosed and 16,090 women died of ovarian cancer. The risk increases with age and decreases with pregnancy. Lifetime risk is about 1.6%, but women with affected first-degree relatives have a 5% risk. Women with a mutated BRCA1 or BRCA2 gene carry a risk between 25% and 60% depending on the specific mutation. Ovarian cancer is the fifth leading cause of death from cancer in women and the leading cause of death from gynecological cancer.

Ovarian cancer causes non-specific symptoms. Early diagnosis would result in better survival, on the assumption that stage I and II cancers progress to stage III and IV cancers (but this has not been proven). Most women with ovarian cancer report one or more symptoms such as abdominal pain or discomfort, an abdominal mass, bloating, back pain, urinary urgency, constipation, tiredness and a range of other non-specific symptoms, as well as more specific symptoms such as pelvic pain, abnormal vaginal bleeding or involuntary weight loss. There can be a build-up of fluid (ascites) in the abdominal cavity.

Diagnosis of ovarian cancer starts with a physical examination (including a pelvic examination), a blood test (for CA-125 and sometimes other markers), and transvaginal ultrasound. The diagnosis must be confirmed with surgery to inspect the abdominal cavity, take biopsies (tissue samples for microscopic analysis) and look for cancer cells in the abdominal fluid. Treatment usually involves chemotherapy and surgery, and sometimes radiotherapy.

In most cases, the cause of ovarian cancer remains unknown. Older women, and in those who have a first or second degree relative with the disease, have an increased risk. Hereditary forms of ovarian cancer can be caused by mutations in specific genes (most notably BRCA1 and BRCA2, but also in genes for hereditary non-polyposis colorectal cancer, such as MLH1 and PMS2). Infertile women and those with a condition called endometriosis, those who have never been pregnant and those who use postmenopausal estrogen replacement therapy are at increased risk. Use of combined oral contraceptive pills is a protective factor. The risk is also lower in women who have had their uterine tubes blocked surgically (tubal ligation).

Ovarian cancer is classified according to the histology of the tumor, obtained in a pathology report. Histology dictates many aspects of clinical treatment, management, and prognosis. Surface epithelial-stromal tumor, also known as ovarian epithelial carcinoma, is the most common type of ovarian cancer. It includes serous tumor, endometrioid tumor and mucinous cystadenocarcinoma. Sex cord-stromal tumor, including estrogen-producing granulosa cell tumor and virilizing Sertoli-Leydig cell tumor or arrhenoblastoma, accounts for 8% of ovarian cancers. Germ cell tumor accounts for approximately 30% of ovarian tumors but only 5% of ovarian cancers, because most germ cell tumors are teratomas and most teratomas are benign (see Teratoma). Germ cell tumor tends to occur in young women and girls. The prognosis depends on the specific histology of germ cell tumor, but overall is favorable. Mixed tumors, containing elements of more than one of the above classes of tumor histology.

Ovarian cancer can also be a secondary cancer, the result of metastasis from a primary cancer elsewhere in the body. Seven percent of ovarian cancers are due to metastases while the rest are primary cancers. Common primary cancers are breast cancer and gastrointestinal cancer (a common mistake is to name all peritoneal metastases from any gastrointestinal cancer as Krukenberg cancer, but this is only the case if it originates from primary gastric cancer). Surface epithelial-stromal tumor can originate in the peritoneum (the lining of the abdominal cavity), in which case the ovarian cancer is secondary to primary peritoneal cancer, but treatment is basically the same as for primary surface epithelial-stromal tumor involving the peritoneum.

Ovarian cancer staging is by the FIGO staging system and uses information obtained after surgery, which can include a total abdominal hysterectomy, removal of (usually) both ovaries and fallopian tubes, (usually) the omentum, and pelvic (peritoneal) washings for cytopathology. The AJCC stage is the same as the FIGO stage. The AJCC staging system describes the extent of the primary Tumor (T), the absence or presence of metastasis to nearby lymph Nodes (N), and the absence or presence of distant Metastasis (M).

The AJCC/TNM staging system includes three categories for ovarian cancer, T, N and M. The T category contains three other subcategories, T1, T2 and T3, each of them being classified according to the place where the tumor has developed (in one or both ovaries, inside or outside the ovary). The T1 category of ovarian cancer describes ovarian tumors that are confined to the ovaries, and which may affect one or both of them. The sub-subcategory T1a is used to stage cancer that is found in only one ovary, which has left the capsule intact and which cannot be found in the fluid taken from the pelvis. Cancer that has not affected the capsule, is confined to the inside of the ovaries and cannot be found in the fluid taken from the pelvis but has affected both ovaries is staged as T1b. T1c category describes a type of tumor that can affect one or both ovaries, and which has grown through the capsule of an ovary or it is present in the fluid taken from the pelvis. T2 is a more advanced stage of cancer. In this case, the tumor has grown in one or both ovaries and is spread to the uterus, fallopian tubes or other pelvic tissues. Stage T2a is used to describe a cancerous tumor that has spread the uterus or the fallopian tubes (or both) but which is not present in the fluid taken from the pelvis. Stages T2b and T2c indicate cancer that metastasized to other pelvic tissues than the uterus and fallopian tubes and which cannot be seen in the fluid taken from the pelvis, respectively tumors that spread to any of the pelvic tissues (including uterus and fallopian tubes) but which can also be found in the fluid taken from the pelvis. T3 is the stage used to describe cancer that has spread to the peritoneum. This stage provides information on the size of the metastatic tumors (tumors that are located in other areas of the body, but are caused by ovarian cancer). These tumors can be very small, visible only under the microscope (T3a), visible but not larger than 2 centimeters (T3b) and bigger than 2 centimeters (T3c).

This staging system also uses N categories to describe cancers that have or not spread to nearby lymph nodes. There are only two N categories, N0 which indicates that the cancerous tumors have not affected the lymph nodes, and N1 which indicates the involvement of lymph nodes close to the tumor. The M categories in the AJCC/TNM staging system provide information on whether the ovarian cancer has metastasized to distant organs such as liver or lungs. M0 indicates that the cancer did not spread to distant organs and M1 category is used for cancer that has spread to other organs of the body. The AJCC/TNM staging system also contains a Tx and a Nx sub-category which indicates that the extent of the tumor cannot be described because of insufficient data, respectively the involvement of the lymph nodes cannot be described because of the same reason.

Ovarian cancer, as well as any other type of cancer, is also graded, apart from staged. The histologic grade of a tumor measures how abnormal or malignant its cells look under the microscope. There are four grades indicating the likelihood of the cancer to spread and the higher the grade, the more likely for this to occur. Grade 0 is used to describe non-invasive tumors. Grade 0 cancers are also referred to as borderline tumors. Grade 1 tumors have cells that are well differentiated (look very similar to the normal tissue) and are the ones with the best prognosis. Grade 2 tumors are also called moderately well differentiated and they are made up by cells that resemble the normal tissue. Grade 3 tumors have the worst prognosis and their cells are abnormal, referred to as poorly differentiated.

The signs and symptoms of ovarian cancer are most of the times absent, but when they exist they are nonspecific. In most cases, the symptoms persist for several months until the patient is diagnosed. A prospective case-control study of 1,709 women visiting primary care clinics found that the combination of bloating, increased abdominal size, and urinary symptoms was found in 43% of those with ovarian cancer but in only 8% of those presenting to primary care clinics.

The exact cause is usually unknown. The risk of developing ovarian cancer appears to be affected by several factors. The more children a woman has, the lower her risk of ovarian cancer. Early age at first pregnancy, older age of final pregnancy and the use of low dose hormonal contraception have also been shown to have a protective effect. Ovarian cancer is reduced in women after tubal ligation.

The relationship between use of oral contraceptives and ovarian cancer was shown in a summary of results of 45 case-control and prospective studies. Cumulatively these studies show a protective effect for ovarian cancers. Women who used oral contraceptives for 10 years had about a 60% reduction in risk of ovarian cancer. (risk ratio 0.42 with statistical significant confidence intervals given the large study size, not unexpected). This means that if 250 women took oral contraceptives for 10 years, 1 ovarian cancer would be prevented. This is by far the largest epidemiological study to date on this subject (45 studies, over 20,000 women with ovarian cancer and about 80,000 controls).

There is good evidence that in some women genetic factors are important. Carriers of certain mutations of the BRCA1 or the BRCA2 gene are notably at risk. The BRCA1 and BRCA2 genes account for 5%-13% of ovarian cancers and certain populations (e.g., Ashkenazi Jewish women) are at a higher risk of both breast cancer and ovarian cancer, often at an earlier age than the general population. Patients with a personal history of breast cancer or a family history of breast and/or ovarian cancer, especially if diagnosed at a young age, may have an elevated risk.

A strong family history of uterine cancer, colon cancer, or other gastrointestinal cancers may indicate the presence of a syndrome known as hereditary nonpolyposis colorectal cancer (HNPCC, also known as Lynch syndrome), which confers a higher risk for developing ovarian cancer. Patients with strong genetic risk for ovarian cancer may consider the use of prophylactic, i.e., preventative, oophorectomy after completion of childbearing. Australia being member of International Cancer Genome Consortium is leading efforts to map ovarian cancer's complete genome.

Ovarian cancer at its early stages (I/II) is difficult to diagnose until it spreads and advances to later stages (III/IV). This is because most symptoms are non-specific and thus of little use in diagnosis. When an ovarian malignancy is included in the list of diagnostic possibilities, a limited number of laboratory tests are indicated. A complete blood count (CBC) and serum electrolyte test should be obtained in all patients. The serum BHCG level should be measured in any female in whom pregnancy is a possibility. In addition, serum alpha-fetoprotein (AFP) and lactate dehydrogenase (LDH) should be measured in young girls and adolescents with suspected ovarian tumors because the younger the patient, the greater the likelihood of a malignant germ cell tumor. A blood test called CA-125 is useful in differential diagnosis and in follow up of the disease, but it by itself has not been shown to be an effective method to screen for early-stage ovarian cancer due to its unacceptable low sensitivity and specificity. However, this is the only widely-used marker currently available.

Current research is looking at ways to combine tumor markers proteomics along with other indicators of disease (i.e., radiology and/or symptoms) to improve accuracy. The challenge in such an approach is that the very low population prevalence of ovarian cancer means that even testing with very high sensitivity and specificity will still lead to a number of false positive results (i.e., performing surgical procedures in which cancer is not found intra-operatively). However, the contributions of proteomics are still in the early stages and require further refining. Current studies on proteomics mark the beginning of a paradigm shift towards individually tailored therapy.

A pelvic examination and imaging including CT scan and trans-vaginal ultrasound are essential. Physical examination may reveal increased abdominal girth and/or ascites (fluid within the abdominal cavity). Pelvic examination may reveal an ovarian or abdominal mass. The pelvic examination can include a rectovaginal component for better palpation of the ovaries. For very young patients, magnetic resonance imaging may be preferred to rectal and vaginal examination.

To definitively diagnose ovarian cancer, a surgical procedure to take a look into the abdomen is required. This can be an open procedure (laparotomy, incision through the abdominal wall) or keyhole surgery (laparoscopy). During this procedure, suspicious areas will be removed and sent for microscopic analysis. Fluid from the abdominal cavity can also be analyzed for cancerous cells. If there is cancer, this procedure can also determine its spread (which is a form of tumor staging).

Women who have had children are less likely to develop ovarian cancer than women who have not, and breastfeeding may also reduce the risk of certain types of ovarian cancer. Tubal ligation and hysterectomy reduce the risk and removal of both tubes and ovaries (bilateral salpingo-oophorectomy) dramatically reduces the risk of not only ovarian cancer but breast cancer also. The use of oral contraceptives (birth control pills) for five years or more decreases the risk of ovarian cancer in later life by 50%.

Tubal ligation is believed to decrease the chance of developing ovarian cancer by up to 67% while a hysterectomy may reduce the risk of getting ovarian cancer by about one-third. Moreover, according to some studies, analgesics such as acetaminophen and aspirin seem to reduce one's risks of developing ovarian cancer. Yet, the information is not consistent and more research needs to be performed.

Routine screening of women for ovarian cancer is not recommended by any professional society—this includes the U.S. Preventive Services Task Force, the American Cancer Society, the American College of Obstetricians and Gynecologists, and the National Comprehensive Cancer Network. This is because no trial has shown improved survival for women undergoing screening. Screening for any type of cancer must be accurate and reliable—it needs to accurately detect the disease and it must not give false positive results in people who do not have cancer. As yet there is no technique for ovarian screening that has been shown to fulfill these criteria. However, in some countries such as the UK, women who are likely to have an increased risk of ovarian cancer (for example, if they have a family history of the disease) can be offered individual screening through their doctors, although this will not necessarily detect the disease at an early stage.

Researchers are assessing different ways to screen for ovarian cancer. Screening tests that could potentially be used alone or in combination for routine screening include the CA-125 marker and transvaginal ultrasound. Doctors can measure the levels of the CA-125 protein in a woman's blood—high levels could be a sign of ovarian cancer, but this is not always the case. And not all women with ovarian cancer have high CA-125 levels. Transvaginal ultrasound involves using an ultrasound probe to scan the ovaries from inside the vagina, giving a clearer image than scanning the abdomen. The UK Collaborative Trial of Ovarian Cancer Screening is testing a screening technique that combines CA-125 blood tests with transvaginal ultrasound.

The purpose of screening is to diagnose ovarian cancer at an early stage, when it is more likely to be treated successfully. However, the development of the disease is not fully understood, and it has been argued that early-stage cancers may not always develop into late-stage disease. With any screening technique there are risks and benefits that need to be carefully considered, and health authorities need to assess these before introducing any ovarian cancer screening programs.

The goal of ovarian cancer screening is to detect the disease at stage I. Several large studies are ongoing, but none have identified an effective technique. In 2009, however, early results from the UK Collaborative Trial of Ovarian Cancer Screening (UKCTOCS) showed that a technique combining annual CA-125 tests with ultrasound imaging did help to detect the disease at an early stage. However, it is not yet clear if this approach could actually help to save lives—the full results of the trial will be published in 2015.

Surgical treatment may be sufficient for malignant tumors that are well-differentiated and confined to the ovary. Addition of chemotherapy may be required for more aggressive tumors that are confined to the ovary. For patients with advanced disease a combination of surgical reduction with a combination chemotherapy regimen is standard. Borderline tumors, even following spread outside of the ovary, are managed well with surgery, and chemotherapy is not seen as useful.

Surgery is the preferred treatment and is frequently necessary to obtain a tissue specimen for differential diagnosis via its histology. Surgery performed by a specialist in gynecologic oncology usually results in an improved result. Improved survival is attributed to more accurate staging of the disease and a higher rate of aggressive surgical excision of tumor in the abdomen by gynecologic oncologists as opposed to general gynecologists and general surgeons.

The type of surgery depends upon how widespread the cancer is when diagnosed (the cancer stage), as well as the presumed type and grade of cancer. The surgeon may remove one (unilateral oophorectomy) or both ovaries (bilateral oophorectomy), the fallopian tubes (salpingectomy), and the uterus (hysterectomy). For some very early tumors (stage 1, low grade or low-risk disease), only the involved ovary and fallopian tube will be removed (called a “unilateral salpingo-oophorectomy,” USO), especially in young females who wish to preserve their fertility.

In advanced malignancy, where complete resection is not feasible, as much tumor as possible is removed (debulking surgery). In cases where this type of surgery is successful (i.e., <1 cm in diameter of tumor is left behind [“optimal debulking” ]), the prognosis is improved compared to patients where large tumor masses (>1 cm in diameter) are left behind. Minimally invasive surgical techniques may facilitate the safe removal of very large (greater than 10 cm) tumors with fewer complications of surgery.

Chemotherapy has been a general standard of care for ovarian cancer for decades, although with highly variable protocols. Chemotherapy is used after surgery to treat any residual disease, if appropriate. This depends on the histology of the tumor; some kinds of tumor (particularly teratoma) are not sensitive to chemotherapy. In some cases, there may be reason to perform chemotherapy first, followed by surgery.

For patients with stage IIIC epithelial ovarian adenocarcinomas who have undergone successful optimal debulking, a recent clinical trial demonstrated that median survival time is significantly longer for patient receiving intraperitoneal (IP) chemotherapy. Patients in this clinical trial reported less compliance with IP chemotherapy and fewer than half of the patients received all six cycles of IP chemotherapy. Despite this high “drop-out” rate, the group as a whole (including the patients that didn't complete IP chemotherapy treatment) survived longer on average than patients who received intravenous chemotherapy alone.

Some specialists believe the toxicities and other complications of IP chemotherapy will be unnecessary with improved IV chemotherapy drugs currently being developed. Although IP chemotherapy has been recommended as a standard of care for the first-line treatment of ovarian cancer, the basis for this recommendation has been challenged.

Radiation therapy is not effective for advanced stages because when vital organs are in the radiation field, a high dose cannot be safely delivered. Radiation therapy is then commonly avoided in such stages as the vital organs may not be able to withstand the problems associated with these ovarian cancer treatments.

Ovarian cancer usually has a poor prognosis. It is disproportionately deadly because it lacks any clear early detection or screening test, meaning that most cases are not diagnosed until they have reached advanced stages. More than 60% of women presenting with this cancer already have stage III or stage IV cancer, when it has already spread beyond the ovaries. Ovarian cancers shed cells into the naturally occurring fluid within the abdominal cavity. These cells can then implant on other abdominal (peritoneal) structures, included the uterus, urinary bladder, bowel and the lining of the bowel wall omentum forming new tumor growths before cancer is even suspected. The five-year survival rate for all stages of ovarian cancer is 45.5%. For cases where a diagnosis is made early in the disease, when the cancer is still confined to the primary site, the five-year survival rate is 92.7%.

F. Brain Cancer

A brain tumor is an intracranial solid neoplasm, a tumor (defined as an abnormal growth of cells) within the brain or the central spinal canal. Brain tumors include all tumors inside the cranium or in the central spinal canal. They are created by an abnormal and uncontrolled cell division, normally either in the brain itself (neurons, glial cells (astrocytes, oligodendrocytes, ependymal cells, myelin-producing Schwann cells), lymphatic tissue, blood vessels), in the cranial nerves, in the brain envelopes (meninges), skull, pituitary and pineal gland, or spread from cancers primarily located in other organs (metastatic tumors).

Any brain tumor is inherently serious and life-threatening because of its invasive and infiltrative character in the limited space of the intracranial cavity. However, brain tumors (even malignant ones) are not invariably fatal. Brain tumors or intracranial neoplasms can be cancerous (malignant) or non-cancerous (benign); however, the definitions of malignant or to benign neoplasms differs from those commonly used in other types of cancerous or non-cancerous neoplasms in the body. Its threat level depends on the combination of factors like the type of tumor, its location, its size and its state of development. Because the brain is well protected by the skull, the early detection of a brain tumor only occurs when diagnostic tools are directed at the intracranial cavity. Usually detection occurs in advanced stages when the presence of the tumor has caused unexplained symptoms.

Primary (true) brain tumors are commonly located in the posterior cranial fossa in children and in the anterior two-thirds of the cerebral hemispheres in adults, although they can affect any part of the brain.

The prognosis of brain cancer varies based on the type of cancer. Medulloblastoma has a good prognosis with chemotherapy, radiotherapy, and surgical resection while glioblastoma multiforme has a median survival of only 12 months even with aggressive chemoradiotherapy and surgery. Brainstem gliomas have the poorest prognosis of any form of brain cancer, with most patients dying within one year, even with therapy that typically consists of radiation to the tumor along with corticosteroids. However, one type of brainstem glioma, a focal seems open to exceptional prognosis and long-term survival has frequently been reported.

Glioblastoma multiforme is the deadliest and most common form of malignant brain tumor. Even when aggressive multimodality therapy consisting of radiotherapy, chemotherapy, and surgical excision is used, median survival is only 12-17 months. Standard therapy for glioblastoma multiforme consists of maximal surgical resection of the tumor, followed by radiotherapy between two and four weeks after the surgical procedure to remove the cancer. This is followed by chemotherapy. Most patients with glioblastoma take a corticosteroid, typically dexamethasone, during their illness to palliate symptoms. Experimental treatments include gamma-knife radiosurgery, boron neutron capture therapy and gene transfer.

Oligodendroglioma is an incurable but slowly progressive malignant brain tumor. They can be treated with surgical resection, chemotherapy, and/or radiotherapy. For suspected low-grade oligodendrogliomas in select patients, some neuro-oncologists opt for a course of watchful waiting, with only symptomatic therapy. Tumors with the 1p/19q co-deletion have been found to be especially chemosensitive, and one source reports oligodendrogliomas to be among the most chemosensitive of human solid malignancies. A median survival of up to 16.7 years has been reported for low grade oligodendrogliomas.

Although there is no specific or singular clinical symptom or sign for any brain tumors, the presence of a combination of symptoms and the lack of corresponding clinical indications of infections or other causes can be an indicator to redirect diagnostic investigation towards the possibility of an intracranial neoplasm.

The diagnosis will often start with an interrogation of the patient to get a clear view of his medical antecedents, and his current symptoms. Clinical and laboratory investigations will serve to exclude infections as the cause of the symptoms. Examinations in this stage may include ophtamological, otolaryngological (or ENT) and/or electrophysiological exams. The use of electroencephalography (EEG) often plays a role in the diagnosis of brain tumors.

Swelling, or obstruction of the passage of cerebrospinal fluid (CSF) from the brain may cause (early) signs of increased intracranial pressure which translates clinically into headaches, vomiting, or an altered state of consciousness, and in children changes to the diameter of the skull and bulging of the fontanelles. More complex symptoms such as endocrine dysfunctions should alarm doctors not to exclude brain tumors.

A bilateral temporal visual field defect (due to compression of the optic chiasm) or dilatation of the pupil, and the occurrence of either slowly evolving or the sudden onset of focal neurologic symptoms, such as cognitive and behavioral impairment (including impaired judgment, memory loss, lack of recognition, spatial orientation disorders), personality or emotional changes, hemiparesis, hypoesthesia, aphasia, ataxia, visual field impairment, impaired sense of smell, impaired hearing, facial paralysis, double vision, or more severe symptoms such as tremors, paralysis on one side of the body hemiplegia, or (epileptic) seizures in a patient with a negative history for epilepsy, should raise the possibility of a brain tumor.

Imaging plays a central role in the diagnosis of brain tumors. Early imaging methods—invasive and sometimes dangerous—such as pneumoencephalography and cerebral angiography, have been abandoned in recent times in favor of non-invasive, high-resolution techniques, such as computed tomography (CT)-scans and especially magnetic resonance imaging (MRI). Neoplasms will often show as differently colored masses (also referred to as processes) in CT or MRI results.

Benign brain tumors often show up as hypodense (darker than brain tissue) mass lesions on cranial CT-scans. On MRI, they appear either hypo- (darker than brain tissue) or isointense (same intensity as brain tissue) on T1-weighted scans, or hyperintense (brighter than brain tissue) on T2-weighted MRI, although the appearance is variable.

Contrast agent uptake, sometimes in characteristic patterns, can be demonstrated on either CT or MRI-scans in most malignant primary and metastatic brain tumors. Perifocal edema, or pressure-areas, or where the brain tissue has been compressed by an invasive process also appears hyperintense on T2-weighted MRI might indicate the presence a diffuse neoplasm (unclear outline). This is because these tumors disrupt the normal functioning of the blood-brain barrier and lead to an increase in its permeability. However, it is not possible to diagnose high versus low grade gliomas based on enhancement pattern alone.

Glioblastoma multiforme and anaplastic astrocytoma have been associated with the genetic acute hepatic porphyrias (PCT, AIP, HCP and VP), including positive testing associated with drug refractory seizures. Unexplained complications associated with drug treatments with these tumors should alert physicians to an undiagnosed neurological porphyria.

The definitive diagnosis of brain tumor can only be confirmed by histological examination of tumor tissue samples obtained either by means of brain biopsy or open surgery. The histological examination is essential for determining the appropriate treatment and the correct prognosis. This examination, performed by a pathologist, typically has three stages: interoperative examination of fresh tissue, preliminary microscopic examination of prepared tissues, and followup examination of prepared tissues after immunohistochemical (IHC) staining or genetic analysis.

When a brain tumor is diagnosed, a medical team will be formed to assess the treatment options presented by the leading surgeon to the patient and his/her family. Given the location of primary solid neoplasms of the brain in most cases a “do-nothing” option is usually not presented. Neurosurgeons take the time to observe the evolution of the neoplasm before proposing a management plan to the patient and his/her relatives. These various types of treatment are available depending on neoplasm type and location and may be combined to give the best chances of survival: surgery: complete or partial resection of the tumor with the objective of removing as many tumor cells as possible; radiotherapy; and chemotherapy, with the aim of killing as many as possible of cancerous cells left behind after surgery and of putting remaining tumor cells into a non-dividing, sleeping state for as long as possible.

Survival rates in primary brain tumors depend on the type of tumor, age, functional status of the patient, the extent of surgical tumor removal and other factors specific to each case.

The primary and most desired course of action described in medical literature is surgical removal (resection) via craniotomy. Minimally invasive techniques are being studied but are far from being common practice. The prime remediating objective of surgery is to remove as many tumor cells as possible, with complete removal being the best outcome and cytoreduction (“debulking”) of the tumor otherwise. In some cases access to the tumor is impossible and impedes or prohibits surgery.

Many meningiomas, with the exception of some tumors located at the skull base, can be successfully removed surgically. Most pituitary adenomas can be removed surgically, often using a minimally invasive approach through the nasal cavity and skull base (trans-nasal, trans-sphenoidal approach). Large pituitary adenomas require a craniotomy (opening of the skull) for their removal. Radiotherapy, including stereotactic approaches, is reserved for inoperable cases.

Several current research studies aim to improve the surgical removal of brain tumors by labeling tumor cells with a chemical (5-aminolevulinic acid) that causes them to fluoresce. Post-operative radiotherapy and chemotherapy are integral parts of the therapeutic standard for malignant tumors. Radiotherapy may also be administered in cases of “low-grade” gliomas, when a significant tumor burden reduction could not be achieved surgically.

Any person undergoing brain surgery may suffer from epileptic seizures. Seizures can vary from absences to severe tonic-clonic attacks. Medication is prescribed and administered to minimize or eliminate the occurrence of seizures.

Multiple metastatic tumors are generally treated with radiotherapy and chemotherapy rather than surgery. The prognosis in such cases is determined by the primary tumor, but is generally poor.

The goal of radiation therapy is to selectively kill tumor cells while leaving normal brain tissue unharmed. In standard external beam radiation therapy, multiple treatments of standard-dose “fractions” of radiation are applied to the brain. This process is repeated for a total of 10 to 30 treatments, depending on the type of tumor. This additional treatment provides some patients with improved outcomes and longer survival rates.

Radiosurgery is a treatment method that uses computerized calculations to focus radiation at the site of the tumor while minimizing the radiation dose to the surrounding brain. Radiosurgery may be an adjunct to other treatments, or it may represent the primary treatment technique for some tumors.

Radiotherapy may be used following, or in some cases in place of, resection of the tumor. Forms of radiotherapy used for brain cancer include external beam radiation therapy, brachytherapy, and in more difficult cases, stereotactic radiosurgery, such as Gamma knife, Cyberknife or Novalis Tx radiosurgery.

Radiotherapy is the most common treatment for secondary brain tumors. The amount of radiotherapy depends on the size of the area of the brain affected by cancer. Conventional external beam ‘whole brain radiotherapy treatment’ (WBRT) or ‘whole brain irradiation’ may be suggested if there is a risk that other secondary tumors will develop in the future. Stereotactic radiotherapy is usually recommended in cases involving fewer than three small secondary brain tumors.

Patients undergoing chemotherapy are administered drugs designed to kill tumor cells. Although chemotherapy may improve overall survival in patients with the most malignant primary brain tumors, it does so in only about 20 percent of patients. Chemotherapy is often used in young children instead of radiation, as radiation may have negative effects on the developing brain. The decision to prescribe this treatment is based on a patient's overall health, type of tumor, and extent of the cancer. The toxicity and many side-effects of the drugs, and the uncertain outcome of chemotherapy in brain tumors puts this treatment further down the line of treatment options with surgery and radiation therapy preferred.

A shunt is used not as a cure but to relieve symptoms by reducing hydrocephalus caused by blockage of cerebrospinal fluid.

Researchers are presently investigating a number of promising new treatments including gene therapy, highly focused radiation therapy, immunotherapy and novel chemotherapies. A variety of new treatments are being made available on an investigational basis at centers specializing in brain tumor therapies.

G. Pancreatic Cancer

Pancreatic cancer arises when cells in the pancreas, a glandular organ behind the stomach, begin to multiply out of control and form a mass. These cancer cells have the ability to invade other parts of the body. There are a number of types of pancreatic cancer. The most common, pancreatic adenocarcinoma, accounts for about 85% of cases, and the term “pancreatic cancer” is sometimes used to refer only to that type. These adenocarcinomas start within the part of the pancreas which make digestive enzymes. Several other types of cancer, which collectively represent the majority of the non-adenocarcinomas, can also arise from these cells. One to two in every hundred cases of pancreatic cancer are neuroendocrine tumors, which arise from the hormone-producing cells of the pancreas. These are generally less aggressive than pancreatic adenocarcinoma.

Signs and symptoms of the most common form of pancreatic cancer may include yellow skin, abdominal or back pain, unexplained weight loss, light-colored stools, dark urine and loss of appetite. There are usually no symptoms in the disease's early stages, and symptoms that are specific enough to suspect pancreatic cancer typically do not develop until the disease has reached an advanced stage. By the time of diagnosis, pancreatic cancer has often spread to other parts of the body.

Pancreatic cancer rarely occurs before the age of 40, and more than half of cases of pancreatic adenocarcinoma occur in those over 70. Risk factors for pancreatic cancer include tobacco smoking, obesity, diabetes, and certain rare genetic conditions. About 25% of cases are linked to smoking, and 5-10% are linked to inherited genes, such as BRCA1. Pancreatic cancer is usually diagnosed by a combination of medical imaging techniques such as ultrasound or computed tomography, blood tests, and examination of tissue samples (biopsy). The disease is divided into stages, from early (stage I) to late (stage IV). Screening the general population has not been found to be effective.

The risk of developing pancreatic cancer is lower among non-smokers, and people who maintain a healthy weight and limit their consumption of red or processed meat. A smoker's chance of developing the disease decreases if they stop smoking, and almost returns to that of the rest of the population after 20 years. Pancreatic cancer can be treated with surgery, radiotherapy, chemotherapy, palliative care, or a combination of these. Treatment options are partly based on the cancer stage. Surgery is the only treatment that can cure the disease; it may also be done to try to improve quality of life without the potential for cure. Pain management and medications to improve digestion are sometimes needed. Early palliative care is recommended even for those receiving treatment that aims for a cure.

In 2012, pancreatic cancers of all types were the seventh most common cause of cancer deaths, resulting in 330,000 deaths globally. In the United States, pancreatic cancer is the fourth most common cause of deaths due to cancer. The disease occurs most often in the developed world, where about 70% of the new cases in 2012 originated. Pancreatic adenocarcinoma typically has a very poor prognosis: after diagnosis, 25% of people survive one year and 5% live for five years. For cancers diagnosed early, the five-year survival rate rises to about 20%. Neuroendocrine cancers have better outcomes; at five years from diagnosis, 65% of those diagnosed are living, though survival varies considerably depending on the type of tumor.

The many types of pancreatic cancer can be divided into two general groups. The vast majority of cases (about 99%) occur in the part of the pancreas which produces digestive enzymes, known as the exocrine component. There are several sub-types of exocrine pancreatic cancers, but their diagnosis and treatment have much in common. The small minority of cancers that arise in the hormone-producing (endocrine) tissue of the pancreas have different clinical characteristics. Both groups occur mainly (but not exclusively) in people over 40, and are slightly more common in men, but some rare sub-types mainly occur in women or children.

The exocrine group is dominated by pancreatic adenocarcinoma (variations of this name may add “invasive” and “ductal”), which is by far the most common type, representing about 85% of all pancreatic cancers. This is despite the fact that the tissue from which it arises—the pancreatic ductal epithelium—represents less than 10% of the pancreas by cell volume. This cancer originates in the ducts that carry certain hormones and enzymes away from the pancreas. About 60-70% of adenocarcinomas occur in the ‘head’ of the pancreas (see diagram, right).

The next most common type, acinar cell carcinoma of the pancreas, arises in the clusters of cells that produce these enzymes, and represents 5% of exocrine pancreas cancers. Like the ‘functioning’ endocrine cancers described below, acinar cell carcinomas may cause over-production of certain molecules, in this case digestive enzymes, which may cause symptoms such as skin rashes and joint pain.

Cystadenocarcinomas account for 1% of pancreatic cancers, and they have a better prognosis than the other exocrine types.

Pancreatoblastoma is a rare form, mostly occurring in childhood, and with a relatively good prognosis. Other exocrine cancers include adenosquamous carcinomas, signet ring cell carcinomas, hepatoid carcinomas, colloid carcinomas, undifferentiated carcinomas, and undifferentiated carcinomas with osteoclast-like giant cells. Solid pseudopapillary tumor is a rare low-grade neoplasm that mainly affects younger women, and generally has a very good prognosis.

Pancreatic mucinous cystic neoplasms are a broad group of pancreas tumors that have varying malignant potential. They are being detected at a greatly increased rate as CT scans become more powerful and common, and discussion continues as how best to assess and treat them, given that many are benign.

The small minority of tumors that arise elsewhere in the pancreas are mainly pancreatic neuroendocrine tumors (PanNETs). Neuroendocrine tumors (NETs) are a diverse group of benign or malignant tumors that arise from the body's neuroendocrine cells, which are responsible for integrating the nervous and endocrine systems. NETs can start in most organs of the body, including the pancreas, where the various malignant types are all considered to be rare. PanNETs are grouped into ‘functioning’ and ‘non-functioning’ types, depending on the degree to which they produce hormones. The functioning types secrete hormones such as insulin, gastrin, and glucagon into the bloodstream, often in large quantities, giving rise to serious symptoms such as low blood sugar, but also favoring relatively early detection. The most common functioning PanNETs are insulinomas and gastrinomas, named after the hormones they secrete. The non-functioning types do not secrete hormones in a sufficient quantity to give rise to overt clinical symptoms. For this reason, non-functioning PanNETs are often diagnosed only after the cancer has spread to other parts of the body.

As with other neuroendocrine tumors, the history of the terminology and classification of PanNETs is complex. PanNETs are sometimes called “islet cell cancers,” even though it is now known that they do not actually arise from islet cells as previously thought.

The symptoms of pancreatic adenocarcinoma do not usually appear in the disease's early stages, and are individually not distinctive to the disease. The symptoms at diagnosis vary according to the location of the cancer in the pancreas, which anatomists divide (from left to right on most diagrams) into the thick head, the neck, and the tapering body, ending in the tail.

Regardless of a tumour's location, the most common symptom is unexplained weight loss, which may be considerable. A large minority (between 35% and 47%) of people diagnosed with the disease will have had nausea, vomiting or a feeling of weakness.

Tumors in the head of the pancreas typically also cause jaundice, pain, loss of appetite, dark urine, and light-colored stools. Tumors in the body and tail typically also cause pain.

People sometimes have recent onset of atypical type 2 diabetes that is difficult to control, a history of recent but unexplained blood vessel inflammation caused by blood clots (thrombophlebitis) known as Trousseau sign, or a previous attack of pancreatitis. A doctor may suspect pancreatic cancer when the onset of diabetes in someone over 50-years-old is accompanied by typical symptoms such as unexplained weight loss, persistent abdominal or back pain, indigestion, vomiting, or fatty feces. Jaundice accompanied by a painlessly swollen gallbladder (known as Courvoisier's sign) may also raise suspicion, and can help differentiate pancreatic cancer from gallstones.

Medical imaging techniques, such as computed tomography (CT scan) and endoscopic ultrasound (EUS) are used both to confirm the diagnosis and to help decide whether the tumor can be surgically removed (its “resectability”). Magnetic resonance imaging and positron emission tomography may also be used, and magnetic resonance cholangiopancreatography may be useful in some cases. Abdominal ultrasound is less sensitive and will miss small tumors, but can identify cancers that have spread to the liver and build-up of fluid in the peritoneal cavity (ascites). It may be used for a quick and cheap first examination before other techniques.

A biopsy by fine needle aspiration, often guided by endoscopic ultrasound, may be used where there is uncertainty over the diagnosis, but a histologic diagnosis is not usually required for removal of the tumor by surgery to go ahead.

Liver function tests can show a combination of results indicative of bile duct obstruction (raised conjugated bilirubin, γ-glutamyl transpeptidase and alkaline phosphatase levels). CA19-9 (carbohydrate antigen 19.9) is a tumor marker that is frequently elevated in pancreatic cancer. However, it lacks sensitivity and specificity, not least because 5% of people lack the Lewis (a) antigen and cannot produce CA19-9. It has a sensitivity of 80% and specificity of 73% in for detecting pancreatic adenocarcinoma, and is used for following known cases rather than diagnosis.

The most common form of pancreatic cancer (adenocarcinoma) is typically characterized by moderately to poorly differentiated glandular structures on microscopic examination. There is typically considerable desmoplasia or formation of a dense fibrous stroma or structural tissue consisting of a range of cell types (including myofibroblasts, macrophages, lymphocytes and mast cells) and deposited material (such as type I collagen and hyaluronic acid). This creates a tumor microenvironment that is short of blood vessels (hypovascular) and so of oxygen (tumor hypoxia). It is thought that this prevents many chemotherapy drugs from reaching the tumor, as one factor making the cancer especially hard to treat.

A key assessment that is made after diagnosis is whether surgical removal of the tumor is possible (see Staging), as this is the only cure for this cancer. Whether or not surgical resection can be offered depends on how much the cancer has spread. The exact location of the tumor is also a significant factor, and CT can show how it relates to the major blood vessels passing close to the pancreas. The general health of the person must also be assessed, though age in itself is not an obstacle to surgery.

Chemotherapy and, to a lesser extent, radiotherapy are likely to be offered to most people, whether or not surgery is possible. Specialists advise that the management of pancreatic cancer should be in the hands of a multidisciplinary team including specialists in several aspects of oncology, and is therefore best conducted in larger centers.

Surgery with the intention of a cure is only possible in around one-fifth (20%) of new cases. Although CT scans help, in practice it can be difficult to determine whether the tumor can be fully removed (its “resectability”), and it may only become apparent during surgery that it is not possible to successfully remove the tumor without damaging other vital tissues. Whether or not surgical resection can be offered depends on various factors, including the precise extent of local anatomical adjacency to, or involvement of, the venous or arterial blood vessels, as well as surgical expertise and a careful consideration of projected post-operative recovery. The age of the person is not in itself a reason not to operate, but their general performance status needs to be adequate for a major operation.

One particular feature that is looked is the encouraging presence, or discouraging absence, of a clear layer or plane of fat giving a barrier between the tumor and the vessels. Traditionally, an assessment is made of the tumor's proximity to major venous or arterial vessels, in terms of “abutment” (defined as the tumor touching no more than half a blood vessel's circumference without any fat to separate it), “encasement” (when the tumor encloses most of the vessel's circumference), or full vessel involvement. A resection that includes encased sections of blood vessels may be possible in some cases, particularly if preliminary neoadjuvant therapy is feasible, using chemotherapy and/or radiotherapy.

Even when the operation appears to have been successful, cancerous cells are often found around the edges (“margins”) of the removed tissue, when a pathologist examines them microscopically (this will always be done), indicating the cancer has not been entirely removed. Furthermore, cancer stem cells are usually not evident microscopically, and if they are present they may continue to develop and spread. An exploratory laparoscopy (a small, camera-guided surgical procedure) may therefore be performed to gain a clearer idea of the outcome of a full operation.

For cancers involving the head of the pancreas, the Whipple procedure is the most commonly attempted curative surgical treatment. This is a major operation which involves removing the pancreatic head and the curve of the duodenum together (“pancreato-duodenectomy”), making a bypass for food from the stomach to the jejunum (“gastro-jejunostomy”) and attaching a loop of jejunum to the cystic duct to drain bile (“cholecysto-jejunostomy”). It can be performed only if the person is likely to survive major surgery and if the cancer is localized without invading local structures or metastasizing. It can therefore be performed only in a minority of cases. Cancers of the tail of the pancreas can be resected using a procedure known as a distal pancreatectomy, which often also entails removal of the spleen. Nowadays, this can often be done using minimally invasive surgery.

Although curative surgery no longer entails the very high death rates that occurred until the 1980s, a high proportion of people (about 30-45%) still have to be treated for post-operative sickness that is not caused by the cancer itself. The most common complication of surgery is difficulty in emptying the stomach. Certain more limited surgical procedures may also be used to ease symptoms. For instance, if the cancer is invading or compressing the duodenum or colon. In such cases, bypass surgery might overcome the obstruction and improve quality of life but is not intended as a cure.

After surgery, adjuvant chemotherapy with gemcitabine or 5-FU can be offered if the person is sufficiently fit, after a recovery period of one to two months. In people not suitable for curative surgery, chemotherapy may be used to extend life or improve its quality. Before surgery, neoadjuvant chemotherapy or chemoradiotherapy may be used in cases that are considered to be “borderline resectable” (see Staging) in order to reduce the cancer to a level where surgery could be beneficial. In other cases neoadjuvant therapy remains controversial, because it delays surgery.

Gemcitabine was approved by the United States Food and Drug Administration (FDA) in 1997, after a clinical trial reported improvements in quality of life and a 5-week improvement in median survival duration in people with advanced pancreatic cancer. This was the first chemotherapy drug approved by the FDA primarily for a nonsurvival clinical trial endpoint. Chemotherapy using gemcitabine alone was the standard for about a decade, as a number of trials testing it in combination with other drugs failed to demonstrate significantly better outcomes. However, the combination of gemcitabine with erlotinib was found to increase survival modestly, and erlotinib was licensed by the FDA for use in pancreatic cancer in 2005.

The FOLFIRINOX chemotherapy regimen using four drugs was found more effective than gemcitabine, but with substantial side effects, and is thus only suitable for people with good performance status. This is also true of protein-bound paclitaxel (nab-paclitaxel), which was licensed by the FDA in 2013 for use with gemcitabine in pancreas cancer. By the end of 2013, both FOLFIRINOX and nab-paclitaxel with gemcitabine were regarded as good choices for those able to tolerate the side-effects, and gemcitabine remained an effective option for those who were not. A head-to-head trial between the two new options is awaited, and trials investigating other variations continue. However, the changes of the last few years have only increased survival times by a few months. Clinical trials are often conducted for novel adjuvant therapies.

The role of radiotherapy as an auxiliary (adjuvant) treatment after potentially curative surgery has been controversial since the 1980s. The European Society for Medical Oncology recommends that adjuvant radiotherapy should only be used for people enrolled in clinical trials. However there is a continuing tendency for clinicians in the US to be more ready to use adjuvant radiotherapy than those in Europe. Many clinical trials have tested a variety of treatment combinations since the 1980s, but have failed to settle the matter conclusively.

Radiotherapy may form part of treatment to attempt to shrink a tumor to a resectable state, but its use on unresectable tumors remains controversial as there are conflicting results from clinical trials. The preliminary results of one trial, presented in 2013, “markedly reduced enthusiasm” for its use on locally advanced tumors.

H. Cervical Cancer

Cervical cancer is a cancer arising from the cervix. It is due to the abnormal growth of cells that have the ability to invade or spread to other parts of the body. Early on there are typically no symptoms. Later symptoms may include: abnormal vaginal bleeding, pelvic pain or pain during sexual intercourse. Bleeding after sex may be not serious; however, may also be due to cervical cancer.

Human papillomavirus (HPV) infection appears to be involved in the development of more than 90% of cases; most people who have had HPV infections, however, do not develop cervical cancer. Other risk factors include: smoking, a weak immune system, birth control pills, starting sex at a young age and having many sexual partners, but these are less important. Cervical cancer typically develops from precancerous changes over 10 to 20 years. There are a few types of cervical cancer. About 90% are squamous cell carcinomas, 10% are adenocarcinoma and a small number are other types. Diagnosis is typically by cervical screening followed by a biopsy. Medical imaging is then done to determine whether or not the cancer has spread.

HPV vaccines protect against two high risk strains of this family of viruses and may prevent up to 65 to 75% of cervical cancers. As there still is a risk of cancer, guidelines recommend continuing regular Pap smears. Other methods of prevention include: never having sex or having few sexual partners and the use of condoms. Cervical cancer screening using the Pap smear or acetic acid can identify precancerous changes which when treated can prevent the development of cancer. Treatment of cervical cancer may consist of some combination of surgery, chemotherapy and radiotherapy. Five year survival rates in the United States are 68%. Outcomes, however, depend very much on how early the cancer is detected.

Worldwide, cervical cancer is both the fourth most common cause of cancer and the fourth most common cause of death from cancer in women. In 2012, it was estimated that there were 528,000 cases of cervical cancer, and 266,000 deaths. This is about 8% of the total cases and total deaths from cancer. Approximately 70% of cervical cancers occur in developing countries. In low income countries it is the most common cause of cancer death. In developed countries, the widespread use of cervical screening programs has dramatically reduced rates of cervical cancer.

The early stages of cervical cancer may be completely free of symptoms. Vaginal bleeding, contact bleeding (one most common form being bleeding after sexual intercourse), or (rarely) a vaginal mass may indicate the presence of malignancy. Also, moderate pain during sexual intercourse and vaginal discharge are symptoms of cervical cancer. In advanced disease, metastases may be present in the abdomen, lungs or elsewhere.

Symptoms of advanced cervical cancer may include: loss of appetite, weight loss, fatigue, pelvic pain, back pain, leg pain, swollen legs, heavy bleeding from the vagina, bone fractures, and/or (rarely) leakage of urine or feces from the vagina.

Infection with some types of human papilloma virus (HPV) is the greatest risk factor for cervical cancer, followed by smoking. Other risk factors include human immunodeficiency virus. Not all of the causes of cervical cancer are known, however, and several other contributing factors have been implicated.

The pap smear can be used as a screening test, but is false negative in up to 50% of cases of cervical cancer. Confirmation of the diagnosis of cervical cancer or pre-cancer requires a biopsy of the cervix. This is often done through colposcopy, a magnified visual inspection of the cervix aided by using a dilute acetic acid (e.g., vinegar) solution to highlight abnormal cells on the surface of the cervix. Medical devices used for biopsy of the cervix include punch forceps, SpiraBrush CX, SoftBiopsy or Soft-ECC.

Colposcopic impression, the estimate of disease severity based on the visual inspection, forms part of the diagnosis.

Further diagnostic and treatment procedures are loop electrical excision procedure (LEEP) and conization, in which the inner lining of the cervix is removed to be examined pathologically. These are carried out if the biopsy confirms severe cervical intraepithelial neoplasia.

Often before the biopsy, the doctor asks for medical imaging to rule out other causes of woman's symptoms. Imaging modalities including ultrasound, CT scan and MRI have been used to different extent in order to look for alternating disease/spread of tumor/effect on adjacent structures. Typically they appear as heterogeneous mass in the cervix.

Cervical intraepithelial neoplasia, the potential precursor to cervical cancer, is often diagnosed on examination of cervical biopsies by a pathologist. For premalignant dysplastic changes, the CIN (cervical intraepithelial neoplasia) grading is used.

The naming and histologic classification of cervical carcinoma precursor lesions has changed many times over the 20th century. The World Health Organization classification system was descriptive of the lesions, naming them mild, moderate or severe dysplasia or carcinoma in situ (CIS). The term, Cervical Intraepithelial Neoplasia (CIN) was developed to place emphasis on the spectrum of abnormality in these lesions, and to help standardise treatment. It classifies mild dysplasia as CIN1, moderate dysplasia as CIN2, and severe dysplasia and CIS as CIN3. More recently, CIN2 and CIN3 have been combined into CIN2/3. These results are what a pathologist might report from a biopsy.

These should not be confused with the Bethesda System terms for Pap smear (cytopathology) results. Among the Bethesda results: Low-grade Squamous Intraepithelial Lesion (LSIL) and High-grade Squamous Intraepithelial Lesion (HSIL). An LSIL Pap may correspond to CIN1, and HSIL may correspond to CIN2 and CIN3, however they are results of different tests, and the Pap smear results need not match the histologic findings.

Histologic subtypes of invasive cervical carcinoma, include the following:
squamous cell carcinoma (about 80-85%)
adenocarcinoma (about 15% of cervical cancers in the UK)
adenosquamous carcinoma
small cell carcinoma
neuroendocrine tumour
glassy cell carcinoma
villoglandular adenocarcinoma
   Non-carcinoma malignancies which can rarely occur in the cervix include melanoma and lymphoma.

The treatment of cervical cancer varies worldwide, largely due to large variances in disease burden in developed and developing nations, access to surgeons skilled in radical pelvic surgery, and the emergence of “fertility sparing therapy” in developed nations. Because cervical cancers are radiosensitive, radiation may be used in all stages where surgical options do not exist.

Microinvasive cancer (stage IA) may be treated by hysterectomy (removal of the whole uterus including part of the vagina). For stage IA2, the lymph nodes are removed as well. Alternatives include local surgical procedures such as a loop electrical excision procedure (LEEP) or cone biopsy. For 1A1 disease, a cone biopsy (a.k.a. cervical conization) is considered curative.

If a cone biopsy does not produce clear margins (findings on biopsy showing that the tumor is surrounded by cancer free tissue, suggesting all of the tumor is removed), one more possible treatment option for women who want to preserve their fertility is a trachelectomy. This attempts to surgically remove the cancer while preserving the ovaries and uterus, providing for a more conservative operation than a hysterectomy. It is a viable option for those in stage I cervical cancer which has not spread; however, it is not yet considered a standard of care, as few doctors are skilled in this procedure. Even the most experienced surgeon cannot promise that a trachelectomy can be performed until after surgical microscopic examination, as the extent of the spread of cancer is unknown. If the surgeon is not able to microscopically confirm clear margins of cervical tissue once the woman is under general anesthesia in the operating room, a hysterectomy may still be needed. This can only be done during the same operation if the woman has given prior consent. Due to the possible risk of cancer spread to the lymph nodes in stage 1b cancers and some stage 1a cancers, the surgeon may also need to remove some lymph nodes from around the uterus for pathologic evaluation.

A radical trachelectomy can be performed abdominally or vaginally and there are conflicting opinions as to which is better. A radical abdominal trachelectomy with lymphadenectomy usually only requires a two to three day hospital stay, and most women recover very quickly (approximately six weeks). Complications are uncommon, although women who are able to conceive after surgery are susceptible to preterm labor and possible late miscarriage. It is generally recommended to wait at least one year before attempting to become pregnant after surgery. Recurrence in the residual cervix is very rare if the cancer has been cleared with the trachelectomy. Yet, it is recommended for women to practice vigilant prevention and follow up care including pap screenings/colposcopy, with biopsies of the remaining lower uterine segment as needed (every 3-4 months for at least 5 years) to monitor for any recurrence in addition to minimizing any new exposures to HPV through safe sex practices until one is actively trying to conceive.

Early stages (IB1 and IIA less than 4 cm) can be treated with radical hysterectomy with removal of the lymph nodes or radiation therapy. Radiation therapy is given as external beam radiotherapy to the pelvis and brachytherapy (internal radiation). Women treated with surgery who have high risk features found on pathologic examination are given radiation therapy with or without chemotherapy in order to reduce the risk of relapse.

Larger early stage tumors (IB2 and IIA more than 4 cm) may be treated with radiation therapy and cisplatin-based chemotherapy, hysterectomy (which then usually requires adjuvant radiation therapy), or cisplatin chemotherapy followed by hysterectomy. When cisplatin is present, it is thought to be the most active single agent in periodic diseases. Advanced stage tumors (IIB-IVA) are treated with radiation therapy and cisplatin-based chemotherapy.

On Jun. 15, 2006, the US Food and Drug Administration approved the use of a combination of two chemotherapy drugs, hycamtin and cisplatin for women with late-stage (IVB) cervical cancer treatment. Combination treatment has significant risk of neutropenia, anemia, and thrombocytopenia side effects. Hycamtin is manufactured by GlaxoSmithKline.

I. Head and Neck Cancer

Head and neck cancer is a cancer that starts in the lip, oral cavity (mouth), nasal cavity (inside the nose), paranasal sinuses, pharynx, and larynx. Most head and neck cancers are biologically similar. 90% of head and neck cancers are squamous cell carcinomas, so they are called head and neck squamous cell carcinomas (HNSCC). These cancers commonly originate from the mucosal lining (epithelium) of these regions. Head and neck cancers often spread to the lymph nodes of the neck, and this is often the first (and sometimes only) sign of the disease at the time of diagnosis.

Head and neck cancer is strongly associated with certain environmental and lifestyle risk factors, including tobacco smoking, alcohol consumption, UV light, particular chemicals used in certain workplaces, and certain strains of viruses, such as human papillomavirus. These cancers are frequently aggressive in their biologic behavior; patients with these types of cancer are at a higher risk of developing another cancer in the head and neck area. Head and neck cancer is highly curable if detected early, usually through surgery, but radiation therapy may also play an important role, while chemotherapy is often ineffective.

HNSCC is the sixth leading cancer by incidence worldwide. There are 0.5 million new cases a year worldwide. Two-thirds occur in industrialized nations. HNSCC usually develops in males in the 6th and 7th decade. The five-year survival rate of patients with HNSCC is about 40-50%.

Throat cancer usually begins with symptoms that seem harmless enough, like an enlarged lymph node on the outside of the neck, a sore throat or a hoarse sounding voice. However, in the case of throat cancer, these conditions may persist and become chronic. There may be a lump or a sore in the throat or neck that does not heal or go away. There may be difficult or painful swallowing. Speaking may become difficult. There may be a persistent earache. Other possible but less common symptoms include some numbness or paralysis of the face muscles.

Presenting symptoms include a mass in the neck, neck pain, bleeding from the mouth, sinus congestion, especially with nasopharyngeal carcinoma, bad breath, sore tongue, painless ulcer or sores in the mouth that do not heal, white, red or dark patches in the mouth that will not go away, ear ache, unusual bleeding or numbness in the mouth, lump in the lip, mouth or gums, enlarged lymph glands in the neck, slurring of speech (if the cancer is affecting the tongue), hoarse voice which persists for more than six weeks, sore throat which persists for more than six weeks, difficulty swallowing food, and a change in diet or weight loss.

A patient usually presents to the physician complaining of one or more of the above symptoms. The patient will typically undergo a needle biopsy of this lesion, and a histopathologic information is available, a multidisciplinary discussion of the optimal treatment strategy will be undertaken between the radiation oncologist, surgical oncologist, and medical oncologist.

Throat cancers are classified according to their histology or cell structure, and are commonly referred to by their location in the oral cavity and neck. This is because where the cancer appears in the throat affects the prognosis—some throat cancers are more aggressive than others depending upon their location. The stage at which the cancer is diagnosed is also a critical factor in the prognosis of throat cancer.

Squamous-cell carcinoma is a cancer of the squamous cell—a kind of epithelial cell found in both the skin and mucous membranes. It accounts for over 90% of all head and neck cancers, including more than 90% of throat cancer. Squamous cell carcinoma is most likely to appear in males over 40 years of age with a history of heavy alcohol use coupled with smoking.

The tumor marker Cyfra 21-1 may be useful in diagnosing squamous cell carcinoma of the head/neck.

Adenocarcinoma is a cancer of epithelial tissue that has glandular characteristics. Several head and neck cancers are adenocarcinomas (either of intestinal or non-intestinal cell-type).

Squamous cell cancers are common in the oral cavity, including the inner lip, tongue, floor of mouth, gingivae, and hard palate. Cancers of the oral cavity are strongly associated with tobacco use, especially use of chewing tobacco or “dip”, as well as heavy alcohol use. Cancers of this region, particularly the tongue, are more frequently treated with surgery than are other head and neck cancers.

Surgeries for oral cancers include Maxillectomy (can be done with or without orbital exenteration), Mandibulectomy (removal of the mandible or lower jaw or part of it), Glossectomy (tongue removal, can be total, hemi or partial), Radical neck dissection, Mohs procedure, Combinational e.g., glossectomy and laryngectomy done together.

The defect is typically covered/improved by using another part of the body and/or skin grafts and/or wearing a prosthesis.

Nasopharyngeal cancer arises in the nasopharynx, the region in which the nasal cavities and the Eustachian tubes connect with the upper part of the throat. While some nasopharyngeal cancers are biologically similar to the common HNSCC, “poorly differentiated” nasopharyngeal carcinoma is lymphoepithelioma, which is distinct in its epidemiology, biology, clinical behavior, and treatment, and is treated as a separate disease by many experts.

Oropharyngeal squamous cell carcinomas (OSCC) begins in the oropharynx, the middle part of the throat that includes the soft palate, the base of the tongue, and the tonsils. Squamous cell cancers of the tonsils are more strongly associated with human papillomavirus infection than are cancers of other regions of the head and neck. HPV-positive oropharyngeal cancer has a significantly more positive prognosis than HPV-negative disease (see HPV-positive oropharyngeal cancer for citations).

he hypopharynx includes the pyriform sinuses, the posterior pharyngeal wall, and the postcricoid area. Tumors of the hypopharynx frequently have an advanced stage at diagnosis, and have the most adverse prognoses of pharyngeal tumors. They tend to metastasize early due to the extensive lymphatic network around the larynx.

Laryngeal cancer begins in the larynx or “voice box.” Cancer may occur on the vocal folds themselves (“glottic” cancer), or on tissues above and below the true cords (“supraglottic” and “subglottic” cancers respectively). Laryngeal cancer is strongly associated with tobacco smoking.

Surgery can include laser excision of small vocal cord lesions, partial laryngectomy (removal of part of the larynx) or total laryngectomy (removal of the whole larynx). If the whole larynx has been removed the person is left with a permanent tracheostomy. Voice rehabilitation is such patients can be achieved through three important ways—esophageal speech, tracheoesophageal puncture or electrolarynx. One would likely require the help of intensive teaching and speech therapy and/or an electronic device.

Cancer of the trachea is a rare malignancy which can be biologically similar in many ways to head and neck cancer, and is sometimes classified as such.

Most tumors of the salivary glands differ from the common carcinomas of the head and neck in etiology, histopathology, clinical presentation, and therapy, Other uncommon tumors arising in the head and neck include teratomas, adenocarcinomas, adenoid cystic carcinomas, and mucoepidermoid carcinomas. Rarer still are melanomas and lymphomas of the upper aerodigestive tract.

After a histologic diagnosis has been established and tumor extent determined, the selection of appropriate treatment for a specific cancer depends on a complex array of variables, including tumor site, relative morbidity of various treatment options, patient performance and nutritional status, concomitant health problems, social and logistic factors, previous primary tumors, and patient preference. Treatment planning generally requires a multidisciplinary approach involving specialist surgeons and medical and radiation oncologists.

Several generalizations are useful in therapeutic decision making, but variations on these themes are numerous. Surgical resection and radiation therapy are the mainstays of treatment for most head and neck cancers and remain the standard of care in most cases. For small primary cancers without regional metastases (stage I or II), wide surgical excision alone or curative radiation therapy alone is used. More extensive primary tumors, or those with regional metastases (stage III or IV), planned combinations of pre- or postoperative radiation and complete surgical excision are generally used. More recently, as historical survival and control rates are recognized as less than satisfactory, there has been an emphasis on the use of various induction or concomitant chemotherapy regimens.

Many different treatments and therapies are used in the treatment of throat cancer. The type of treatment and therapies used are largely determined by the location of the cancer in the throat area and also the extent to which the cancer has spread at time of diagnosis. Patients' also have the right to decide whether or not they wish to consent to a particular treatment. For example, some may decide to not undergo radiation therapy which has serious side effects if it means they will be extending their lives by only a few months or so. Others may feel that the extra time is worth it and wish to pursue the treatments.

Surgery as a treatment is frequently used in most types of head and neck cancer. Usually the goal is to remove the cancerous cells entirely. This can be particularly tricky if the cancer is near the larynx and can result in the patient being unable to speak. Surgery is also commonly used to resect (remove) some or all of the cervical lymph nodes to prevent further spread of the disease.

CO₂ laser surgery is also another form of treatment. Transoral laser microsurgery allows surgeons to remove tumors from the voice box with no external incisions. It also allows access to tumors that are not reachable with robotic surgery. A microscope helps the surgeon clearly view the margins of the tumor, minimizing the amount of normal tissue removed or damaged during surgery. This technique helps give the patient as much speech and swallowing function as possible after surgery.

Radiation therapy is the most common form of treatment. There are different forms of radiation therapy, including 3D conformal radiation therapy, intensity-modulated radiation therapy, and brachytherapy, which are commonly used in the treatments of cancers of the head and neck. Most patients with head and neck cancer who are treated in the United States and Europe are treated with intensity-modulated radiation therapy using high energy photons.

Chemotherapy in throat cancer is not generally used to cure the cancer as such. Instead, it is used to provide an inhospitable environment for metastases so that they will not establish in other parts of the body. Typical chemotherapy agents are a combination of paclitaxel and carboplatin. Cetuximab is also used in the treatment of throat cancer.

Docetaxel-based chemotherapy has shown a very good response in locally advanced head and neck cancer. Taxotere is the only taxane approved by US FDA for Head and neck cancer, in combination with cisplatin and fluorouracil for the induction treatment of patients with inoperable, locally advanced squamous cell carcinoma of the head and neck.

While not specifically a chemotherapy, amifostine is often administered intravenously by a chemotherapy clinic prior to a patient's IMRT radiotherapy sessions. Amifostine protects the patient's gums and salivary glands from the effects of radiation.

Photodynamic therapy may have promise in treating mucosal dysplasia and small head and neck tumors. Amphinex is giving good results in early clinical trials for treatment of advanced head and neck cancer.

Targeted therapy, according to the National Cancer Institute, is “a type of treatment that uses drugs or other substances, such as monoclonal antibodies, to identify and attack specific cancer cells without harming normal cells.” Some targeted therapy used in squamous cell cancers of the head and neck include cetuximab, bevacizumab and erlotinib.

The best quality data are available for cetuximab since the 2006 publication of a randomized clinical trial comparing radiation treatment plus cetuximab versus radiation treatment alone. This study found that concurrent cetuximab and radiotherapy improves survival and locoregional disease control compared to radiotherapy alone, without a substantial increase in side effects, as would be expected with the concurrent chemoradiotherapy, which is the current gold standard treatment for advanced head and neck cancer. Whilst this study is of pivotal significance, interpretation is difficult since cetuximab-radiotherapy was not directly compared to chemoradiotherapy. The results of ongoing studies to clarify the role of cetuximab in this disease are awaited with interest.

Another study evaluated the impact of adding cetuximab to conventional chemotherapy (cisplatin) versus cisplatin alone. This study found no improvement in survival or disease-free survival with the addition of cetuximab to the conventional chemotherapy. A 2010 review concluded that the combination of cetuximab and platin/5-fluorouracil should be considered the current standard first-line regimen.

Gendicine is a gene therapy that employs an adenovirus to deliver the tumor suppressor gene p53 to cells. It was approved in China in 2003 for the treatment of head and neck squamous cell carcinoma.

Head and neck cancer clinical trials employing bevacizumab, an inhibitor of the angiogenesis receptor VEGF, were recruiting patients as of March 2007.

Erlotinib is an oral EGFR inhibitor, and was found in one Phase II clinical trial to retard disease progression. Scientific evidence for the effectiveness of erlotinib is otherwise lacking to this point. A clinical trial evaluating the use of erlotinib in metastatic head and neck cancer is recruiting patients as of March, 2007.

The mutational profile of HPV+ and HPV− head and neck cancer has been reported, further demonstrating that they are fundamentally distinct diseases.

### III. METHODS OF ASSESSING GENE/PROTEIN EXPRESSION AND STRUCTURE

A. Protein Based Methods

1. Antibody Conjugates

Antibodies may be linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radionuclides, antiviral agents, chelating agents, cytokines, growth factors, and oligo- or polynucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, such as biotin.

Antibody conjugates are generally preferred for use as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹⁵⁸I is often being preferred for use in certain embodiments, and technicium^{99m} and/or indium¹¹¹ are also often preferred due to their low energy and suitability for long range detection. Radioactively labeled monoclonal antibodies of the present disclosure may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated in the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds. The use of such labels is well known to those of skill in the art and are described, for example, in U.S. Pat. Nos. 3,817,837, 3,850,752, 3,939,350, 3,996,345, 4,277,437, 4,275,149 and 4,366,241.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter and Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or to tetrachloro-3α-6α-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region, have also been disclosed in the literature (O'Shannessy et al., 1987). This approach has been reported to produce diagnostically and therapeutically promising antibodies which are currently in clinical evaluation.

2. Antibody-Based Detection Methods

In still further embodiments, the present disclosure concerns immunodetection methods for binding, purifying, removing, quantifying and otherwise generally detecting Kub5/Hera, Artemis and/or CDK1. While such methods can be applied in a traditional sense, another use will be in quality control and monitoring of vaccine and other virus stocks, where antibodies according to the present disclosure can be used to assess the amount or integrity (i.e., long term stability) of H antigens in viruses. Alternatively, the methods may be used to screen various antibodies for appropriate/desired reactivity profiles.

Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. In particular, a competitive assay for the detection and quantitation of Kub5/Hera also is provided. The steps of various useful immunodetection methods have been described in the scientific literature, such as, e.g., Doolittle and Ben-Zeev (1999), Gulbis and Galand (1993), De Jager et al. (1993), and Nakamura et al. (1987). In general, the immunobinding methods include obtaining a sample suspected of containing Kub5/Hera-related cancers, and contacting the sample with a first antibody in accordance with the present disclosure, as the case may be, under conditions effective to allow the formation of immunocomplexes.

The immunobinding methods include methods for detecting and quantifying the amount of Kub5/Hera or related fragments in a sample and the detection and quantification of any immune complexes formed during the binding process. Here, one would obtain a sample suspected of containing Kub5/Hera-related cancer cells, and contact the sample with an antibody that binds Kub5/Hera or fragments thereof, followed by detecting and quantifying the amount of immune complexes formed under the specific conditions. In terms of antigen detection, the biological sample analyzed may be any sample that is suspected of containing Kub5/Hera-related cancers, such as a tissue section or specimen, a homogenized tissue extract, a biological fluid, including blood and serum, or a secretion, such as feces or urine.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to Kub5/Hera. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody that has binding affinity for the antibody, is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first biotinylated antibody is used to detect the target antigen, and a second antibody is then used to detect the biotin attached to the complexed biotin. In that method, the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

ELISAs.

Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, the antibodies of the disclosure are immobilized onto a selected surface exhibiting protein affinity, such as a well in a polystyrene microtiter plate. Then, a test composition suspected of containing the Kub5/Hera-related cancer cells is added to the wells. After binding and washing to remove non-specifically bound immune complexes, the bound antigen may be detected. Detection may be achieved by the addition of another anti-Kub5/Hera antibody that is linked to a detectable label. This type of ELISA is a simple “sandwich ELISA.” Detection may also be achieved by the addition of a second anti-Kub5/Hera antibody, followed by the addition of a third antibody that has binding affinity for the second antibody, with the third antibody being linked to a detectable label.

In another exemplary ELISA, the samples suspected of containing the Kub5/Hera-related cancer cells are immobilized onto the well surface and then contacted with the anti-Kub5/Hera antibodies of the disclosure. After binding and washing to remove non-specifically bound immune complexes, the bound anti-Kub5/Hera antibodies are detected. Where the initial anti-Kub5/Hera antibodies are linked to a detectable label, the immune complexes may be detected directly. Again, the immune complexes may be detected using a second antibody that has binding affinity for the first anti-Kub5/Hera antibody, with the second antibody being linked to a detectable label.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

Western Blot.

The Western blot (alternatively, protein immunoblot) is an analytical technique used to detect specific proteins in a given sample of tissue homogenate or extract. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein.

Samples may be taken from whole tissue or from cell culture. In most cases, solid tissues are first broken down mechanically using a blender (for larger sample volumes), using a homogenizer (smaller volumes), or by sonication. Cells may also be broken open by one of the above mechanical methods. However, it should be noted that bacteria, virus or environmental samples can be the source of protein and thus Western blotting is not restricted to cellular studies only. Assorted detergents, salts, and buffers may be employed to encourage lysis of cells and to solubilize proteins. Protease and phosphatase inhibitors are often added to prevent the digestion of the sample by its own enzymes. Tissue preparation is often done at cold temperatures to avoid protein denaturing.

The proteins of the sample are separated using gel electrophoresis. Separation of proteins may be by isoelectric point (pI), molecular weight, electric charge, or a combination of these factors. The nature of the separation depends on the treatment of the sample and the nature of the gel. This is a very useful way to determine a protein. It is also possible to use a two-dimensional (2-D) gel which spreads the proteins from a single sample out in two dimensions. Proteins are separated according to isoelectric point (pH at which they have neutral net charge) in the first dimension, and according to their molecular weight in the second dimension.

In order to make the proteins accessible to antibody detection, they are moved from within the gel onto a membrane made of nitrocellulose or polyvinylidene difluoride (PVDF). The membrane is placed on top of the gel, and a stack of filter papers placed on top of that. The entire stack is placed in a buffer solution which moves up the paper by capillary action, bringing the proteins with it. Another method for transferring the proteins is called to electroblotting and uses an electric current to pull proteins from the gel into the PVDF or nitrocellulose membrane. The proteins move from within the gel onto the membrane while maintaining the organization they had within the gel. As a result of this blotting process, the proteins are exposed on a thin surface layer for detection (see below). Both varieties of membrane are chosen for their non-specific protein binding properties (i.e., binds all proteins equally well). Protein binding is based upon hydrophobic interactions, as well as charged interactions between the membrane and protein. Nitrocellulose membranes are cheaper than PVDF, but are far more fragile and do not stand up well to repeated probings. The uniformity and overall effectiveness of transfer of protein from the gel to the membrane can be checked by staining the membrane with Coomassie Brilliant Blue or Ponceau S dyes. Once transferred, proteins are detected using labeled primary antibodies, or unlabeled primary antibodies followed by indirect detection using labeled protein A or secondary labeled antibodies binding to the Fc region of the primary antibodies.

Immunohistochemistry.

The antibodies of the present disclosure may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections from the capsule. Alternatively, whole frozen tissue samples may be used for serial section cuttings.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections. Again, whole tissue samples may be substituted.

Immunodetection Kits.

In still further embodiments, the present disclosure concerns immunodetection kits for use with the immunodetection methods described above. As the antibodies may be used to detect Kub5/Hera, the antibodies may be included in the kit. The immunodetection kits will thus comprise, in suitable container means, a first antibody that binds to Kub5/Hera, and optionally an immunodetection reagent.

In certain embodiments, the antibody may be pre-bound to a solid support, such as a column matrix and/or well of a microtitre plate. The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and all such labels may be employed in connection with the present disclosure.

The kits may further comprise a suitably aliquoted composition of Kub5/Hera, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the antibody may be placed, or preferably, suitably aliquoted. The kits of the present disclosure will also typically include a means for containing the antibody, antigen, and any other reagent containers in close confinement for commercial sale. Such containers may include injection or blow-molded plastic containers into which the desired vials are retained.

3. Liquid Chromatography-Mass Spectrometry

Liquid chromatography-mass spectrometry (LC-MS, or alternatively HPLC-MS) is an analytical chemistry technique that combines the physical separation capabilities of liquid chromatography (or HPLC) with the mass analysis capabilities of mass spectrometry (MS). LC-MS is a powerful technique that has very high sensitivity and selectivity and so is useful in many applications. Its application is oriented towards the separation, general detection and potential identification of chemicals of particular masses in the presence of other chemicals (i.e., in complex mixtures), e.g., natural products from natural-products extracts, and pure substances from mixtures of chemical intermediates. Preparative LC-MS systems can be used for rapid mass-directed purification of specific substances from such mixtures that are important in basic research, and pharmaceutical, agrochemical, food, and other industries.

Present day liquid chromatography generally utilizes very small particles packed and operating at relatively high pressure, and is referred to as high performance liquid chromatography (HPLC); modern LC-MS methods use HPLC instrumentation, essentially exclusively, for sample introduction. In HPLC, the sample is forced by a liquid at high pressure (the mobile phase) through a column that is packed with a stationary phase generally composed of irregularly or spherically shaped particles chosen or derivatized to accomplish particular types of separations. HPLC methods are historically divided into two different sub-classes based on stationary phases and the corresponding required polarity of the mobile phase. Use of octadecylsilyl (C18) and related organic-modified particles as stationary phase with pure or pH-adjusted water-organic mixtures such as water-acetonitrile and water-methanol are used in techniques termed reversed phase liquid chromatography (RP-LC). Use of materials such as silica gel as stationary phase with neat or mixed organic mixtures are used in techniques termed normal phase liquid chromatography (NP-LC). RP-LC is most often used as the means to introduce samples into the MS, in LC-MS instrumentation.

Mass spectrometry (MS) is an analytical technique that measures the mass-to-charge ratio of charged particles. It is used for determining masses of particles, for determining the elemental composition of a sample or molecule, and for elucidating the chemical structures of molecules, such as peptides and other chemical compounds. MS works by ionizing chemical compounds to generate charged molecules or molecule fragments and measuring their mass-to-charge ratios. (Morales et al., 2014). In a typical MS procedure, a sample is loaded onto the MS instrument and undergoes vaporization. The components of the sample are ionized by one of a variety of methods (e.g., by impacting them with an electron beam), which results in the formation of charged particles (ions). The ions are separated according to their mass-to-charge ratio in an analyzer by electromagnetic fields. The ions are detected, usually by a quantitative method. The ion signal is processed into mass spectra.

Additionally, MS instruments consist of three modules. An ion source, which can convert gas phase sample molecules into ions (or, in the case of electrospray ionization, move ions that exist in solution into the gas phase). A mass analyzer, which sorts the ions by their masses by applying electromagnetic fields. A detector, which measures the value of an indicator quantity and thus provides data for calculating the abundances of each ion present

The technique has both qualitative and quantitative uses. These include identifying unknown compounds, determining the isotopic composition of elements in a molecule, and determining the structure of a compound by observing its fragmentation. Other uses include quantifying the amount of a compound in a sample or studying the fundamentals of gas phase ion chemistry (the chemistry of ions and neutrals in a vacuum). MS is now in very common use in analytical laboratories that study physical, chemical, or biological properties of a great variety of compounds.

LC-MS is very commonly used in pharmacokinetic studies of pharmaceuticals and is thus the most frequently used technique in the field of bioanalysis. These studies give information about how quickly a drug will be cleared from the hepatic blood flow, and organs of the body. MS is used for this due to high sensitivity and exceptional specificity compared to UV (as long as the analyte can be suitably ionised), and short analysis time.

The major advantage MS has is the use of tandem MS-MS. The detector may be programmed to select certain ions to fragment. The process is essentially a selection technique, but is in fact more complex. The measured quantity is the sum of molecule fragments chosen by the operator. As long as there are no interferences or ion suppression, the LC separation can be quite quick.

LC-MS is also used in proteomics where again components of a complex mixture must be detected and identified in some manner. The bottom-up proteomics LC-MS approach to proteomics generally involves protease digestion and denaturation (usually trypsin as a protease, urea to denature tertiary structure and iodoacetamide to cap cysteine residues) followed by LC-MS with peptide mass fingerprinting or LC-MS/MS (tandem MS) to derive sequence of individual peptides. LC-MS/MS is most commonly used for proteomic analysis of complex samples where peptide masses may overlap even with a high-resolution mass spectrometer. Samples of complex biological fluids like human serum may be run in a modern LC-MS/MS system and result in over 1000 proteins being identified, provided that the sample was first separated on an SDS-PAGE gel or HPLC-SCX.

Profiling of secondary metabolites in plants or food like phenolics can be achieved with liquid chromatography-mass spectrometry.

LC-MS is frequently used in drug development at many different stages including peptide mapping, glycoprotein mapping, natural products dereplication, bioaffinity screening, in vivo drug screening, metabolic stability screening, metabolite identification, impurity identification, quantitative bioanalysis, and quality control.

B. Nucleic Acid-Based Analytics

1. SNP Analysis

The methods described herein include determining the identity, e.g., the specific nucleotide, presence or absence, of a SNP. The SNPs may be a gain of function mutation, a loss of function mutation, or have no effect. It is within the skill of those in the field to ascertain whether a mutation adds, detracts or has no change on the activity of a molecule examined, e.g., KUB/HERA, Artemis or CDK1.

Samples that are suitable for use in the methods described herein contain genetic material, e.g., genomic DNA (gDNA). Genomic DNA is typically extracted from biological samples. The sample itself will typically include a tumor biopsy removed from the subject. Methods and reagents are known in the art for obtaining, processing, and analyzing samples. In some embodiments, the sample is obtained with the assistance of a health care provider, e.g., to draw blood. In some embodiments, the sample is obtained without the assistance of a health care provider, e.g., where the sample is obtained non-invasively, such as a sample comprising buccal cells that is obtained using a buccal swab or brush, or a mouthwash sample.

In some cases, a biological sample may be processed for DNA isolation. For example, DNA in a cell or tissue sample can be separated from other components of the sample. Cells can be harvested from a biological sample using standard techniques known in the art. For example, cells can be harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract DNA, e.g., gDNA. See, e.g., Ausubel et al. (2003). The sample can be concentrated and/or purified to isolate DNA. All samples obtained from a subject, including those subjected to any sort of further processing, are considered to be obtained from the subject. Routine methods can be used to extract genomic DNA from a biological sample, including, for example, phenol extraction. Alternatively, genomic DNA can be extracted with kits such as the QIAamp® Tissue Kit (Qiagen, Chatsworth, Calif.) and the Wizard® Genomic DNA purification kit (Promega). Non-limiting examples of sources of samples include urine, blood, and tissue.

The presence or absence of the SNP can be determined using methods known in the art. For example, gel electrophoresis, capillary electrophoresis, size exclusion chromatography, sequencing, and/or arrays can be used to detect the presence or absence of specific response alleles. Amplification of nucleic acids, where desirable, can be accomplished using methods known in the art, e.g., PCR. In one example, a sample (e.g., a sample comprising genomic DNA), is obtained from a subject. The DNA in the sample is then examined to determine the identity of an allele as described herein, i.e., by determining the identity of one or more alleles associated with a selected response. The identity of an allele can be determined by any method described herein, e.g., by sequencing or by hybridization of the gene in the genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a particular polymorphic variant.

Other methods of nucleic acid analysis can include direct manual sequencing (Church and Gilbert, 1988; Sanger et al., 1977; U.S. Pat. No. 5,288,644); automated fluorescent sequencing; single-stranded conformation polymorphism assays (SSCP) (Schafer et al., 1995); clamped denaturing gel electrophoresis (CDGE); two-dimensional gel electrophoresis (2DGE or TDGE); conformational sensitive gel electrophoresis (CSGE); denaturing gradient gel electrophoresis (DGGE) (Sheffield et al., 1989); denaturing high performance liquid chromatography (DHPLC, Underhill et al., 1997); infrared matrix-assisted laser desorption/ionization (IR-MALDI) mass spectrometry (WO 99/57318); mobility shift analysis (Orita et al., 1989); restriction enzyme analysis (Flavell et al., 1978; Geever et al., 1981); quantitative real-time PCR (Raca et al., 2004); heteroduplex analysis; chemical mismatch cleavage (CMC) (Cotton et al., 1985); RNase protection assays (Myers et al., 1985); use of polypeptides that recognize nucleotide mismatches, e.g., E. coli mutS protein; allele-specific PCR, and combinations of such methods. See, e.g., U.S. Patent Publication No. 2004/0014095, which is incorporated herein by reference in its entirety.

Sequence analysis can also be used to detect specific polymorphic variants. For example, polymorphic variants can be detected by sequencing exons, introns, 5′ untranslated sequences, or 3′ untranslated sequences. A sample comprising DNA or RNA is obtained from the subject. PCR or other appropriate methods can be used to amplify a portion encompassing the polymorphic site, if desired. The sequence is then ascertained, using any standard method, and the presence of a polymorphic variant is determined. Real-time pyrophosphate DNA sequencing is yet another approach to detection of polymorphisms and polymorphic variants (Alderbom et al., 2000). Additional methods include, for example, PCR amplification in combination with denaturing high performance liquid chromatography (dHPLC) (Underhill et al., 1997).

PCR refers to procedures in which target nucleic acid (e.g., genomic DNA) is amplified in a manner similar to that described in U.S. Pat. No. 4,683,195, and subsequent modifications of the procedure described therein. Generally, sequence information from the ends of the region of interest or beyond are used to design oligonucleotide primers that are identical or similar in sequence to opposite strands of a potential template to be amplified. See e.g., PCR Primer: A Laboratory Manual, Dieffenbach and Dveksler, (Eds.); McPherson et al., 2000; Mattila et al., 1991; Eckert et al., 1991; PCR (eds. McPherson et al., IRL Press, Oxford); and U.S. Pat. No. 4,683,202. Other amplification methods that may be employed include the ligase chain reaction (LCR) (Wu and Wallace, 1989; Landegren et al., 1988), transcription amplification (Kwoh et al., 1989), self-sustained sequence replication (Guatelli et al., 1990), and nucleic acid based sequence amplification (NASBA). Guidelines for selecting primers for PCR amplification are well known in the art. See, e.g., McPherson et al. (2000). A variety of computer programs for designing primers are available, e.g., ‘Oligo’ (National Biosciences, Inc, Plymouth Minn.), MacVector (Kodak/IBI), and the GCG suite of sequence analysis programs (Genetics Computer Group, Madison, Wis. 53711).

In some cases, PCR conditions and primers can be developed that amplify a product only when the variant allele is present or only when the wild type allele is present (MSPCR or allele-specific PCR). For example, patient DNA and a control can be amplified separately using either a wild-type primer or a primer specific for the variant allele. Each set of reactions is then examined for the presence of amplification products using standard methods to visualize the DNA. For example, the reactions can be electrophoresed through an agarose gel and the DNA visualized by staining with ethidium bromide or other DNA intercalating dye. In DNA samples from heterozygous patients, reaction products would be detected in each reaction.

In some embodiments, a peptide nucleic acid (PNA) probe can be used instead of a nucleic acid probe in the hybridization methods described above. PNA is a DNA mimetic with a peptide-like, inorganic backbone, e.g., N-(2-aminoethyl)glycine units, with an organic base (A, G, C, T or U) attached to the glycine nitrogen via a methylene carbonyl linker (see, e.g., Nielsen et al., 1994). The PNA probe can be designed to specifically hybridize to a nucleic acid comprising a polymorphic variant.

In some cases, allele-specific oligonucleotides can also be used to detect the presence of a polymorphic variant. For example, polymorphic variants can be detected by performing allele-specific hybridization or allele-specific restriction digests. Allele specific hybridization is an example of a method that can be used to detect sequence variants, including complete genotypes of a subject (e.g., a mammal such as a human). See Stoneking et al. (1991); Prince et al. (2001). An “allele-specific oligonucleotide” (also referred to herein as an “allele-specific oligonucleotide probe”) is an oligonucleotide that is specific for particular a polymorphism can be prepared using standard methods (see, Ausubel et al., 2003). Allele-specific oligonucleotide probes typically can be approximately 10-50 base pairs, preferably approximately 15-30 base pairs, that specifically hybridizes to a nucleic acid region that contains a polymorphism. Hybridization conditions are selected such that a nucleic acid probe can specifically bind to the sequence of interest, e.g., the variant nucleic acid sequence. Such hybridizations typically are performed under high stringency as some sequence variants include only a single nucleotide difference. In some cases, dot-blot hybridization of amplified oligonucleotides with allele-specific oligonucleotide (ASO) probes can be performed. See, for example, Saiki et al. (1986).

In some embodiments, allele-specific restriction digest analysis can be used to detect the existence of a polymorphic variant of a polymorphism, if alternate polymorphic variants of the polymorphism result in the creation or elimination of a restriction site. Allele-specific restriction digests can be performed in the following manner. A sample containing genomic DNA is obtained from the individual and genomic DNA is isolated for analysis. For nucleotide sequence variants that introduce a restriction site, restriction digest with the particular restriction enzyme can differentiate the alleles. In some cases, polymerase chain reaction (PCR) can be used to amplify a region comprising the polymorphic site, and restriction fragment length polymorphism analysis is conducted (see, Ausubel et al., 2003). The digestion pattern of the relevant DNA fragment indicates the presence or absence of a particular polymorphic variant of the polymorphism and is therefore indicative of the subject's response allele. For sequence variants that do not alter a common restriction site, mutagenic primers can be designed that introduce a restriction site when the variant allele is present or when the wild type allele is present. For example, a portion of a nucleic acid can be amplified using the mutagenic primer and a wild type primer, followed by digest with the appropriate restriction endonuclease.

In some embodiments, fluorescence polarization template-directed dye-terminator incorporation (FP-TDI) is used to determine which of multiple polymorphic variants of a polymorphism is present in a subject (Chen et al., 1999). Rather than involving use of allele-specific probes or primers, this method employs primers that terminate adjacent to a polymorphic site, so that extension of the primer by a single nucleotide results in incorporation of a nucleotide complementary to the polymorphic variant at the polymorphic site.

In some cases, DNA containing an amplified portion may be dot-blotted, using standard methods (see Ausubel et al., 2003), and the blot contacted with the oligonucleotide probe. The presence of specific hybridization of the probe to the DNA is then detected. Specific hybridization of an allele-specific oligonucleotide probe (specific for a polymorphic variant indicative of a predicted response to a method of treating an SSD) to DNA from the subject is indicative of a subject's response allele.

The methods can include determining the genotype of a subject with respect to both copies of the polymorphic site present in the genome (i.e., both alleles). For example, the complete genotype may be characterized as −/−, as −/+, or as +/+, where a minus sign indicates the presence of the reference or wild type sequence at the polymorphic site, and the plus sign indicates the presence of a polymorphic variant other than the reference sequence. If multiple polymorphic variants exist at a site, this can be appropriately indicated by specifying which ones are present in the subject. Any of the detection means described herein can be used to determine the genotype of a subject with respect to one or both copies of the polymorphism present in the subject's genome.

Methods of nucleic acid analysis to detect polymorphisms and/or polymorphic variants can include, e.g., microarray analysis. Hybridization methods, such as Southern analysis, Northern analysis, or in situ hybridizations, can also be used (see, Ausubel et al., 2003). To detect microdeletions, fluorescence in situ hybridization (FISH) using DNA probes that are directed to a putatively deleted region in a chromosome can be used. For example, probes that detect all or a part of a microsatellite marker can be used to detect microdeletions in the region that contains that marker.

In some embodiments, it is desirable to employ methods that can detect the presence of multiple polymorphisms (e.g., polymorphic variants at a plurality of polymorphic sites) in parallel or substantially simultaneously. Oligonucleotide arrays represent one suitable means for doing so. Other methods, including methods in which reactions (e.g., amplification, hybridization) are performed in individual vessels, e.g., within individual wells of a multi-well plate or other vessel may also be performed so as to detect the presence of multiple polymorphic variants (e.g., polymorphic variants at a plurality of polymorphic sites) in parallel or substantially simultaneously according to the methods provided herein.

Nucleic acid probes can be used to detect and/or quantify the presence of a particular target nucleic acid sequence within a sample of nucleic acid sequences, e.g., as hybridization probes, or to amplify a particular target sequence within a sample, e.g., as a primer. Probes have a complimentary nucleic acid sequence that selectively hybridizes to the target nucleic acid sequence. In order for a probe to hybridize to a target sequence, the hybridization probe must have sufficient identity with the target sequence, i.e., at least 70% (e.g., 80%, 90%, 95%, 98% or more) identity to the target sequence. The probe sequence must also be sufficiently long so that the probe exhibits selectivity for the target sequence over non-target sequences. For example, the probe will be at least 20 (e.g., 25, 30, 35, 50, 100, 200, 300, 400, 500, 600, 700, 800, 900 or more) nucleotides in length. In some embodiments, the probes are not more than 30, 50, 100, 200, 300, 500, 750, or 1000 nucleotides in length. Probes are typically about 20 to about 1×10⁶ nucleotides in length. Probes include primers, which generally refers to a single-stranded oligonucleotide probe that can act as a point of initiation of template-directed DNA synthesis using methods such as PCR (polymerase chain reaction), LCR (ligase chain reaction), etc., for amplification of a target sequence.

The probe can be a test probe such as a probe that can be used to detect polymorphisms in a region described herein (e.g., an allele associated with treatment response as described herein). In some embodiments, the probe can bind to another marker sequence associated with SZ, SPD, or SD as described herein or known in the art.

Control probes can also be used. For example, a probe that binds a less variable sequence, e.g., repetitive DNA associated with a centromere of a chromosome, can be used as a control. Probes that hybridize with various centromeric DNA and locus-specific DNA are available commercially, for example, from Vysis, Inc. (Downers Grove, Ill.), Molecular Probes, Inc. (Eugene, Oreg.), or from Cytocell (Oxfordshire, UK). Probe sets are available commercially such from Applied Biosystems, e.g., the Assays-on-Demand SNP kits Alternatively, probes can be synthesized, e.g., chemically or in vitro, or made from chromosomal or genomic DNA through standard techniques. For example, sources of DNA that can be used include genomic DNA, cloned DNA sequences, somatic cell hybrids that contain one, or a part of one, human chromosome along with the normal chromosome complement of the host, and chromosomes purified by flow cytometry or microdissection. The region of interest can be isolated through cloning, or by site-specific amplification via the polymerase chain reaction (PCR). See, for example, Nath and Johnson, (1998); Wheeless et al., (1994); U.S. Pat. No. 5,491,224.

In some embodiments, the probes are labeled, e.g., by direct labeling, with a fluorophore, an organic molecule that fluoresces after absorbing light of lower wavelength/higher energy. A directly labeled fluorophore allows the probe to be visualized without a secondary detection molecule. After covalently attaching a fluorophore to a nucleotide, the nucleotide can be directly incorporated into the probe with standard techniques such as nick translation, random priming, and PCR labeling. Alternatively, deoxycytidine nucleotides within the probe can be transaminated with a linker. The fluorophore then is covalently attached to the transaminated deoxycytidine nucleotides. See, e.g., U.S. Pat. No. 5,491,224.

Fluorophores of different colors can be chosen such that each probe in a set can be distinctly visualized. For example, a combination of the following fluorophores can be used: 7-amino-4-methylcoumarin-3-acetic acid (AMCA), TEXAS RED™ (Molecular Probes, Inc., Eugene, Oreg.), 5-(and -6)-carboxy-X-rhodamine, lissamine rhodamine B, 5-(and -6)-carboxyfluorescein, fluorescein-5-isothiocyanate (FITC), 7-diethylaminocoumarin-3-carboxylic acid, tetramethylrhodamine-5-(and -6)-isothiocyanate, 5-(and -6)-carboxytetramethylrhodamine, 7-hydroxycoumarin-3-carboxylic acid, 6-[fluorescein 5-(and -6)-carboxamido]hexanoic acid, N-(4,4-difluoro-5,7-dimethyl-4-bora-3a,4a diaza-3-indacenepropionic acid, eosin-5-isothiocyanate, erythrosin-5-isothiocyanate, and CASCADE™ blue acetylazide (Molecular Probes, Inc., Eugene, Oreg.). Fluorescently labeled probes can be viewed with a fluorescence microscope and an appropriate filter for each fluorophore, or by using dual or triple band-pass filter sets to observe multiple fluorophores. See, for example, U.S. Pat. No. 5,776,688. Alternatively, techniques such as flow cytometry can be used to examine the hybridization pattern of the probes. Fluorescence-based arrays are also known in the art.

In other embodiments, the probes can be indirectly labeled with, e.g., biotin or digoxygenin, or labeled with radioactive isotopes such as ³²P and ³H. For example, a probe indirectly labeled with biotin can be detected by avidin conjugated to a detectable marker. For example, avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

2. Comparative Genomic Hybridization

Comparative genomic hybridization is a molecular cytogenetic method for analysing copy number variations (CNVs) relative to ploidy level in the DNA of a test sample compared to a reference sample, without the need for culturing cells. The aim of this technique is to quickly and efficiently compare two genomic DNA samples arising from two sources, which are most often closely related, because it is suspected that they contain differences in terms of either gains or losses of either whole chromosomes or subchromosomal regions (a portion of a whole chromosome). This technique was originally developed for the evaluation of the differences between the chromosomal complements of solid tumor and normal tissue, and has an improved resolution of 5-10 megabases compared to the more traditional cytogenetic analysis techniques of Giemsa banding and fluorescence in situ hybridization (FISH) which are limited by the resolution of the microscope utilized.

This is achieved through the use of competitive fluorescence in situ hybridization. In short, this involves the isolation of DNA from the two sources to be compared, most commonly a test and reference source, independent labelling of each DNA sample with a different fluorophores (fluorescent molecules) of different colors (usually red and green), denaturation of the DNA so that it is single stranded, and the hybridization of the two resultant samples in a 1:1 ratio to a normal metaphase spread of chromosomes, to which the labelled DNA samples will bind at their locus of origin. Using a fluorescence microscope and computer software, the differentially colored fluorescent signals are then compared along the length of each chromosome for identification of chromosomal differences between the two sources. A higher intensity of the test sample color in a specific region of a chromosome indicates the gain of material of that region in the corresponding source sample, while a higher intensity of the reference sample colour indicates the loss of material in the test sample in that specific region. A neutral color (yellow when the fluorophore labels are red and green) indicates no difference between the two samples in that location.

CGH is only able to detect unbalanced chromosomal abnormalities. This is because balanced chromosomal abnormalities, such as reciprocal translocations, inversions or ring chromosomes, do not affect copy number that is detected by CGH technologies. CGH does, however, allow for the exploration of all 46 human chromosomes in single test and the discovery of deletions and duplications, even on the microscopic scale which may lead to the identification of candidate genes to be further explored by other cytological techniques.

Through the use of DNA microarrays in conjunction with CGH techniques, the more specific form of array CGH (aCGH) has been developed, allowing for a locus-by-locus measure of CNV with increased resolution as low as 100 kilobases. This improved technique allows for the etiology of known and unknown conditions to be discovered.

In making assessments of decreased or increased copy number, one will reference the copy number of genes that do not vary in copy number, such as housekeeping genes including β-actin and GAPDH.

Metaphase Slide Preparation.

The DNA on the slide is a reference sample, and is thus obtained from a karyotypically normal man or woman, though it is preferential to use female DNA as they possess two X chromosomes which contain far more genetic information than the male Y chromosome. Phytohaemagglutinin stimulated peripheral blood lymphocytes are used. 1 mL of heparinised blood is added to 10 ml of culture medium and incubated for 72 hours at 37° C. in an atmosphere of 5% CO₂. Colchicine is added to arrest the cells in mitosis, the cells are then harvested and treated with hypotonic potassium chloride and fixed in 3:1 methanollacetic acid.

One drop of the cell suspension should then be dropped onto an ethanol cleaned slide from a distance of about 30 cm, optimally this should be carried out at room temperature at humidity levels of 60-70%. Slides should be evaluated by visualization using a phase contrast microscope, minimal cytoplasm should be observed and chromosomes should not be overlapping and be 400-550 bands long with no separated chromatids and finally should appear dark rather than shiny. Slides then need to be air dried overnight at room temperature, and any further storage should be in groups of four at −20° C. with either silica beads or nitrogen present to maintain dryness. Different donors should be tested as hybridization may be variable. Commercially available slides may be used, but should always be tested first.

Isolation of DNA from Test Tissue and Reference Tissue.

Standard phenol extraction is used to obtain DNA from test or reference (karyotypically normal individual) tissue, which involves the combination of Tris-ethylenediaminetetraacetic acid and phenol with aqueous DNA in equal amounts. This is followed by separation by agitation and centrifugation, after which the aqueous layer is removed and further treated using ether and finally ethanol precipitation is used to concentrate the DNA. This step may be completed using DNA isolation kits available commercially which are based on affinity columns.

Preferentially, DNA should be extracted from fresh or frozen tissue as this will be of the highest quality, though it is now possible to use archival material which is fomaline fixed or paraffin wax embedded, provided the appropriate procedures are followed. 0.5-1 μg of DNA is sufficient for the CGH experiment, though if the desired amount is not obtained DOP-PCR may be applied to amplify the DNA, however it in this case it is important to apply DOP-PCR to both the test and reference DNA samples to improve reliability.

DNA Labelling.

Nick translation is used to label the DNA and involves cutting DNA and substituting nucleotides labelled with fluorophores (direct labelling) or biotin or oxigenin to have fluophore conjugated antibodies added later (indirect labelling). It is then important to check fragment lengths of both test and reference DNA by gel electrophoresis, as they should be within the range of 500 kb-1500 kb for optimum hybridization.

Blocking.

Unlabelled Life Technologies Corporation's Cot-1 DNA® (placental DNA enriched with repetitive sequences of length 50-100 bp) is added to block normal repetitive DNA sequences, particularly at centromeres and telomeres, as if these sequences are detected, they may reduce the fluorescence ratio and cause gains or losses to escape detection.

Hybridization.

8-12 μl of each of labelled test and labelled reference DNA are mixed and 40 μg Cot-1 DNA® is added, then precipitated and subsequently dissolved in 6 μl of hybridization mix, which contains 50% formamide to decrease DNA melting temperature and 10% dextran sulphate to increase the effective probe concentration in a saline sodium citrate (SSC) solution at a pH of 7.0.

Denaturation of the slide and probes are carried out separately. The slide is submerged in 70% formamide/2×SSC for 5-10 minutes at 72° C., while the probes are denatured by immersion in a water bath of 80° C. for 10 minutes and are immediately added to the metaphase slide preparation. This reaction is then covered with a coverslip and left for two to four days in a humid chamber at 40° C.

The coverslip is then removed and 5 minute washes are applied, three using 2×SSC at room temperature, one at 45° C. with 0.1×SSC and one using TNT at room temperature. The reaction is then preincubated for 10 minutes then followed by a 60 minute, 37° C. incubation, three more 5 minute washes with TNT then one with 2×SSC at room temperature. The slide is then dried using an ethanol series of 70%/96%/100% before counterstaining with DAPI (0.35 μg/ml), for chromosome identification, and sealing with a coverslip.

Fluorescence Visualisation and Imaging.

A fluorescence microscope with the appropriate filters for the DAPI stain as well as the two fluorophores utilised is required for visualisation, and these filters should also minimise the crosstalk between the fluorophores, such as narrow band pass filters. The microscope must provide uniform illumination without chromatic variation, be appropriately aligned and have a “plan” type of objective which is apochromatic and give a magnification of ×63 or ×100.

The image should be recorded using a camera with spatial resolution at least 0.1 μm at the specimen level and give an image of at least 600×600 pixels. The camera must also be able to integrate the image for at least 5 to 10 seconds, with a minimum photometric resolution of 8 bit.

Dedicated CGH software is commercially available for the image processing step, and is required to subtract background noise, remove and segment materials not of chromosomal origin, normalize the fluorescence ratio, carry out interactive karyotyping and chromosome scaling to standard length. A “relative copy number karyotype” which presents chromosomal areas of deletions or amplifications is generated by averaging the ratios of a number of high quality metaphases and plotting them along an ideogram, a diagram identifying chromosomes based on banding patterns. Interpretation of the ratio profiles is conducted either using fixed or statistical thresholds (confidence intervals). When using confidence intervals, gains or losses are identified when 95% of the fluorescence ratio does not contain 1.0.

Extreme care must be taken to avoid contamination of any step involving DNA, especially with the test DNA as contamination of the sample with normal DNA will skew results closer to 1.0, thus abnormalities may go undetected. FISH, PCR and flow cytometry experiments may be employed to confirm results.

Array comparative genomic hybridization (also microarray-based comparative genomic hybridization, matrix CGH, array CGH, aCGH) is a molecular cytogenetic technique for the detection of chromosomal copy number changes on a genome wide and high-resolution scale. Array CGH compares the patient's genome against a reference genome and identifies differences between the two genomes, and hence locates regions of genomic imbalances in the patient, utilizing the same principles of competitive fluorescence in situ hybridization as traditional CGH.

With the introduction of array CGH, the main limitation of conventional CGH, a low resolution, is overcome. In array CGH, the metaphase chromosomes are replaced by cloned DNA fragments (+100-200 kb) of which the exact chromosomal location is known. This allows the detection of aberrations in more detail and, moreover, makes it possible to map the changes directly onto the genomic sequence.

Array CGH has proven to be a specific, sensitive, fast and highthroughput technique, with considerable advantages compared to other methods used for the analysis of DNA copy number changes making it more amenable to diagnostic applications. Using this method, copy number changes at a level of 5-10 kilobases of DNA sequences can be detected. As of 2006, even high-resolution CGH (HR-CGH) arrays are accurate to detect structural variations (SV) at resolution of 200 bp. This method allows one to identify new recurrent chromosome changes such as microdeletions and duplications in human conditions such as cancer and birth defects due to chromosome aberrations.

Array CGH is based on the same principle as conventional CGH. In both techniques, DNA from a reference (or control) sample and DNA from a test (or patient) sample are differentially labelled with two different fluorophores and used as probes that are cohybridized competitively onto nucleic acid targets. In conventional CGH, the target is a reference metaphase spread. In array CGH, these targets can be genomic fragments cloned in a variety of vectors (such as BACs or plasmids), cDNAs, or oligonucleotides.

DNA from the sample to be tested can labeled with a green fluorophore (Cyanine 3) and a reference DNA sample can labeled with red fluorophore (Cyanine 5). Equal quantities of the two DNA samples are mixed and cohybridized to a DNA microarray of several thousand evenly spaced cloned DNA fragments or oligonucleotides, which have been spotted in triplicate on the array. After hybridization, digital imaging systems are used to capture and quantify the relative fluorescence intensities of each of the hybridized fluorophores. The resulting ratio of the fluorescence intensities is proportional to the ratio of the copy numbers of DNA sequences in the test and reference genomes. If the intensities of the flurochromes are equal on one probe, this region of the patient's genome is interpreted as having equal quantity of DNA in the test and reference samples; if there is an altered Cy3:Cy5 ratio this indicates a loss or a gain of the patient DNA at that specific genomic region.

Conventional CGH has been used mainly for the identification of chromosomal regions that are recurrently lost or gained in tumors, as well as for the diagnosis and prognosis of cancer. This approach can also be used to study chromosomal aberrations in fetal and neonatal genomes. Furthermore, conventional CGH can be used in detecting chromosomal abnormalities and have been shown to be efficient in diagnosing complex abnormalities associated with human genetic disorders.

CGH data from several studies of the same tumor type show consistent patterns of non-random genetic aberrations. Some of these changes appear to be common to various kinds of malignant tumors, while others are more tumor specific. For example, gains of chromosomal regions 1q, 3q and 8q, as well as losses of 8p, 13q, 16q and 17p, are common to a number of tumor types, such as breast, ovarian, prostate, renal and bladder cancer. Other alterations, such as 12p and Xp gains in testicular cancer, 13q gain 9q loss in bladder cancer, 14q loss in renal cancer and Xp loss in ovarian cancer are more specific, and might reflect the unique selection forces operating during cancer development in different organs.

Array CGH applications are mainly directed at detecting genomic abnormalities in cancer. However, array CGH is also suitable for the analysis of DNA copy number aberrations that cause human genetic disorders. That is, array CGH is employed to uncover deletions, amplifications, breakpoints and ploidy abnormalities. Earlier diagnosis is of benefit to the patient as they may undergo appropriate treatments and counseling to improve their prognosis.

Genetic alterations and rearrangements occur frequently in cancer and contribute to its pathogenesis. Detecting these aberrations by array CGH provides information on the locations of important cancer genes and can have clinical use in diagnosis, cancer classification and prognostication. However, not all of the losses of genetic material are pathogenetic, since some DNA material is physiologically lost during the rearrangement of immunoglobulin subgenes.

Array CGH may also be applied not only to the discovery of chromosomal abnormalities in cancer, but also to the monitoring of the progression of tumors. Differentiation between metastatic and mild lesions is also possible using FISH once the abnormalities have been identified by array CGH.

3. Northern Blot

The northern blot is a technique used in molecular biology research to study gene expression by detection of RNA (or isolated mRNA) in a sample. With northern blotting it is possible to observe cellular control over structure and function by determining the particular gene expression levels during differentiation, morphogenesis, as well as abnormal or diseased conditions. Northern blotting involves the use of electrophoresis to separate RNA samples by size and detection with a hybridization probe complementary to part of or the entire target sequence. The term ‘northern blot’ actually refers specifically to the capillary transfer of RNA from the electrophoresis gel to the blotting membrane. However, the entire process is commonly referred to as northern blotting.

A general blotting procedure starts with extraction of total RNA from a homogenized tissue sample or from cells. Eukaryotic mRNA can then be isolated through the use of oligo (dT) cellulose chromatography to isolate only those RNAs with a poly(A) tail. RNA samples are then separated by gel electrophoresis. Since the gels are fragile and the probes are unable to enter the matrix, the RNA samples, now separated by size, are transferred to a nylon membrane through a capillary or vacuum blotting system.

A nylon membrane with a positive charge is the most effective for use in northern blotting since the negatively charged nucleic acids have a high affinity for them. The transfer buffer used for the blotting usually contains formamide because it lowers the annealing temperature of the probe-RNA interaction, thus eliminating the need for high temperatures, which could cause RNA degradation. Once the RNA has been transferred to the membrane, it is immobilized through covalent linkage to the membrane by UV light or heat. After a probe has been labeled, it is hybridized to the RNA on the membrane. Experimental conditions that can affect the efficiency and specificity of hybridization include ionic strength, viscosity, duplex length, mismatched base pairs, and base composition. The membrane is washed to ensure that the probe has bound specifically and to prevent background signals from arising. The hybrid signals are then detected by X-ray film and can be quantified by densitometry. To create controls for comparison in a northern blot samples not displaying the gene product of interest can be used after determination by microarrays or RT-PCR.

The RNA samples are most commonly separated on agarose gels containing formaldehyde as a denaturing agent for the RNA to limit secondary structure. The gels can be stained with ethidium bromide (EtBr) and viewed under UV light to observe the quality and quantity of RNA before blotting. Polyacrylamide gel electrophoeresis with urea can also be used in RNA separation but it is most commonly used for fragmented RNA or microRNAs. An RNA ladder is often run alongside the samples on an electrophoresis gel to observe the size of fragments obtained but in total RNA samples the ribosomal subunits can act as size markers. Since the large ribosomal subunit is 28S (approximately 5 kb) and the small ribosomal subunit is 18S (approximately 2 kB) two prominent bands appear on the gel, the larger at close to twice the intensity of the smaller.

Probes for northern blotting are composed of nucleic acids with a complementary sequence to all or part of the RNA of interest, they can be DNA, RNA, or oligonucleotides with a minimum of 25 complementary bases to the target sequence. RNA probes (riboprobes) that are transcribed in vitro are able to withstand more rigorous washing steps preventing some of the background noise. Commonly cDNA is created with labelled primers for the RNA sequence of interest to act as the probe in the northern blot. The probes must be labelled either with radioactive isotopes (³²P) or with chemiluminescence in which alkaline phosphatase or horseradish peroxidase break down chemiluminescent substrates producing a detectable emission of light. The chemiluminescent labelling can occur in two ways: either the probe is attached to the enzyme, or the probe is labelled with a ligand (e.g., biotin) for which the antibody (e.g., avidin or streptavidin) is attached to the enzyme. X-ray film can detect both the radioactive and chemiluminescent signals and many researchers prefer the chemiluminescent signals because they are faster, more sensitive, and reduce the health hazards that go along with radioactive labels. The same membrane can be probed up to five times without a significant loss of the target RNA.

4. Fluorescence In Situ Hybridization and Chromogenic In Situ Hybridization

Fluorescence in situ hybridization (FISH) can be used for molecular studies. FISH is used to detect highly specific DNA probes which have been hybridized to chromosomes using fluorescence microscopy. The DNA probe is labeled with fluorescent or non fluorescent molecules which are then detected by fluorescent antibodies. The probes bind to a specific region or regions on the target chromosome. The chromosomes are then stained using a contrasting color, and the cells are viewed using a fluorescence microscope.

Each FISH probe is specific to one region of a chromosome, and is labeled with fluorescent molecules throughout its length. Each microscope slide contains many metaphases. Each metaphase consists of the complete set of chromosomes, one small segment of which each probe will seek out and bind itself to. The metaphase spread is useful to visualize specific chromosomes and the exact region to which the probe binds. The first step is to break apart (denature) the double strands of DNA in both the probe DNA and the chromosome DNA so they can bind to each other. This is done by heating the DNA in a solution of formamide at a high temperature (70-75° C.). Next, the probe is placed on the slide and the slide is placed in a 37° C. incubator overnight for the probe to hybridize with the target chromosome. Overnight, the probe DNA seeks out its target sequence on the specific chromosome and binds to it. The strands then slowly reanneal. The slide is washed in a salt/detergent solution to remove any of the probe that did not bind to chromosomes and differently colored fluorescent dye is added to the slide to stain all of the chromosomes so that they may then be viewed using a fluorescent light microscope. Two, or more different probes labeled with different fluorescent tags can be mixed and used at the same time. The chromosomes are then stained with a third color for contrast. This gives a metaphase or interphase cell with three or more colors which can be used to detect different chromosomes at the same time, or to provide a control probe in case one of the other target sequences are deleted and a probe cannot bind to the chromosome. This technique allows, for example, the localization of genes and also the direct morphological detection of genetic defects.

The advantage of using FISH probes over microsatellite instability to test for copy is to that the:
(a) FISH is easily and rapidly performed on cells of interest and can be used on paraffin-embedded, or fresh or frozen tissue allowing the use of micro-dissection;
(b) specific gene changes can be analyzed on a cell by cell basis in relationship to centomeric probes so that true homozygosity versus heterozygosity of a DNA sequence can be evaluated (use of PCR™ for microsatellite instability may permit amplification of surrounding normal DNA sequences from contamination by normal cells in a homozygously deleted region imparting a false positive impression that the allele of interest is not deleted);
(c) PCR cannot identify amplification of genes; and
(d) FISH using bacterial artificial chromosomes (BACs) permits easy detection and localization on specific chromosomes of genes of interest which have been isolated using specific primer pairs.
   Chromogenic in situ hybridzation (CISH) enables the gain genetic information in the context of tissue morphology using methods already present in histology labs. CISH allows detection of gene amplification, chromosome translocations and chromosome number using conventional enzymatic reactions under the brightfield microscope on formalin-fixed, paraffin-embedded (FFPE) tissues. U.S. Publication No. 2009/0137412, incorporated herein by reference. The scanning may be performed, for example, on an automated scanner with Fluorescence capabilities (Bioview System, Rehovot, Israel).

### IV. TREATMENT OF CANCER

In accordance with the present disclosure, there are provided methods of treatment for cancers, particularly those where Kub5/Hera is over- or under-expressed, or is mutated in certain functional domains. Because Kub5/Hera has been demonstrated to be involved in the repair of double-stranded breaks (DSBs), its overexpression will result in diminished activity of agents that act through the generate of DSBs. Such patients should be guided towards different therapies. However, where Kub5/Hera levels are lower than normal cells, agents generating DSBs should have particular value, including combinations of

A. Formulation and Administration

The present disclosure provides pharmaceutical compositions. Such compositions comprise a prophylactically or therapeutically effective amount of an agent, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intra-arterial, intrabuccal, intranasal, nebulized, bronchial inhalation, or delivered by mechanical ventilation.

Pharmaceutical compositions of the present disclosure, as described herein, can be formulated for parenteral administration, e.g., formulated for injection via the intradermal, intravenous, intramuscular, subcutaneous, intra-tumoral or even intraperitoneal routes. The antibodies could alternatively be administered by a topical route directly to the mucosa, for example by nasal drops, inhalation, or by nebulizer. Pharmaceutically acceptable salts, include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

B. Mono- and Combination Therapies

As discussed above, the present disclosure is designed to guide therapeutic decision making. As such, the methods may involve various monotherapies, or may involve the combination of two or more therapeutic regimens, depending on the Kub5/Hera genotype/phenotype of the patient. These therapies would be provided individually or in a combined amount effective to achieve a reduction in one or more disease parameters. In the case of combination therapies, this process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the primary therapy and the other includes the other agent.

Alternatively, the primary therapy may precede or follow the other treatment by intervals ranging from minutes to weeks. One would generally ensure that a significant period of time did not expire between the time of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 12 hours. In some situations, it may be desirable to extend the time period for treatment significantly; however, where several 10 days (2, 3, 4, 5, 6 or 7) to several weeks (1, 2, 3, 4, 5, 6, 7 or 8) lapse between the respective administrations.

It also is conceivable that more than one administration of either the primary therapy or the other therapy will be desired. Various combinations may be employed, where the primary therapy is “A,” and the other therapy is “B,” as exemplified below:

A/B/A B/A/B B/B/A A/A/B B/A/A A/B/B B/B/B/A
B/B/A/B A/A/B/B A/B/A/B A/B/B/A B/B/A/A B/A/B/A
B/A/A/B B/B/B/A A/A/A/B B/A/A/A A/B/A/A A/A/B/A
A/B/B/B B/A/B/B B/B/A/B

Other combinations are contemplated. To kill cells, inhibit cell growth, inhibit metastasis, inhibit angiogenesis or otherwise reverse or reduce the malignant phenotype of tumor cells, using the methods and compositions of the present invention, one may contact a target cell or site with an primary therapy and at least one other therapy. These therapies would be provided in a combined amount effective to kill or inhibit proliferation of cancer cells. This process may involve contacting the cells/site/subject with the agents/therapies at the same time.

1. Chemotherapy

The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ1 and calicheamicin ω1; dynemicin, including dynemicin A uncialamycin and derivatives thereof; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and docetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

PARP inhibitors are of particular relevance to the present disclosure. The compounds are a group of pharmacological inhibitors of the enzyme poly ADP ribose polymerase 1 (PARP1). They are developed for multiple indications; the most important is the treatment of cancer. Several forms of cancer are more dependent on PARP than regular cells, making PARP an attractive target for cancer therapy. In addition to their use in cancer therapy, PARP1 inhibitors (PARP1i) are considered a potential treatment for acute life-threatening diseases, such as stroke and myocardial infarction, as well as for long-term neurodegenerative diseases. DNA is damaged thousands of times during each cell cycle, and that damage must be repaired.

BRCA1, BRCA2 and PALB2 are proteins that are important for the repair of double-strand DNA breaks by the error-free homologous recombinational repair, or HRR, pathway. When the gene for either protein is mutated, the change can lead to errors in DNA repair that can eventually cause breast cancer. When subjected to enough damage at one time, the altered gene can cause the death of the cells.

PARP1 is a protein that is important for repairing single-strand breaks (‘nicks’ in the DNA). If such nicks persist unrepaired until DNA is replicated (which must precede cell division), then the replication itself can cause double strand breaks to form.

Drugs that inhibit PARP1 cause multiple double strand breaks to form in this way, and in tumours with BRCA1, BRCA2 or PALB2 mutations these double strand breaks cannot be efficiently repaired, leading to the death of the cells. Normal cells that don't replicate their DNA as often as cancer cells, and that lack any mutated BRCA1 or BRCA2 still have homologous repair operating, which allows them to survive the inhibition of PARP.

Some cancer cells that lack the tumor suppressor PTEN may be sensitive to PARP inhibitors because of downregulation of Rad51, a critical homologous recombination component, although other data suggest PTEN may not regulate Rad51. Hence PARP inhibitors may be effective against many PTEN-defective tumours (e.g., some aggressive prostate cancers). Cancer cells that are low in oxygen (e.g., in fast growing tumors) are sensitive to PARP inhibitors.

Previously, PARP inhibitors were thought to work primarily by blocking PARP enzyme activity, thus preventing the repair of DNA damage and ultimately causing cell death. More recently, however, scientists have established that PARP inhibitors have an additional mode of action: localizing PARP proteins at sites of DNA damage, which has relevance to their anti-tumor activity. The trapped PARP protein-DNA complexes are highly toxic to cells because they block DNA replication. When the researchers tested three PARP inhibitors for their differential ability to trap PARP proteins on damaged DNA, they found that the trapping potency of the inhibitors varied widely. The PARP family of proteins in humans includes PARP1 and PARP2, which are DNA binding and repair proteins. When activated by DNA damage, these proteins recruit other proteins that do the actual work of repairing DNA. Under normal conditions, PARP1 and PARP2 are released from DNA once the repair process is underway. However, as this study shows, when they are bound to PARP inhibitors, PARP1 and PARP2 become trapped on DNA. The researchers showed that trapped PARP-DNA complexes are more toxic to cells than the unrepaired single-strand DNA breaks that accumulate in the absence of PARP activity, indicating that PARP inhibitors act as PARP poisons. These findings suggest that there may be two classes of PARP inhibitors, catalytic inhibitors that act mainly to inhibit PARP enzyme activity and do not trap PARP proteins on DNA, and dual inhibitors that both block PARP enzyme activity and act as PARP poison.

The following PARP inhibitors have started Phase III clincial trials:
Iniparib (BSI 201) for breast cancer and squamous cell lung cancer (failed trial for triple negative breast cancer);
BMN-673 after trials for advanced hematological malignancies and for advanced or recurrent solid tumors; now in phase 3 for metastatic germline BRCA mutated breast cancer.
   The following drugs have Started Phase II clinical trials:
Olaparib (AZD-2281) for breast, ovarian and colorectal cancer; progressing to phase III;
Rucaparib (AG014699, PF-01367338) for metastatic breast and ovarian cancer;
Veliparib (ABT-888) for metastatic melanoma and breast cancer
CEP 9722 for non-small-cell lung cancer (NSCLC)
   The following drugs have Started Phase I clinical trials:
MK 4827 Inhibitor of PARP1 and PARP2;
BGB-290 1st dosing July 2014
3-aminobenzamide, a prototypical PARP inhibitor, is in the experimental, pre-clinical phase.

PARP inhibitors may advantageously be used in combination with radiation. The main function of radiotherapy is to produce DNA strand breaks, causing severe DNA damage and leading to cell death. Radiotherapy has the potential to kill 100% of any targeted cells, but the dose required to do so would cause unacceptable side effects to healthy tissue. Radiotherapy therefore can only be given up to a certain level of radiation exposure. Combining radiation therapy with PARP inhibitors therefore offers promise, since the inhibitors would lead to formation of double strand breaks from the single-strand breaks generated by the radiotherapy in tumor tissue with BRCA1/BRCA2 mutations. This combination could therefore lead to either more powerful therapy with the same radiation dose or similarly powerful therapy with a lower radiation dose.

2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present invention may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wks), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and may be used as an alternative to the metal blocks. The multi-leaf collimator consists of a number of metal sheets, which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing to radiation. Radiosensitizers make tumor cells more likely to be damaged, and radioprotectors protect normal tissues from the effects of radiation. Hyperthermia, the use of heat, is also being studied for its effectiveness in sensitizing tissue to radiation.

3. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

In some particular embodiments, after removal of the tumor, an adjuvant treatment with a compound of the present disclosure is believe to be particularly efficacious in reducing the reoccurance of the tumor. Additionally, the compounds of the present disclosure can also be used in a neoadjuvant setting.

4. Other Agents

It is contemplated that other agents may be used with the present invention. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1β, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents may be used in combination with the present invention to improve the anti-hyerproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

It also should be pointed out that any of the foregoing therapies may prove useful by themselves in treating cancer.

### V. RISK OF DNA DAMAGE

DNA damage is known to play a major role in mutagenesis and carcinogenesis. The chemical events that lead to DNA damage include hydrolysis, exposure to reactive oxygen substances (ROS) and other reactive metabolites. These reactions are triggered by exposure to exogenous chemicals, low dose radiation or they can result from metabolic, endogenous processes. These threats are even greater when endogenous DNA repair mechanisms are compromised. Indeed, the concentrations and mutagenic potentials of known environmental carcinogens are insufficient to explain the high incidence of sporadic cancer that is observed.

Possible sources of DNA damage that pose an increased risk to subjects with mutations in KUB5/HERA include radiations, such as from industrial sources, diagnostic x-rays or therapeutic x-rays (such as targeted radiation in cancer therapy, or ablative radiation prior to bone marrow transplant), or environmental carcinogenic chemicals, such as those used industrial processes, agriculture (herbicides, insecticides, etc.). DNA damage may also be due to chronic inflammatory states such as gastro-esophageal reflux disorder, esophagitis, gastritis, colitis, colonitis, or pancreatitis.

### VI. EXAMPLES

The following examples are included to demonstrate preferred embodiments. It should be appreciated by those of skill in the art that the techniques disclosed in the examples that follow represent techniques discovered by the inventors to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Cell Lines and Reagents.

Breast cancer cell lines were obtained from the American Type Culture Collection (Manassas, Va.) and from the cell repository of the Hamon Center for Therapeutic Oncology Research at UT Southwestern Medical Center. MDA-MB-231 cells expressing shRNA for K-H or PARP1 were generated and maintained in RPMI media supplemented with 10% fetal bovine serum (FBS) at 37° C. in a humidified CO₂ (5%) incubator. AG014361, AG014699 (Rucaparib) and RO3066 (CDK1 inhibitor) were purchased from Selleck Chemicals at the highest purity.

Transfection.

RNA oligonucleotide pairs specific for endogenous RPRD1B, CDK1. PARP1, and RAD51 were obtained pre-annealed from commercial sources (Sigma and Dharmacon) and delivered to cells by Oligofectamine (Invitrogen) according to the manufacturer's instructions (Lipofectamine 2000 for cDNA and/or si-RNA transfection; RNAiMax for siRNA only transfection). Oligonucleotide sequences used in this study are summarized in Supplementary Table S4. Briefly, cells were plated at log phase in 6- or 10-cm diameter wells in antibiotic-free medium the day before transfection. The following day, a mixture containing siRNA (e.g., in a 10 cm dish with final volume of 5 mL: 5 μL of 15 μM stock siRNA solution and Lipofectamine 2000 (10 μL) in Opti-MEM (1 mL total) was prepared according to the manufacturer's instructions and added to cells with 4 mL of antibiotic-free medium dropwise, to give a final siRNA concentration of ˜15 nM.

PARP Activity Assay.

PARP activity was measured using immunoblot assay, which measured the amount of poly(ADP)-ribonucleotide (PAR) products synthesized using total protein lysates for PARP enzymatic activity in vitro. Briefly, the reaction containing total protein lysate (90 μg), and excess blunt-ended double-strand DNA oligonucleotide (26-mer) with internal synthetic abasic site (50 uM) in a reaction buffer containing Tris-HCl (10 mM, pH=7.4) and Mg²⁺ (12 mM) was initiated by addition of excess β-NAD⁺ (10 mM). The reaction was then stopped at various time-points by the addition of ice-cold concentrated AG014699/Rucaparib (250 μM), a known PARP1 inhibitor. Aliquots from each time points of the reaction were blotted onto a nitrocellulose membrane (BioRad dot blotter), and processed using standard Western blot assays. The density of each dots were quantified by ImageJ and plotted by GraphPad PRISM after normalization to controls (no NAD⁺ addition).

Western Blot Analysis.

Whole-cell lysates and nuclear extracts were prepared as previously described (Morales et al., 2014). See Supplementary Table S2 for a list of specific antibodies and dilutions used. Protein concentrations were determined by BCA Assay following manufacturer's instructions (Thermo Scientific). Normalized protein samples were separated on denaturing gradient gels (4-20% from BioRad) and were transferred to PVDF membranes for immunoblotting. After transfer, the membranes were first blocked with 1× Sigma Blocking Buffer for an hour at room temperature. The membranes were then incubated with the indicated primary antibodies diluted in 1× Sigma Blocking Buffer at 4° C. overnight. After this step, the membranes were washed with PBST (3×, 10 minutes each). HRP-conjugated secondary antibodies were diluted in 1× Sigma Blocking Buffer and incubated with the membranes at room temperature for 1 hr. After this incubation period, the membranes were washed with PBST (3×, 15 minutes each) and processed for chemiluminescence signal using the SuperSignal West Pico Substrate (Thermo Scientific) or Clarity Western ECL Substrate (BioRad).

Immunofluorescence (IF).

MDA-MB-231 shSCR or shK-H cells were plated in a six-well plate (˜200,000 each well containing a glass slide) overnight. The next day, cells were treated with DMSO (0.01%) or Rucaparib/AG014699 (5 μM) for 24 hr. After treatment, the media was gently replaced with drug-free media and harvested at various times (0, 1, 6, 24 hr). The slides were gently washed with phosphate-buffered solution (pH=7.4), and fixed with ice cold methanol:acetone solution (3:1) for 10 min at −20° C. The cells were then washed and rehydrated with PBS at room temperature (3×, 5 min intervals). Next, the cells were incubated in blocking solution (5% normal goat serum in PBS) for 60 min at RT. After blocking, the cells were then incubated in primary antibody (diluted in 5% normal goat serum in PBS) overnight at 4° C. The next day, the cells were washed three times with wash buffer (1% BSA in PBS; 5 min. interval between washes). The cells were incubated in fluorescent secondary antibody diluted in 1% BSA/2.5% normal goat serum/PBS solution for 1 hr at RT with minimal exposure to light. The cells were then washed three times with wash buffer (1% BSA in PBS; 5 min. intervals). Finally, the last wash buffer was completely aspirated, and the cover glass was mounted with Vectashield mounting medium with DAPI. Images were acquired using DM5500 Upright microscope (Leica) equipped with a digital camera.

Colony Formation Assay (CFA).

Cells were seeded out in appropriate dilutions and treated with the vehicle (0.01% DMSO) or PARP inhibitors at various concentrations for 24 hr. After treatment, cells were then cultured in drug-free medium for several days (at least six rounds of replication), depending on the proliferation rate of each cell line. Colonies were fixed with formaldehyde (4%) and stained with crystal violet. Colonies containing more than 50 normal-appearing cells were counted using a microscope. Each drug concentration was assessed in triplicate, and the experiments were repeated three times.

TUNEL Assay.

To measure apoptotic cell death, log phase 231 shSCR or shK-H cells were treated with the vehicle (0.01% DMSO) or Rucaparib (5 and 10 μM) for 24 hr, and incubated in drug-free media for additional 72 hr in the cell incubator. The cells were then harvested and fixed in 70% ethanol overnight at −20° C. Equal amount of cells from each treatment were then subjected to terminal deoxynucleotidyltransferase dUTP nick end labeling (TUNEL) following the standard protocols from APO-Direct TUNEL Assay kit (BD Biosciences), which include positive and negative controls. Flow cytometry experiments were immediately performed using the BD LSRFortessa II cell analyzer, and data were analyzed using the BD FACSDiva software (www.bdbiosciences.com). All experiments were performed in triplicate.

HR Activity Assay.

To measure HR activity, GFP expression in HEK293 or MCF-7 cells with an integrated DR-GFP reporter was quantified by flow cytometry (36, 58). Briefly, log phase cells were transfected with siRNA (15 nM) against endogenous RAD51, CDK1, K-H or non-targeting control (SCR) using RNAiMax. After 24 hr, cells were co-transfected with specific siRNA as above with the addition of (in siK-H, with or without the myc-K-H or CDK1 expression plasmid (1 μg)) and I-SceI plasmid (1 μg) using Lipofectamine 2000. GFP expression was quantified by flow cytometry after an additional 72 hr using BD LSRFortessa II Cell Analyzer. HR activity was normalized to siSCR treated control.

Array-Based Comparative Genomic Hybridization (aCGH) and Analysis.

Arrays for K-H/RPRD1B copy number alterations (CNA) in each breast cancer cell lines were obtained and analyzed as previously described (Kao et al., 2009). CGH values for K-H gain or loss were obtained via bioinformatics using previously reported dataset (Kao et al., 2009).

Gene Expression Analysis.

For RT-QPCR analysis of gene expression, total cellular RNA was collected using the RNAeasy kit (Qiagen) according to the manufacturer's instructions. RNA quality and concentration were determined using a nanodrop reader. Total RNA (2 μg) was then converted to cDNA using the high-capacity cDNA reverse transcription kit (Applied Biosystems) following the manufacturer's instructions. Gene expression analysis was performed on the ABI-HT7900 (Applied Biosystems) real-time QPCR instrument. Multiplex reactions were performed using TaqMan Gene Expression Master Mix (Applied Biosystems) containing either RPRD1B (Hs01082395_m1), RPRD1B (Hs00221889_m1), or CDK1 (Hs00938777_m1) FAM-labeled TaqMan probes (Applied Biosystems) and the endogenous, normalization control VIC-GAPDH probe (Applied Biosystems; 4326317E-1006036). Triplicate results from multiple experiments were analyzed using the SDS 2.3 software (Applied Biosystems). All samples were normalized to GAPDH expression levels.

Co-Immunoprecipitation (Co-IP).

Log phase cells were plated in 10-cm dishes, and incubated after transfection with either empty pCMV-myc vector, myc-tagged wild-type K-H or myc-tagged mutant K-H (R106A) constructs for 48 hr using Lipofectamine 2000. After transfection, the cells were then harvested and the lysates (1 mg) from each experiment were first pre-cleared with Protein A/G beads (Santa Cruz) prior to overnight incubation at 4° C. with 2 μg of anti-c-myc primary antibody or IgG control conjugated to Protein A/G beads. After incubation, the beads in each experiment were gently washed with TNEN solution [20 mM Tris-HCL (pH 8.0), 0.1 M NaCl, 1 mM EDTA, 0.05% NP-40] (3×, 5 min each using a rotator). The beads were washed one last time with PBS, and boiled in 1× protein loading buffer (5 min). Co-immunoprecipitated proteins (e.g., RNAPII) were analyzed by Western blot as previously described.

Chromatin Immunoprecipitation (ChIP).

Chromatin extracts were prepared as previously described (Wu et al., 2006). Immunoprecipitations were performed with antibodies directed against RNAPII (N-20) or IgG control using standard protocols (ChIP Assay kit and from Millipore) in triplicate. qPCR analysis was performed in triplicate for each replicate sample, and values were normalized to each input and expressed relative to IgG control. Primer sequences for this experiment are listed in Supplementary Table S5.

Luciferase Reporter Assay.

Luciferase activities were assayed with the Dual-Glo® Luciferase Assay System (Promega) with slight modifications from the manufacturer's experimental protocol using the Perkin Elmer Victor X3 Multimode Plate Reader. Firefly luciferase activity was normalized to Renilla luciferase activity as an internal control to compensate for variability in transfection efficiencies. Plasmid pWTCDK1-LUC containing 3 kb of either wild-type or mutant human CDK1 gene promoter upstream of a luciferase reporter gene were prepared as previously described (Badie et al., 2000).

Cells were plated into each well of a 6-well plate at log phase prior to transfection. Empty vector, K-H wildtype or mutant (R106A) cDNA (1 μg) plasmids were first mixed and incubated with pWTCDK1-Luc or a mutated derivative (1 μg) and Renilla Luciferase plasmid (5 ng) in ˜250 μL serum-free OPTI-MEM (TUBE A). In a separate tube containing ˜250 μL serum-free OPTI-MEM, Lipofectamine 2000 (5 μL) was added (TUBE B). After brief incubation, the contents of TUBE A was carefully mixed in with TUBE B and incubated at room temperature for 30 minutes. After this incubation, the solution was gently added dropwise into a well of cells in ˜1 mL of media. The cells were then placed in the incubator for 48 hours.

After incubation, cells were trypsinized, harvested and washed with PBS (2×). The pellets were then lysed in Passive Lysis Buffer (Promega) (100 μL). To assay firefly luciferase activity (expression driven by the CDK1 promoter), luminosity was measured after μL of lysate was mixed with 100 μL of the Dual-Glo® Luciferase Assay Reagent in a 96-well plate. To measure Renilla luciferase activity, luminosity was measured again after addition of 100 μL of the Dual-Glo® Stop & Glo® Reagent. The activities of the experimental reporter (firefly luciferase) were normalized to the activities of the internal control reporter (Renilla luciferase). Reported promoter activities were relative to the siSCR and/or empty vector-treated control. Each assay was assessed in triplicate, and the experiments were repeated three times in two different breast cancer cell lines.

Statistical Analyses.

Statistical analyses were conducted using the GraphPad PRISM Program and in consultation with UT Southwestern's Biostatistics Core. A two-sided student t test was used when appropriate. The results are expressed as the mean±standard error of mean (SEM). A p-value less than 0.05 was considered to be statistically significant.

Proliferation Assay and EdU Click Chemistry.

Asynchronous 231 shSCR versus shK-H cells were plated (40%) and grown in log-phase prior to treatment with EdU (10 μM) for 1 hr at 37° C. in a humidified incubator (5% CO₂). After EdU treatment, the cells were trypsinized and gently washed with PBS (pH=7.4) prior to fixation with ice cold 70% ethanol at −20° C. overnight. After fixation, the cells were washed three times and rehydrated in 1% BSA/PBS at room temperature (5 min. intervals). The cells were then permeabilized with saponin/l % BSA/PBS solution for 1 hr at 37° C. in a humidified incubator. After this step, the cells were washed with 1% BSA/PBS once, and incubated with the ““click” reaction mixture prepared as previously described⁵ 6 but using Alexa-Fluor 647-azide as the conjugating azide dye (3 hr). After incubation, the cells were washed twice with saponin/l % BSA/PBS (5 min. intervals). The cell pellets were then carefully dislodged in PI (10 μg/mL) diluted with saponin/1% BSA/PBS containing RNAse A (100 μg/mL) and incubated for 15 min at 37° C. in a humidified incubator prior to flow cytometer analysis. Flow cytometry experiments were immediately performed using the BD LSRFortessa II cell analyzer, and data were analyzed using the BD FACSDiva software (world-wide-web at bdbiosciences.com).

Molecular Modeling.

Homology modeling studies for K-H were prepared based on previously crystallized structure of RTT103 bound to pS2-CTD heptapeptide fragment of RNAPII CTD tail⁷ to determine crucial K-H•RNAPII molecular interactions. Briefly, K-H and RTI103 primary sequences were aligned, and subjected for homology alignment using the coordinates of RTI103 to build K-H putative structure. These studies were performed to prior to the recent release of K-H RPR domain crystal structure. Molecular Operating Environment (MOE, world-wide-web at chemcomp.com) and PyMol programs were utilized for modeling, simulation, and visualization of structures.

Luciferase Reporter Assay.

Cells were plated into each well of a 6-well plate at log phase prior to transfection. For the knockdown experiments, siSCR or siK-H (15 nM) were first incubated in ˜250 μL serum-free OPTI-MEM (TUBE A). In a separate tube containing ˜250 μL serum-free OPTI-MEM, RNAiMax (5 μL) was added (TUBE B). After brief incubation, the content of TUBE A was carefully mixed in with TUBE B and incubated at room temperature for 30 minutes. After this incubation, the solution was gently added dropwise into each well of cells in ˜1 mL of media. The cells were then placed in the incubator for 24 hr. After 24 hr, the media was aspirated and replaced with a fresh media for a second round of transfection. This time, siSCR or siK-H (15 nM) were first mixed and incubated with pWTCDK1-Luc or a mutated derivative (1 μg) and Renilla Luciferase plasmid (5 ng) in ˜250 μL serum-free OPTI-MEM (TUBE A). In a separate tube containing ˜250 μL serum-free OPTI-MEM, Lipofectamine 2000 (5 μL) was added (TUBE B). After brief incubation, the contents of TUBE A was carefully mixed in with TUBE B and incubated at room temperature for 30 minutes. After this incubation, the solution was gently added dropwise into a well of cells in ˜1 mL of media. The cells were then placed in the incubator for 48 hours.

After incubation, cells were trypsinized, harvested and washed with PBS (2×). The pellets were then lysed in Passive Lysis Buffer (Promega) (100 μL). To assay firefly luciferase activity (expression driven by the CDK1 promoter), luminosity was measured after 20 μL of lysate was mixed with 100 μL of the Dual-Glo® Luciferase Assay Reagent in a 96-well plate. To measure Renilla luciferase activity, luminosity was measured again after addition of 100 μL of the Dual-Glo® Stop & Glo® Reagent. The activities of the experimental reporter (firefly luciferase) were normalized to the activities of the internal control reporter (Renilla luciferase). Reported promoter activities were relative to the siSCR and/or empty vector-treated control. Each assay was assessed in triplicate, and the experiments were repeated three times.

### Example 2—Results

K-H Loss Heightens PARP1 Activity and Potentiates Lethality of PARP1 Inhibitors.

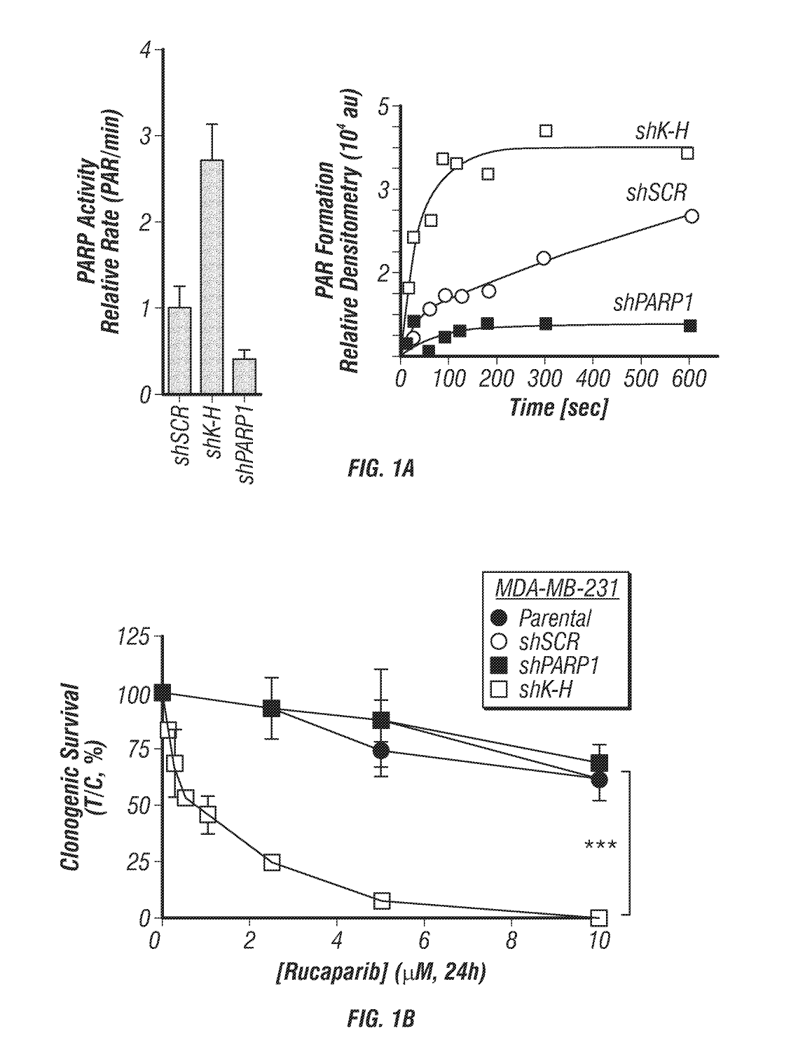

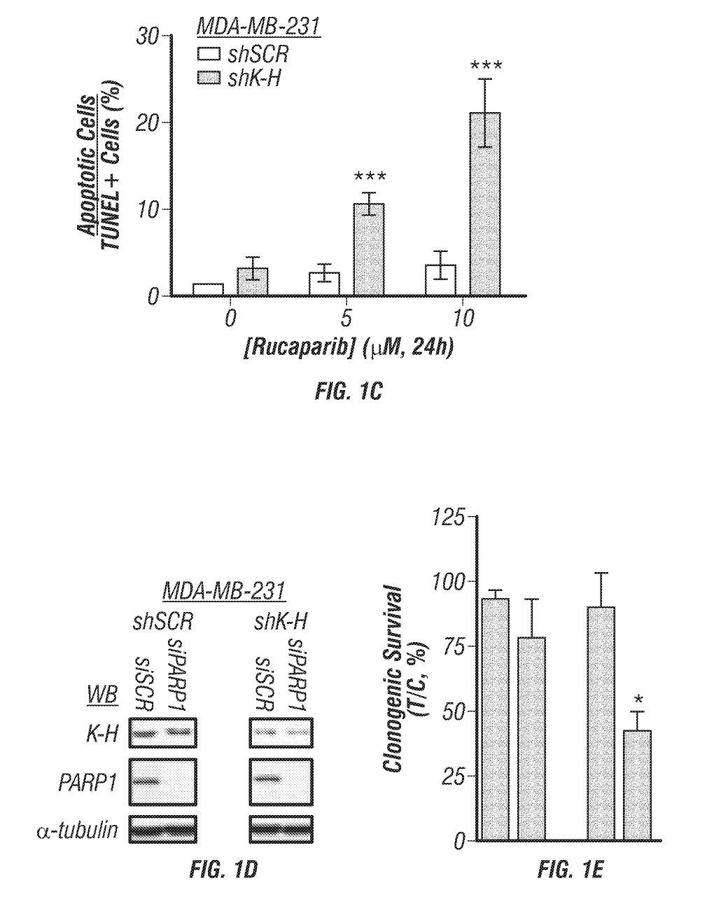

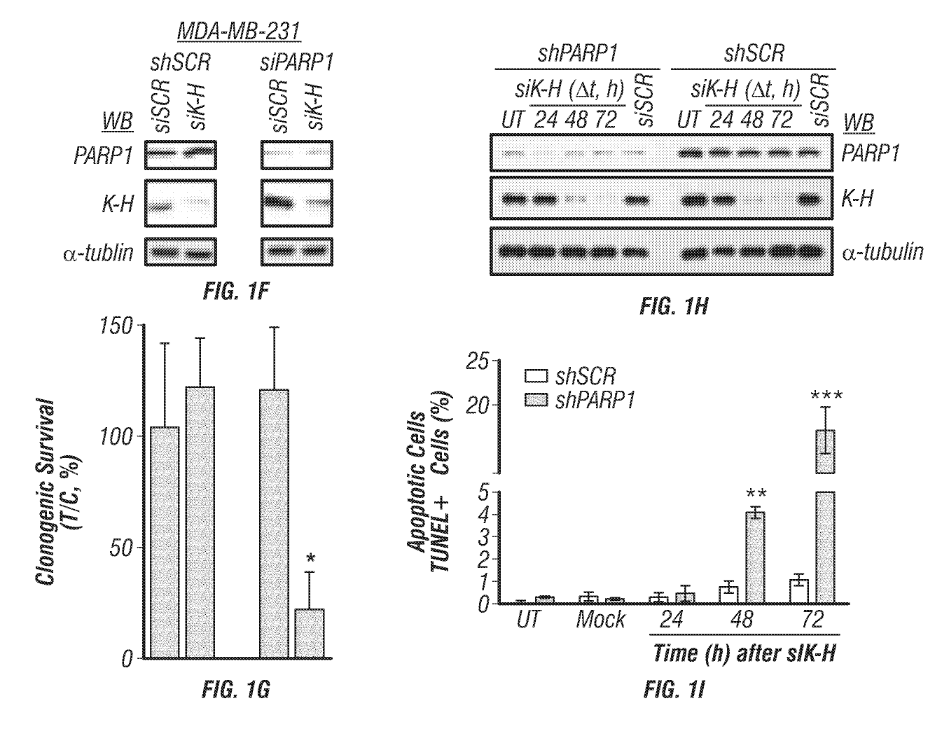

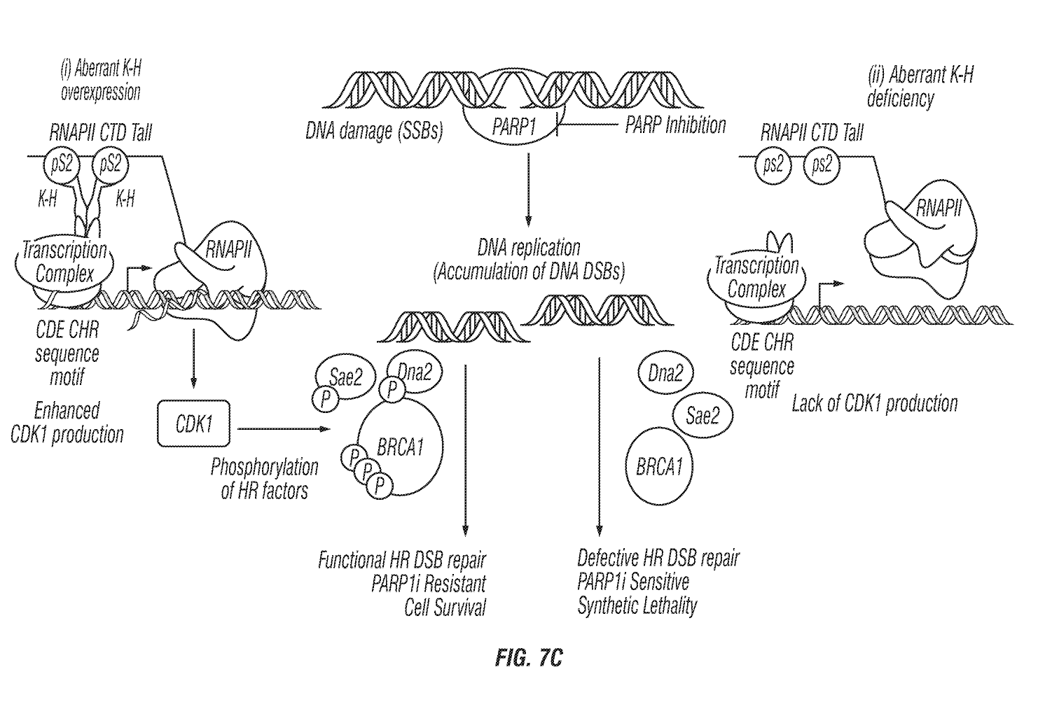

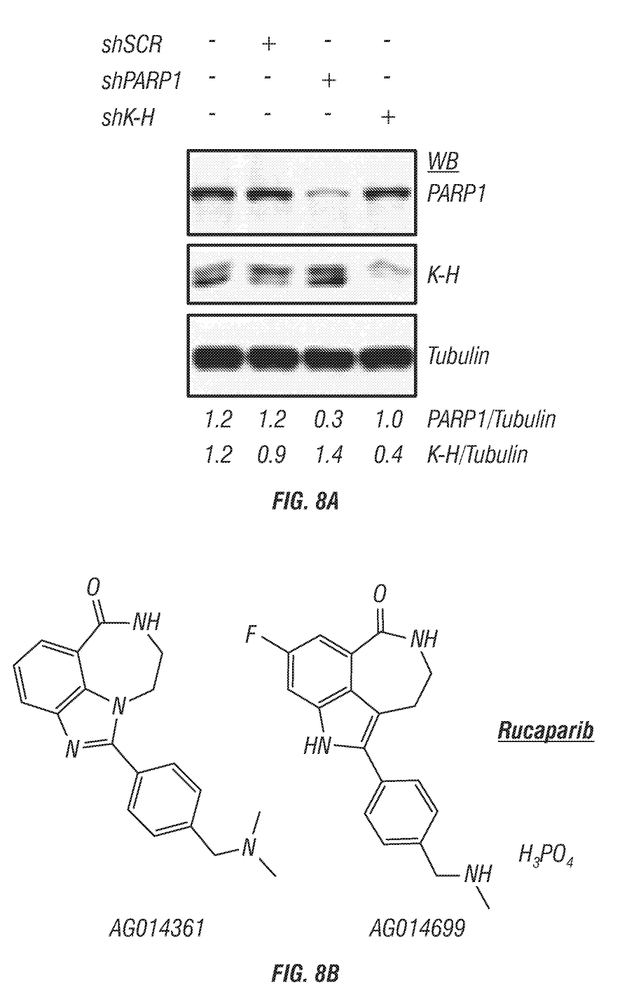

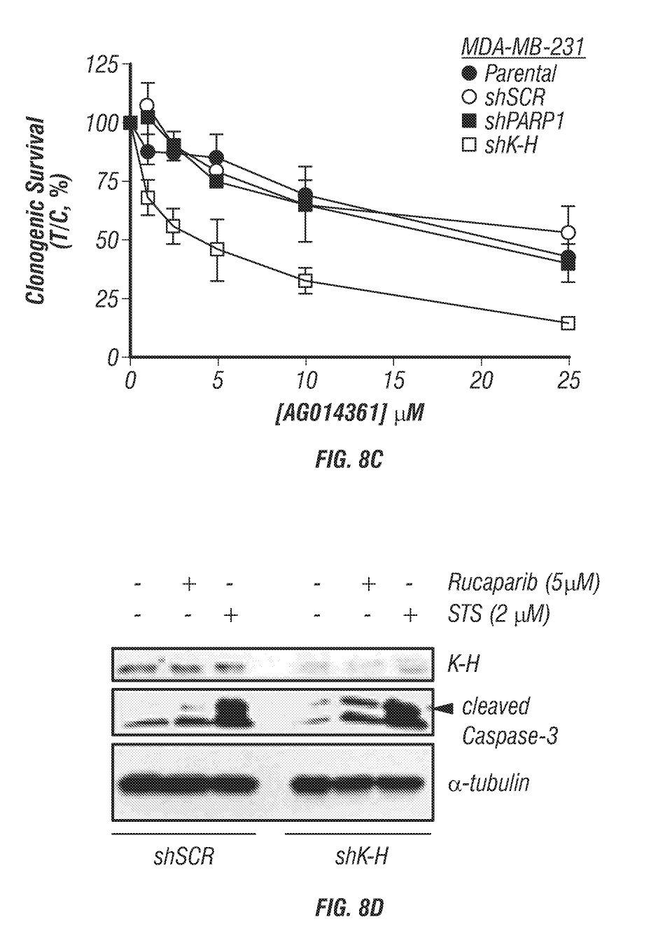

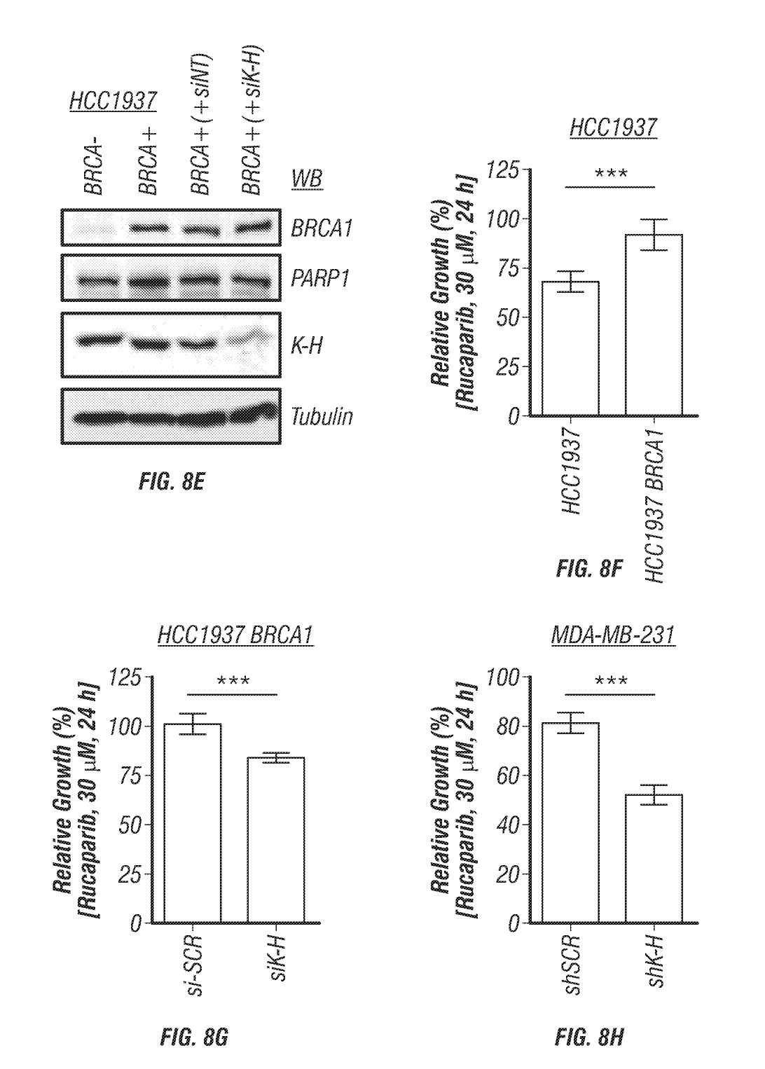

The inventors previously showed that loss of K-H expression led to increased formation of R-loops, DSBs and an accompanying defect in Artemis-mediated NHEJ repair capacity (Morales et al., 2014). The inventors reasoned that shK-H-depleted cells may be hypersensitive to PARP1 inhibition, since synthetic lethality may result from two dysfunctional DNA repair mechanisms (Ashworth, 2008 and Banerjee et al., 2010)—a phenotype analogous to BRCA mutations that are highly sensitive to PARP1i (Bryant et al., 2005 and Farmer et al., 2005). To test this, they generated stable K-H or PARP1 shRNA knockdown clones of BRCA-proficient MDA-MB-231 (hereafter, 231) TNBC cells. A significant elevation of PARP1 activity was observed in stable shK-H-depleted 231 cells (˜3-fold higher compared to shSCR, FIG. 1A); whereas, PARP1 activity was attenuated in stable shPARP1 cells (˜3-fold lower compared to shSCR, FIG. 1A). Depletion of K-H stimulated PARP1 activity in a manner similar to BRCA-deficient cancers, which underlies their hypersensitivity to PARP1i (Gottipati et al., 2010). Elevated PARP1 may minimize the toxic effects of intrinsic DSBs formed in shK-H cancer cells, allowing for survival. Successful stable knockdown of protein expression in shK-H and shPARP1 cells was validated by western blot analyses compared to shSCR cells (FIG. 8A).

The inventors then tested whether pharmacological ablation of PARP1 activity induced synthetic lethality in BRCA-proficient, shK-H-deficient 231 cells. Using colony forming assays (CFAs), the inventors found that shK-H cells were more sensitive to the PARP1i, AG014361 (Calabrese et al., 2004), compared to parental or shSCR 231 cells (FIGS. 8B-C). Importantly, treatment of PARP1-deficient (shPARP1) 231 cells with AG014361 did not induce additional lethality attributed by the lack of target specificity (i.e., other than PARP1), as shown by the dose-response curve coinciding with parental or shSCR cells (FIG. 8C).

The inventors then evaluated the effects of Rucaparib/AG014699 (FIG. 1B), a newer and more potent version of AG014361 currently in clinical trials (Thomas et al., 2007 and Plummer et al., 2008). Treatment of shK-H 231 cells with Rucaparib showed ˜6-fold greater lethality than similar AG014361 exposures (0.66 vs 3.7 M, respectively) (FIG. 1B and FIG. 8C). This -fold difference was comparable to prior preclinical results that established that Rucaparib was more potent than AG014361 (Thomas et al., 2007) in killing BRCA-deficient breast cancers. In contrast, comparable LC₅₀ values (˜20 μM) were obtained in parental, shSCR, or shPARP1 231 cells for either PARP1i under similar treatment conditions (FIG. 1B and FIG. 8C). Further analyses of cell death in Rucaparib-treated shK-H 231 cells showed a dose-dependent increase in apoptosis (FIG. 1C), confirmed by caspase-3 activation (cleavage) with staurosporine (STS)-exposed 231 cells used as a positive control (FIG. 8D). For comparison, the inventors confirmed that the lethality of Rucaparib-treated BRCA-deficient HCC1937 was abolished after reconstitution with BRCA1 cDNA. Importantly, depletion of K-H expression using siK-H treatment of BRCA1-corrected HCC1937 cells induced lethality after exposure to Rucaparib, comparable to genetically matched Rucaparib-exposed BRCA1-deficient cells expressing normal K-H protein levels (FIGS. 8E-H). Overall, these results suggest that a synthetic lethal relationship exists between PARP1 inhibition and shK-H loss in BRCA-proficient breast cancer cells.

Genetic Silencing of Both K-H and PARP1 is Synthetic Lethal.

The inventors next examined whether genetic suppression of PARP1 activity in shK-H-depleted cells might have similar effects as PARP1i (vide supra) for target validation. Successful depletion of PARP1 via siRNA-mediated knockdown (siPARP1) in shK-H 231 cells or depletion of K-H using siK-H in shPARP1 231 cells are shown in FIGS. 1D and 1F. Relative to untreated control, siK-H or siPARP1 alone showed little or no lethality. In contrast, depletion of both proteins together resulted in a stark reduction in colony formation (FIGS. 1E and 1G). Knocking down K-H in shPARP1 231 cells (FIG. 1H) induced more apoptosis in a time-dependent manner (FIG. 1I); whereas, depletion of either K-H or PARP1 alone showed no statistically significant cell death (FIG. 1H-I). Collectively, these findings confirm that K-H deficiency may be linked with the synthetic lethality due to PARP1 inhibition.

Sensitivity of K-H-Depleted Cells to PARP1i is Mediated by Persistent DSBs.

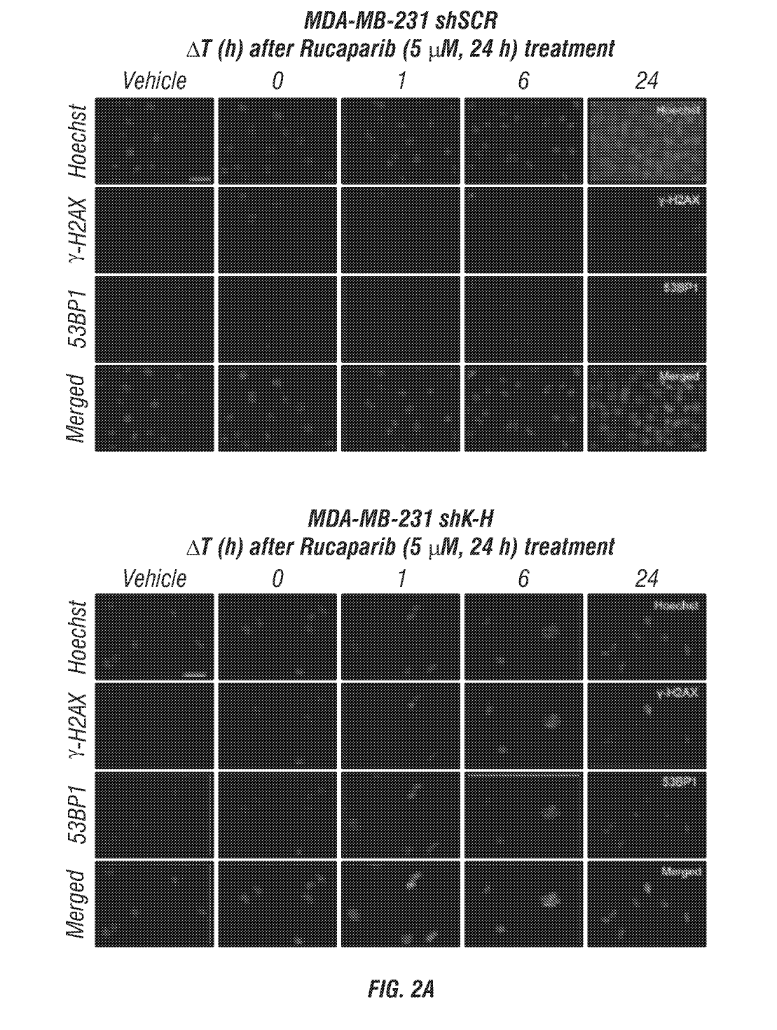

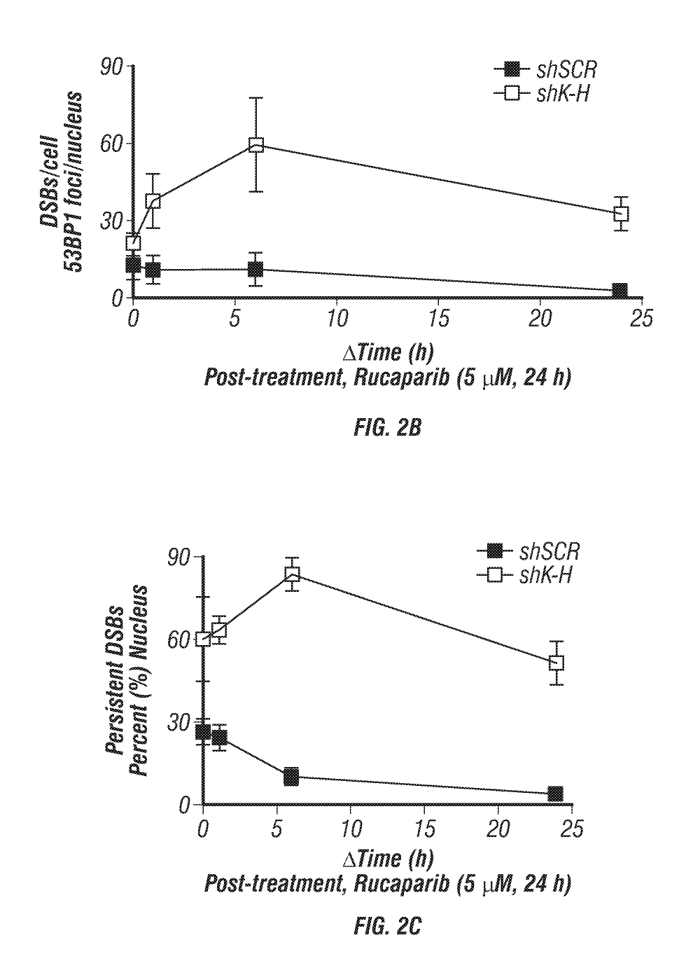

To elucidate the mechanism by which K-H depletion sensitizes cells to PARP inhibition, the inventors treated shSCR vs shK-H 231 cells with vehicle (0.01% DMSO) or Rucaparib (5 μM) for 24 hr and quantified DSB formation at various times post-treatment using γ-H2AX and/or 53BP1 as surrogate DSB markers (Lobrich et al., 2010; Anderson et al., 2001 and Bekker-Jensen et al., 2005) (representative images, FIG. 2A). Dramatic increase in DSB levels were formed in shK-H-depleted 231 cells over time as indicated by 53BP1 foci formation (FIGS. 2A-B). More importantly, the levels of co-localized γ-H2AX and 53BP1 foci (FIGS. 2A and 2C), indicative of persistent DSBs, dramatically increased after Rucaparib treatment, as exposed shK-H-depleted cells try to amplify the signal to recruit essential repair factors to DSB sites for survival (Asaithamby et al., 2008; Karlsson, 2004). In contrast, shSCR 231 cells with wild-type K-H expression contained significantly lower levels of DSBs after Rucaparib exposure, and were resistant to the damaging effects, as the rate of DNA repair was not grossly compromised. Collectively, these observations suggest that the sensitivity of shK-H-deficient cells to PARP inhibition could be due to the creation and inefficient repair of toxic DSBs.

Loss of K-H Compromises CDK1 Expression/Activity and Reduces HR-Mediated DSB Repair.

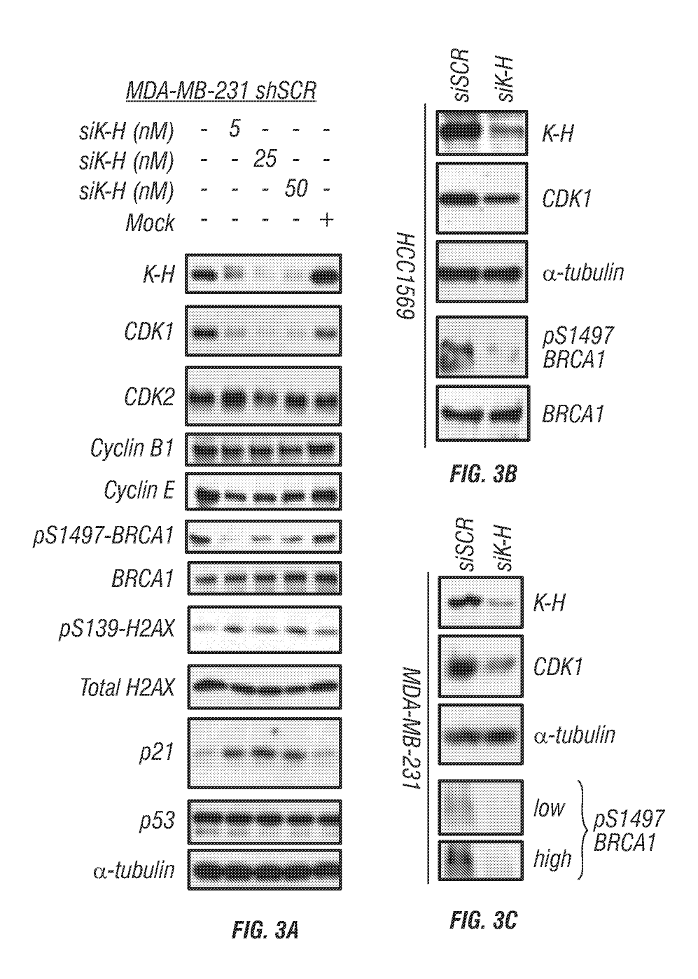

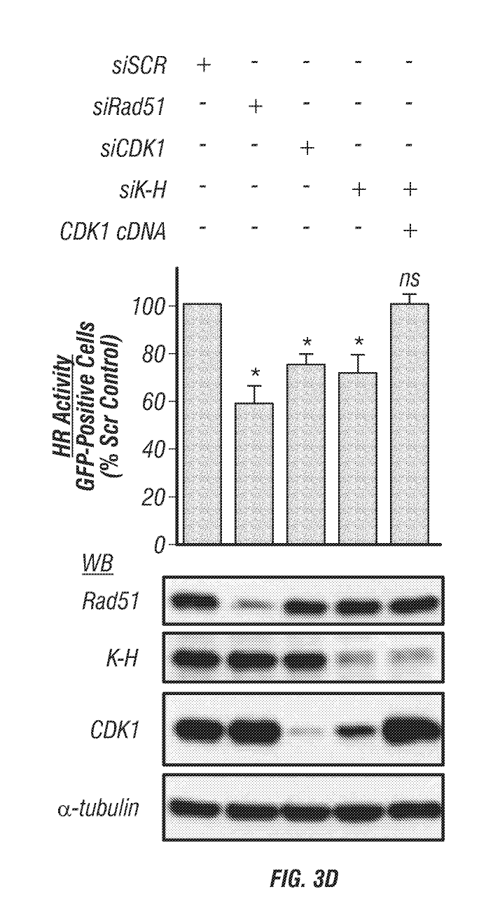

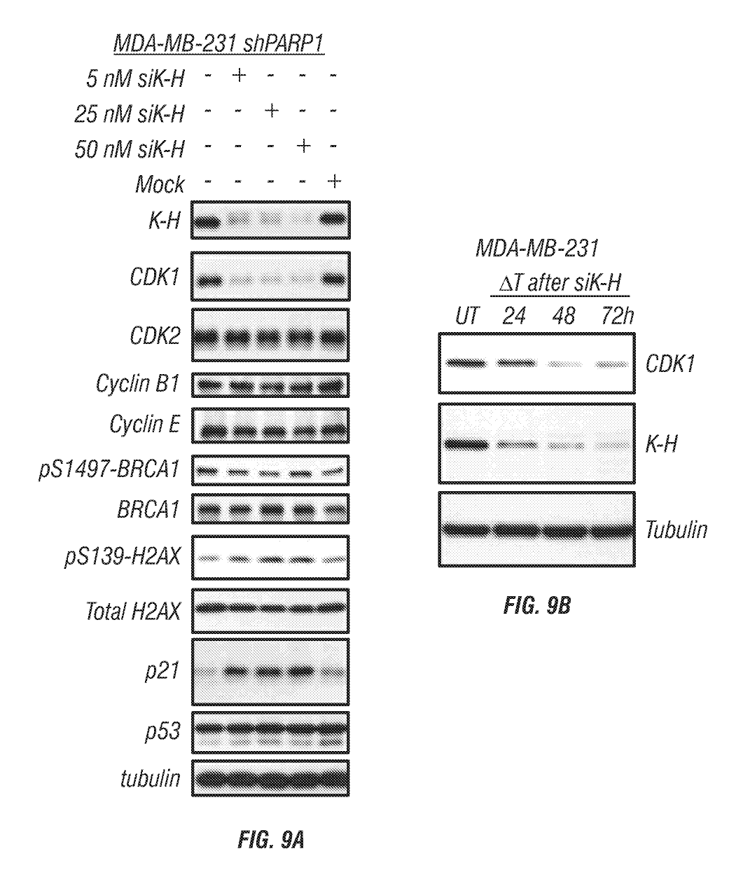

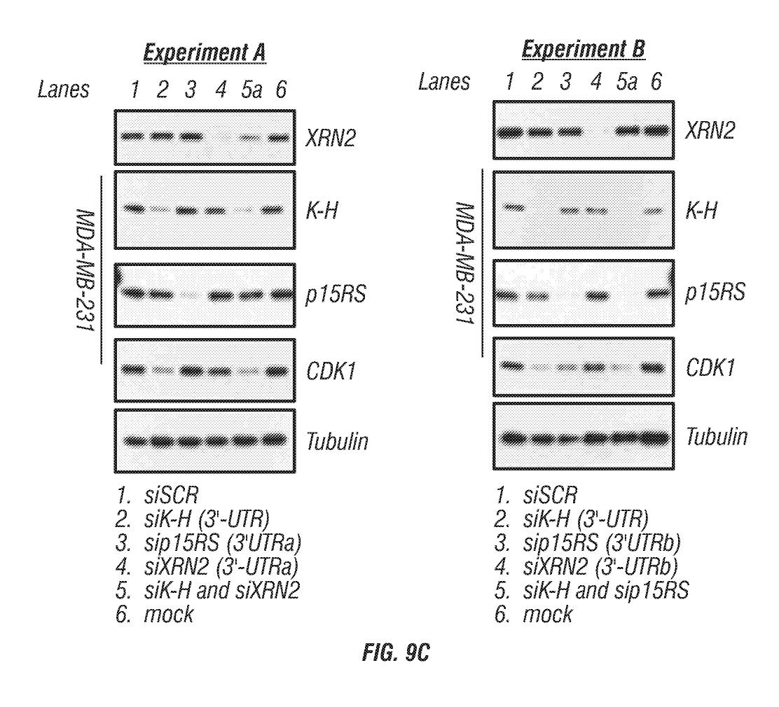

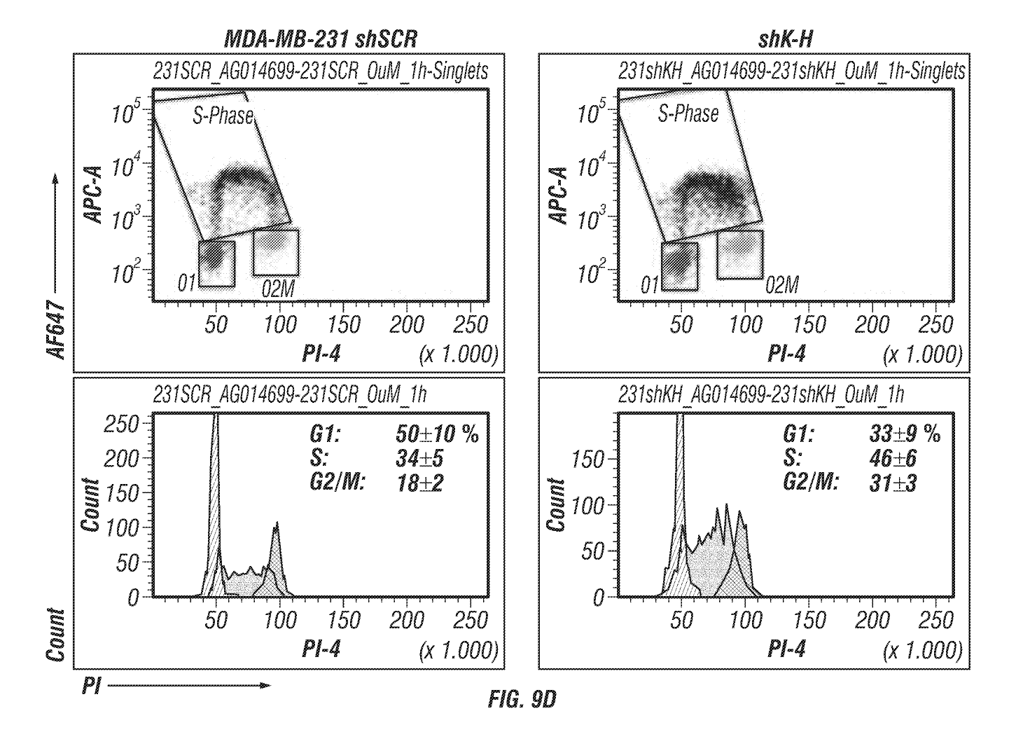

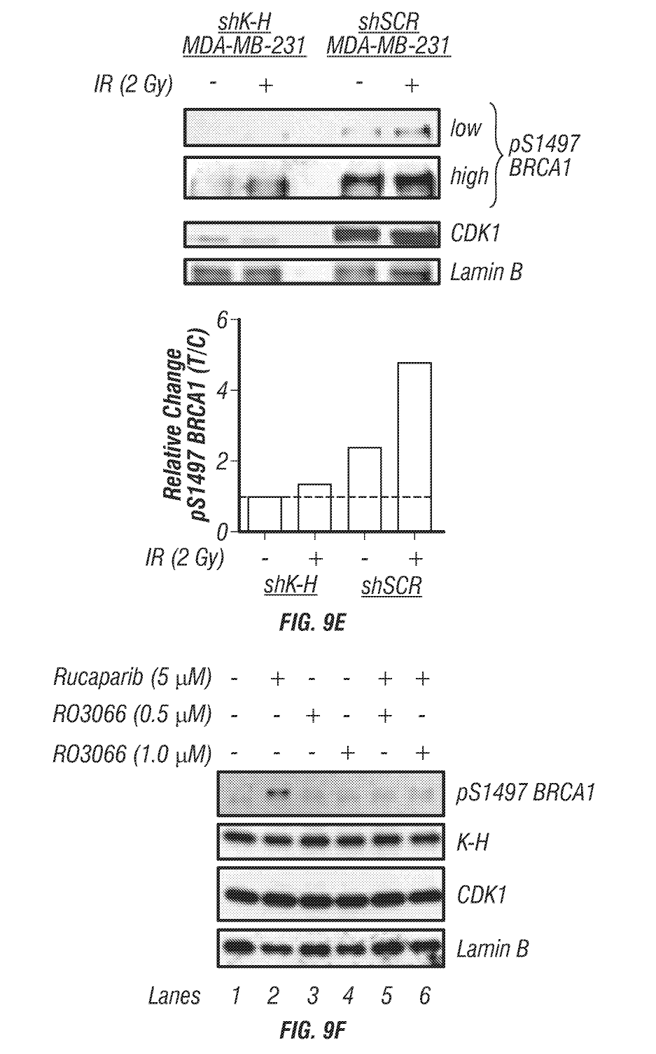

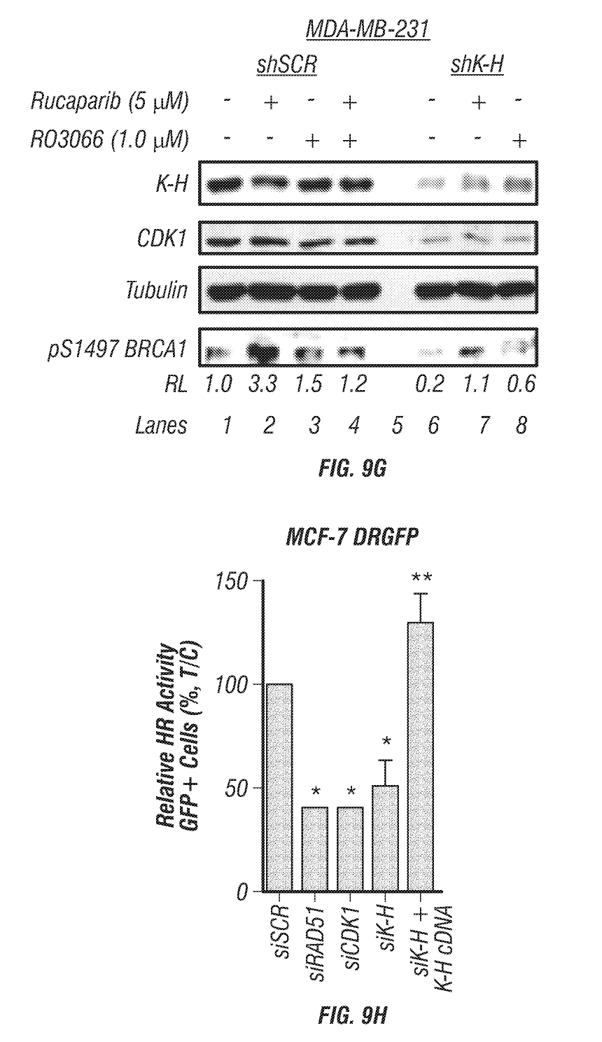

The hypersensitivity of K-H deficient cells to PARP inhibition implies a potential role of K-H in homologous recombination (HR), possibly by regulating gene transcription. Since K-H/RPRD1B was recently shown to regulate cell-cycle-related genes (Lu et al., 2012), the inventors searched for crucial cell-cycle related factors known to regulate HR function, whose expression might be concomitantly decreased upon K-H depletion. By immunoblot analyses, the inventors observed a slight reduction in cyclin E expression during siK-H dose-response knockdown, consistent with prior reports (Lu et al., 2012). The inventors did, however, noted a stark reduction in CDK1, but not CDK2, protein levels in several cell lines (FIGS. 3A-C and FIG. 9A), something not been reported elsewhere. Interestingly, CDK1 and K-H protein levels positively correlated with each other in a dose and time-dependent manner as noted in transient siK-H knockdowns for up to 72 hr (FIGS. 3A-C and FIG. 9B). Importantly, CDK1 regulation by K-H knockdown was specific since siRNA depletion of PARP1 or XRN2 (a transcription termination factor known to associate with K-H (Morales et al., 2014)) showed no apparent CDK1 protein reduction (FIGS. 9A and 9C); importantly, knockdown of p15RS, a close homolog and known binding factor of K-H (Ni et al., 2014), also did not affect steady state levels of CDK1 or K-H (FIG. 9C). CDK1 is the master regulator of the core cell cycle machinery that can execute all events required for the mammalian cell cycle (Santamaria et al., 2007). Indeed, a short-term S-G2/M cell cycle defect was noted in shK-H-depleted asynchronous cells (FIG. 9D), validating prior reports (Kittler et al., 2007). Additionally, CDK1 plays crucial roles in transcription (Chymkowitch and Enserink, 2013 and Chymkowitch et al., 2012), mitochondrial metabolism (Wang et al., 2014), and HR-mediated DSB repair by phosphorylating BRCA1 for proper function (Johnson et al., 2011). Indeed, the inventors noted an impairment of BRCA1 phosphorylation (pS1497) in siK-H-depleted 231 cells, but not in untreated or siSCR-treated controls (FIGS. 3A-C and FIG. 9A). This was also the case after exposure to ionizing radiation (IR) (FIG. 9E) or Rucaparib (FIGS. 9F-G). Furthermore, exogenous insults did not cause CDK1 protein reduction (FIGS. 9F-G). Interestingly, p53-independent activation of p21 and phosphorylation of H2AX (pS139) were also noted only in shK-H-depleted cells, even in the absence of exogenous insult (FIG. 3A, and FIG. 9A).

Down-regulation of CDK1 protein levels by K-H loss prompted the inventors to investigate a possible functional connection between K-H and HR-dependent DSB repair. Using the DR-GFP HR repair reporter system (Pierce et al., 1999 and Bolderson et al., 2010), they found that depletion of K-H significantly reduced the rate of HR comparable to known effects of siRAD51 and siCDK1 (FIG. 3D). Importantly, re-expression of CDK1 in K-H deficient cells rescued the HR defect. Similar results were found in MCF-7 breast cancer cells, where additional re-expression of siRNA-resistant K-H cDNA in siK-H-depleted cells also rescued HR capacity (FIG. 9H), suggesting a specific functional defect in HR was not an off-target effect. Together, these results suggested that K-H-facilitated expression of CDK1 is required for efficient HR-mediated DSB repair, and the role of K-H in stabilizing Artemis (Morales et al., 2014) is dispensable as a mechanism for synthetic lethality when combined with PARP1 inhibition. Consistent with this notion, K-H depletion sensitized cancer cells to treatment with PARP inhibitors (vide supra) in concordance with reports that a deficiency in CDK1 (Johnson et al., 2011 and Turner et al., 2008), but conceivably not Artemis (Lord et al., 2008), renders cells hypersensitive to PARP1 inhibition.

K-H as Potential Functional Biomarker for PARP1i Synthetic Lethality in BRCA-Proficient Breast Cancers.

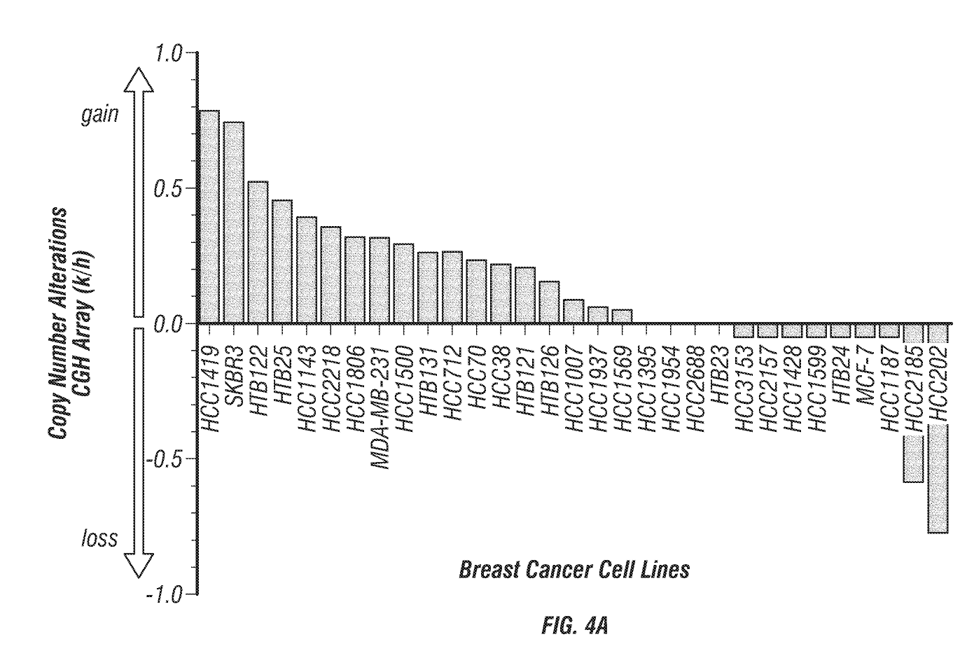

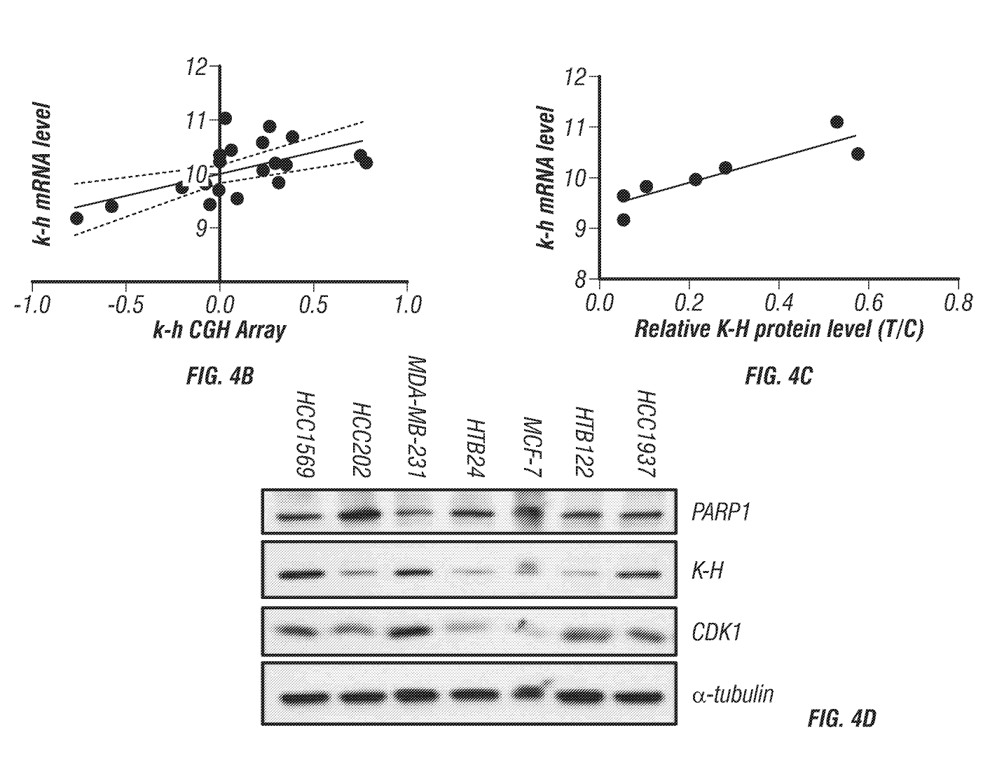

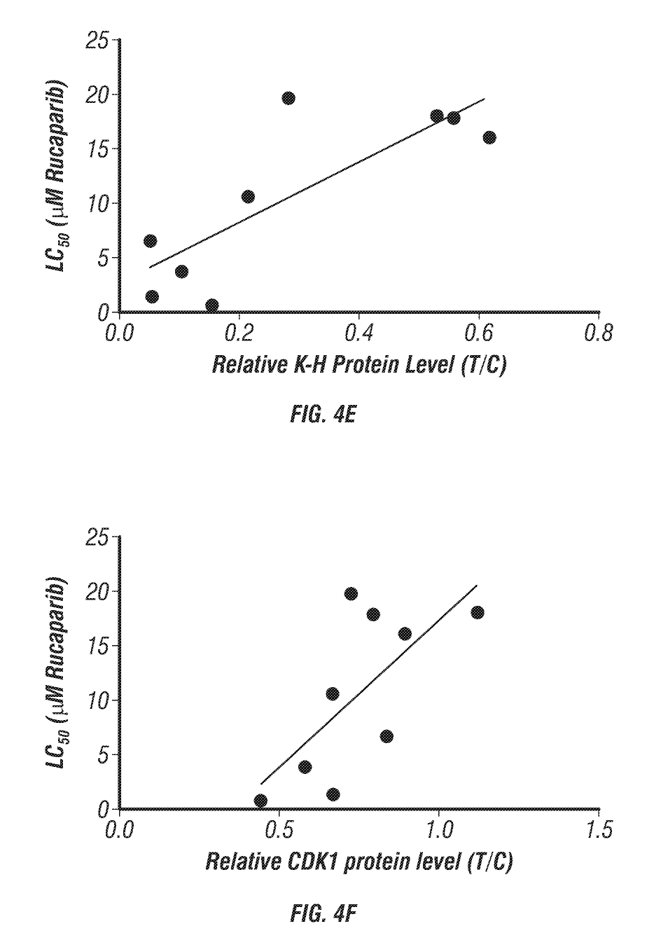

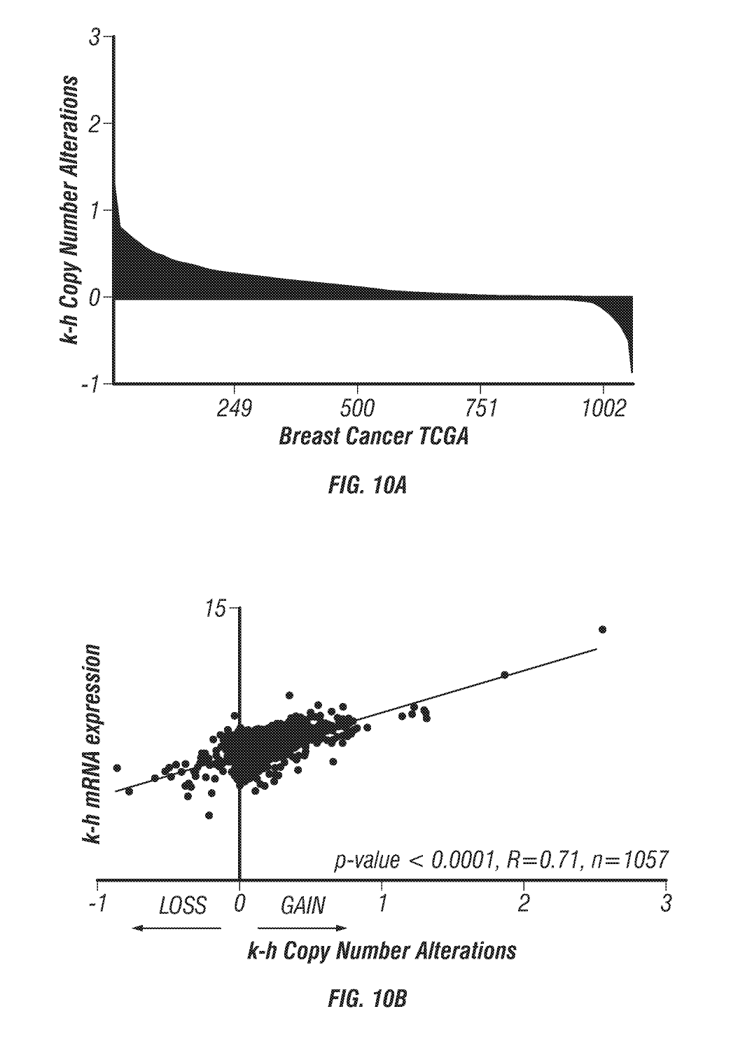

The inventors then investigated whether K-H expression could potentially be utilized as a functional biomarker to predict the hypersensitivity of BRCA-proficient cells to PARP inhibition. The inventors analyzed k-h copy number alterations in a panel of breast cancer cell lines (FIG. 4A) by Comparative Genomic Hybridization (CGH) array data (Kao et al., 2009). Approximately 55% of BRCA-proficient cancer cell lines showed variable gains in k-h copy number, whereas, ˜29% show variable k-h copy number loss (compared to 6% BRCA-deficient cells). These results closely mimic k-h CNA and mRNA expression data obtained from The Cancer Genome Atlas gathered from breast cancer patients (FIGS. 10A-B). Further analysis of k-h mRNA levels in these cell lines showed significant correlation with their corresponding aCGH values (p=0.0089, FIG. 4B) and K-H protein levels (p=0.006, FIG. 4C). Importantly, K-H protein levels generally correlated with CDK1 expression by immunoblot (FIG. 4D), consistent with K-H regulation of CDK1 expression. A subset of BRCA-proficient cancer cells with varying K-H/CDK1 levels were then treated with various Rucaparib doses and survival assessed by clonogenic assays (Table S1). Cells with lower K-H/CDK1 levels showed were hypersensitive to Rucaparib (lower LC₅₀ values) compared to cells with higher K-H/CDK1 levels. In fact, a significant positive correlation was obtained when examining Rucaparib LC₅₀ vs K-H (p=0.008, FIG. 4E) or CDK1 protein levels (p=0.038, FIG. 4F). These results suggest that endogenous K-H loss in BRCA-proficient cancers are accompanied by concomitant loss of CDK1 and inefficient BRCA1 phosphorylation and HR function, allowing use of K-H levels as a biomarker for PARP1i synthetic lethality.

K-H Promotes CDK1 Transcription by Recruiting RNAPII.

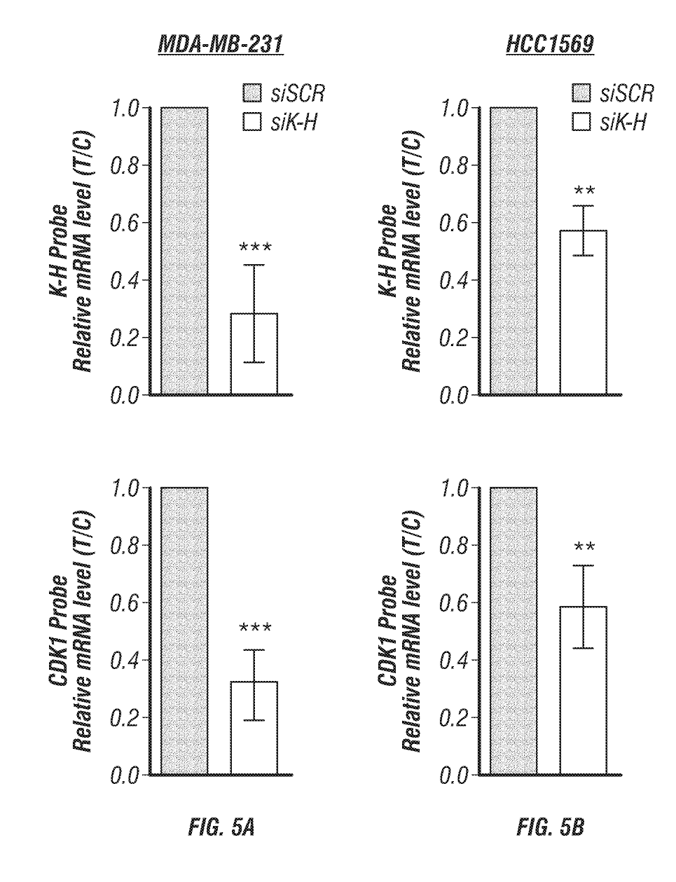

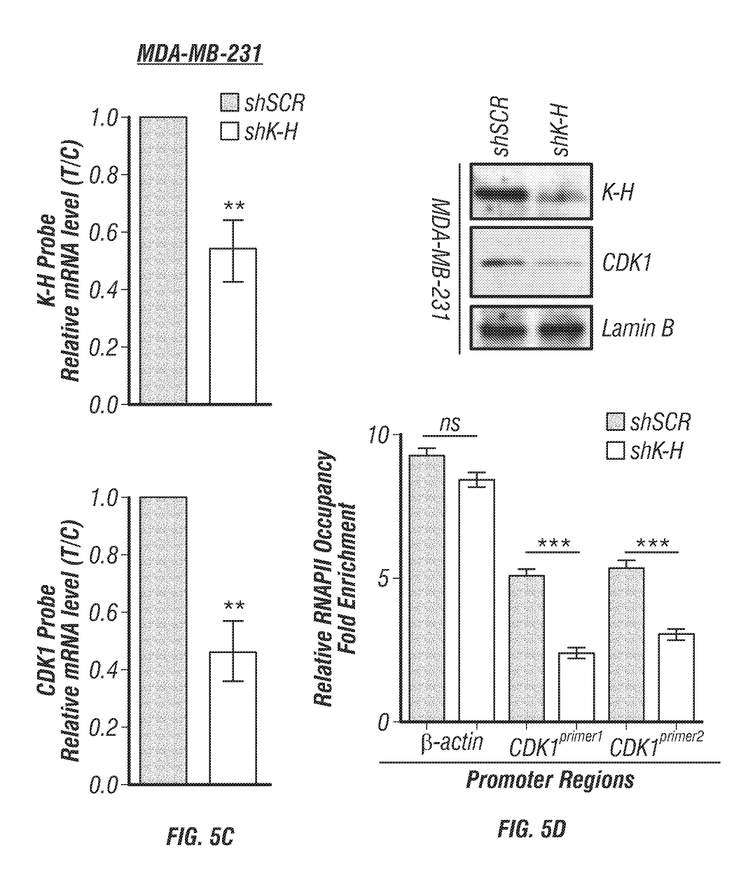

Since K-H depletion resulted in a concomitant reduction in CDK1 protein level in various cell lines (FIGS. 4A-F and FIG. 8A), the inventors investigated the mechanism underlying K-H regulation CDK1 levels. The inventors measured CDK1 mRNA expression in 231 (FIG. 5A) and HCC1569 (FIG. 5B) cells after transient knockdown of K-H via siRNA directed to its 3′-UTR. CDK1 mRNA levels were dramatically reduced suggesting involvement of K-H in CDK1 transcription. Similar results were found in shK-H 231 cells, where knockdown was achieved by shRNA specific to the K-H coding region (FIG. 5C). Prior studies have suggested that K-H interacts with RNAPII in vivo and in vitro (Lu et al., 2012; Ni et al., 2011), but the mechanistic and dynamic basis for this interaction has yet to be firmly established. The inventors then examined the ability of K-H to mediate localization of RNAPII to the specific promoter region of CDK1, using actin as a control in chromatin immunoprecipitation (ChIP) assays (FIG. 5D). Depletion of K-H resulted in a stark reduction of RNAPII occupancy at the CDK1 promoter region (FIG. 5D). In contrast, RNAPII localization at the actin promoter was virtually unaffected. These results strongly suggested that K-H does not universally affect gene transcription, but promotes transcription of specific genes (e.g., CDK1) by efficiently recruiting RNAPII to specific promoter regions.

K-H Residue R106 is Essential for RNAPII CTD (pS2) Binding to Promote CDK1 Transcription Via Recruitment to the CHR Promoter Element.

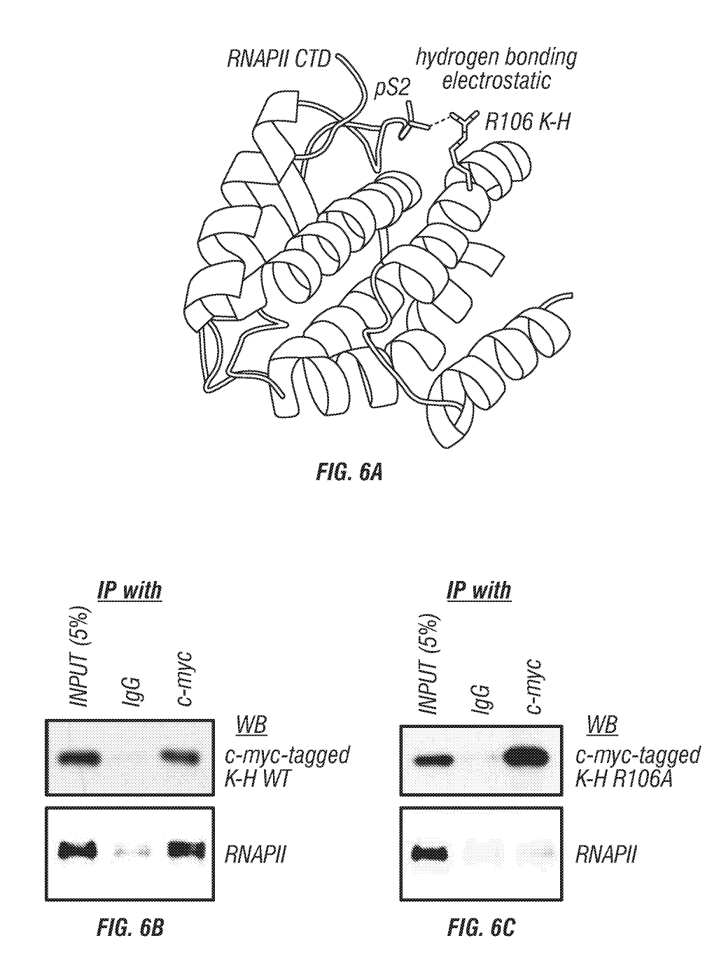

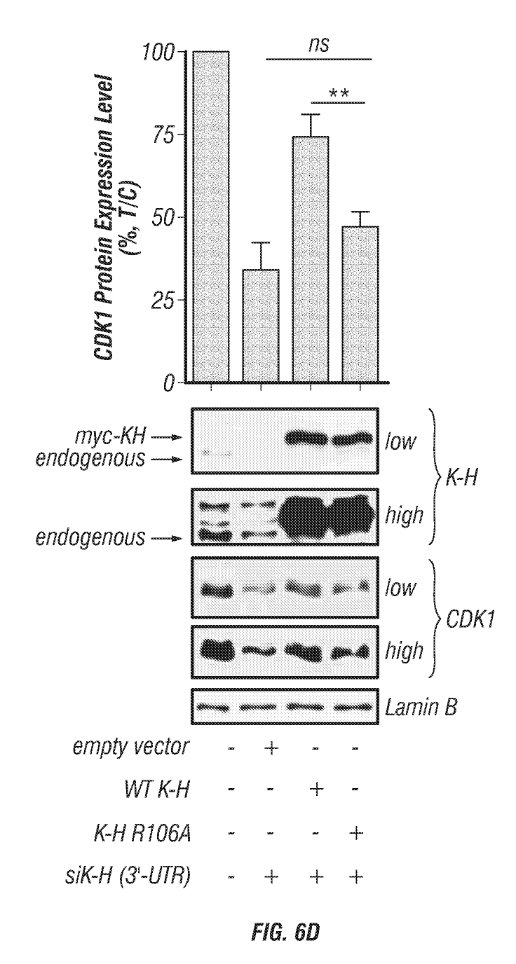

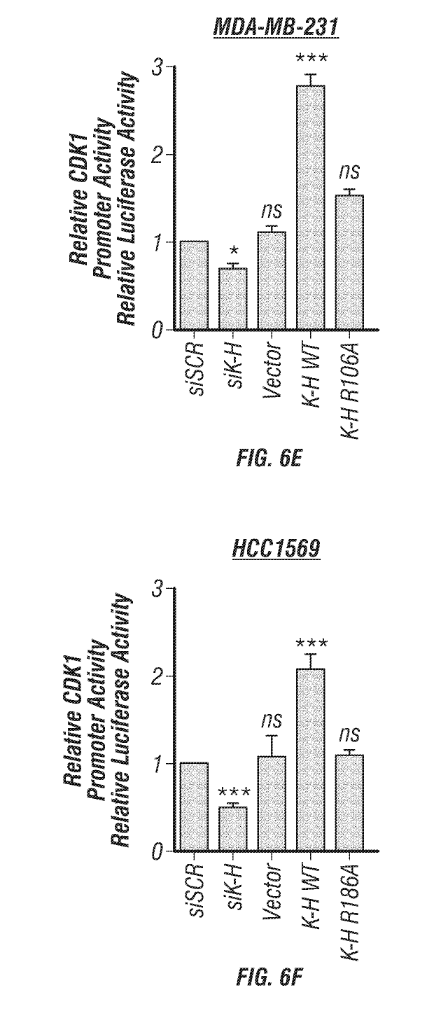

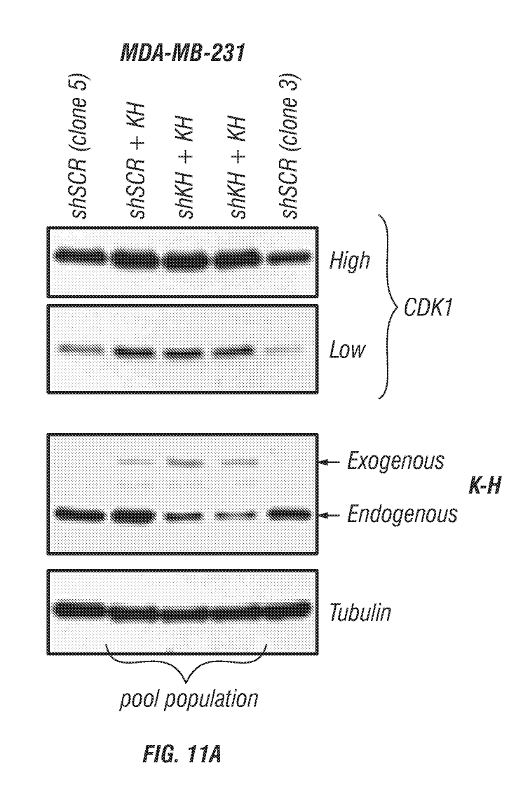

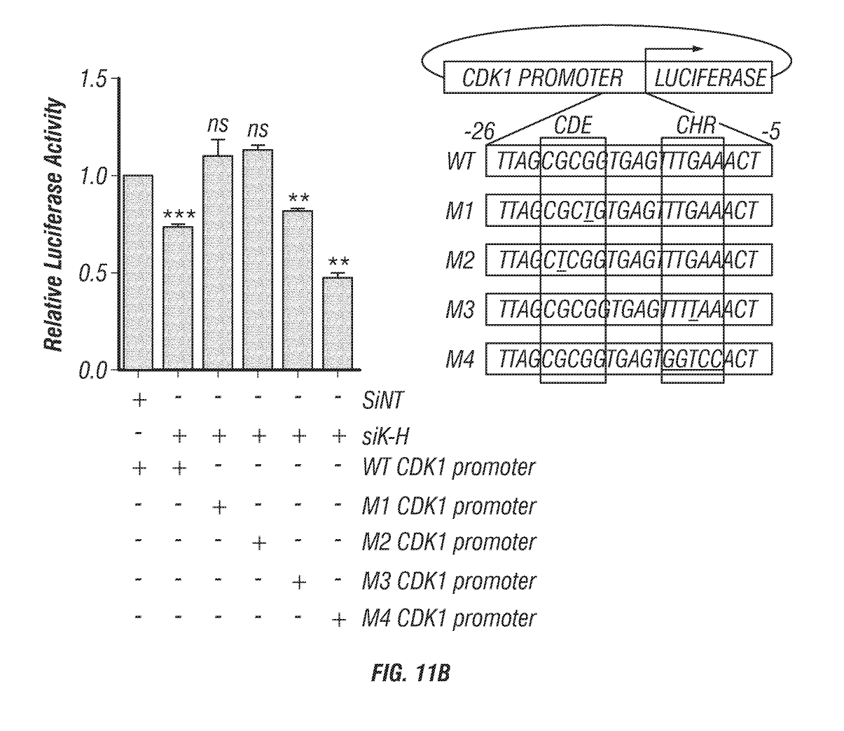

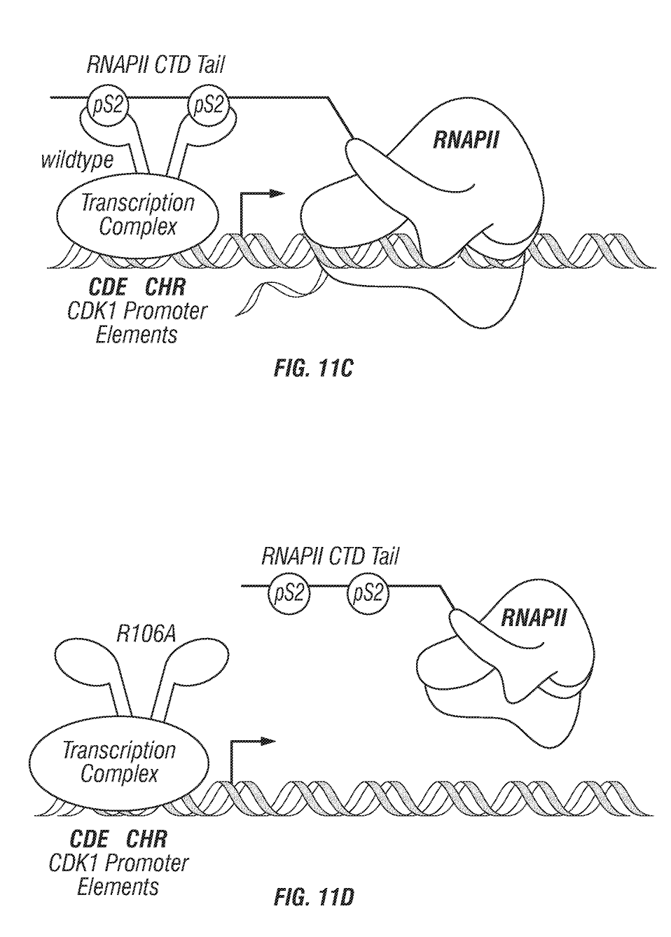

To further examine the importance of K-H binding to RNAPII in regulating CDK1 transcription, the inventors generated a point mutation in K-H believed to abrogate RNAPII binding. Using homology modeling studies with crystallized RTT103 (Lunde et al., 2010), a yeast homolog of K-H, they identified arginine 106 (R106) as a crucial K-H residue with specific and strong affinity to phospho-Ser 2 (pS2) containing C-terminal domain (CTD) of RNAPII via hydrogen bonding and electrostatic interactions (FIG. 6A). Recent structural studies supported this result (Ni et al., 2014). Using immunoprecipitation experiments, the inventors confirmed that myc-tagged wild-type K-H pulled down RNAPII (FIG. 6B), whereas, mutating the K-H residue to alanine (R106A) abrogated RNAPII binding (FIG. 6C) due to loss of crucial interactions. Rescue experiments in K-H-depleted cells using transient 3′-UTR siRNA showed that re-expression of wild-type K-H cDNA rescued CDK1 protein expression to nearly that of untreated control 231 cells (FIG. 6D). In contrast, re-expression of empty pCMV vector or R106A mutant K-H failed to rescue CDK1 loss (FIG. 6D). Interestingly K-H over-expression in shK-H 231 cells also concomitantly rescued CDK1 protein levels (FIG. 11A), while over-expression in shSCR cells resulted in increases in CDK1 levels beyond basal levels (FIG. 11A), consistent with the correlation of K-H and CDK1 levels in breast cancer cell lines (FIGS. 4A-F). Thus, CDK1 loss after K-H knockdown appears to be regulated at the transcriptional level and was not due to off-target effects.

The mechanism for how K-H and RNAPII interaction regulated CDK1 transcription was then examined using a 3 kb portion of the human CDK1 gene promoter regulating firefly luciferase (Itzhaki et al., 1997 and Badie et al., 2000). The inventors noted that CDK1 promoter activity was significantly reduced upon siK-H knockdown vs siSCR treatment in 231 cells (FIG. 6E), consistent with quantitative mRNA data (FIGS. 5A-B). Furthermore, over-expression of mutant R106A K-H also failed to stimulate CDK1 promoter activity. In contrast, over-expression of wild-type K-H significantly enhanced CDK1 promoter activity (˜3-fold). Similar experiments using HCC1569 cells (FIG. 6F) yielded comparable results suggesting that K-H binding to pS2-containing CTD of RNAPII is crucial to efficiently promote CDK1 transcription (FIG. 11B).

The inventors then defined the specific conserved sequence motif within the CDK1 promoter region by which K-H promotes CDK1 transcription. Two major elements, the cell-cycle dependent element (CDE) and cell-cycle gene homology region (CHR) motifs are known to promote CDK1 transcription (Badie et al., 2000). The inventors utilized a series of plasmids containing wild-type or mutations of CDE or CHR sites within the CDK1 promoter (FIG. 6G). Over-expression of K-H in shK-H 231 cells significantly enhanced wild-type CDK1 promoter activity vs vector alone (FIG. 6G). In contrast, point mutation(s) in the CHR element of the CDK1 promoter effectively blocked CDK1 promoter activity mediated by K-H vs the wild-type CHR domain-containing CDK1 promoter (FIG. 6G). In contrast, point mutations in the conserved CDE sequence had little influence on K-H-driven CDK1 promoter activity, suggesting that this sequence motif was dispensable. In fact, further reduction in CDK1 promoter activity was observed in CHR mutants, but not in CDE mutants, in transient siK-H knockdown 231 cells (FIG. 11C), which further suggests that the CHR site is crucial for K-H function in driving the CDK1 promoter.

Re-Expression of Wild-Type K-H in shK-H-Deficient Cells Rescues Cdk1 Expression and Reverses Synthetic Lethality with Rucaparib.

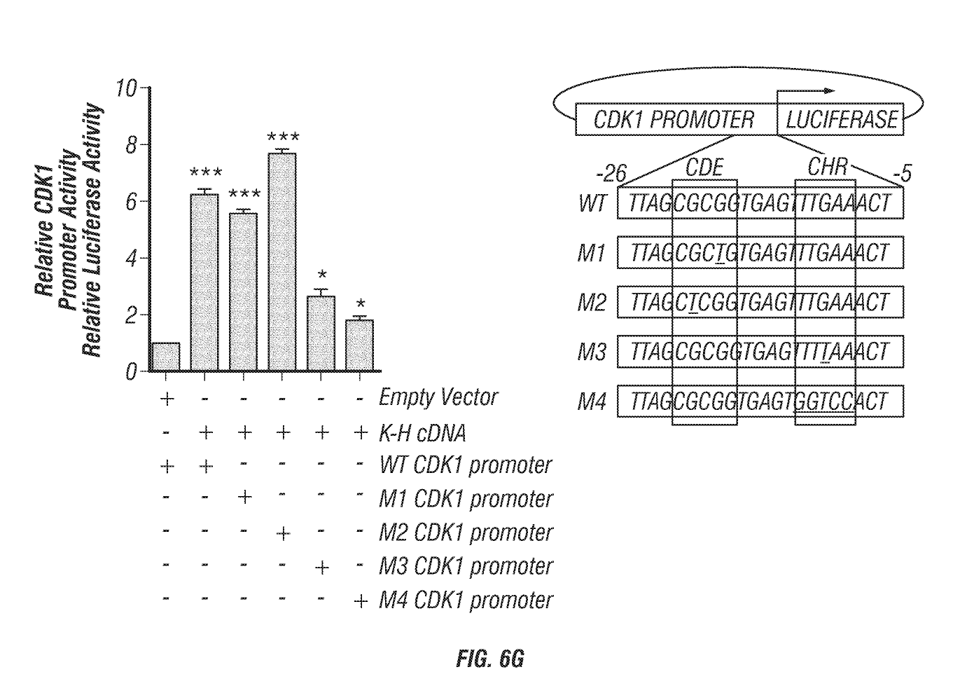

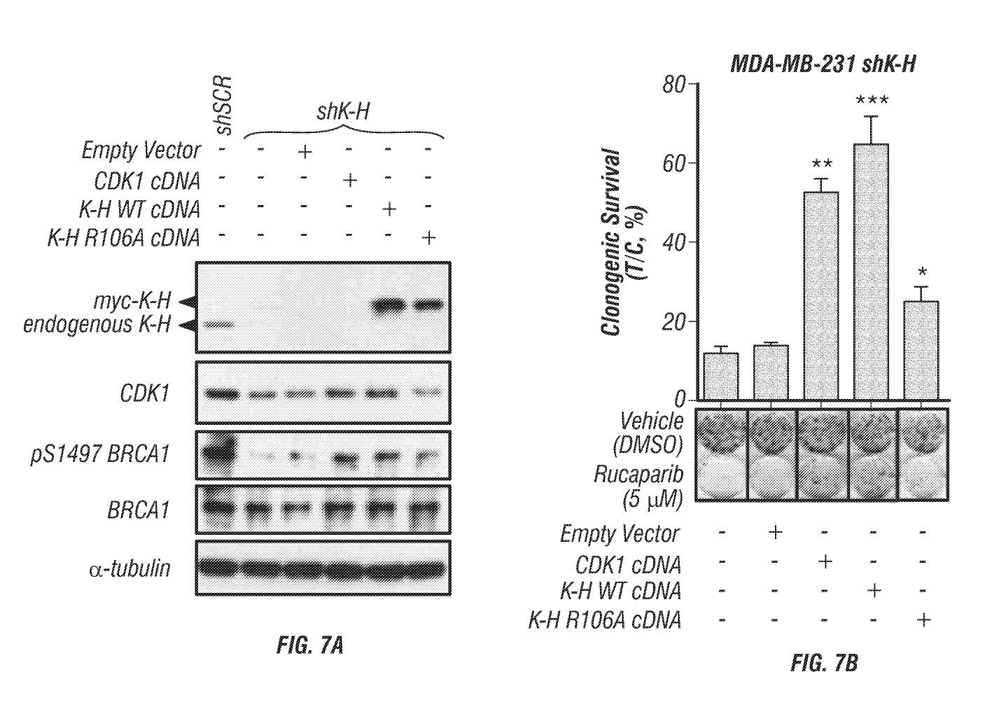

Loss of K-H promotes a state of “BRCAness” in cancer cells by compromising the transcription (FIGS. 6A-G) and activity (FIGS. 3A-D, pS1497 BRCA1) of CDK1, which phosphorylates BRCA1 for proper HR function (34). The inventors explored whether sensitivity to a PARP1i (Rucaparib) could be rescued by re-expression of either wild-type K-H, CDK1, or mutant R106A K-H in stable shK-H 231 BRCA-proficient cells (FIG. 7A). Depletion of K-H concomitantly decreased to CDK1, causing hypersensitivity to Rucaparib (FIG. 7B). Re-expression of wild-type K-H rescued CDK1 protein expression and dramatically increased the survival of Rucaparib-treated shK-H 231 cells. Interestingly, re-expression of CDK1 cDNA in shK-H deficient 231 cells also rescued lethality, suggesting that the ability of K-H to promote CDK1 expression was crucial for resistance to PARP inhibition. In contrast, re-expression using R106A mutant K-H, which ineffectively rescued CDK1 promoter activity (FIGS. 6A-G) and protein expression (FIG. 7A) only partially rescued Rucaparib-exposed stable shK-H cells. Re-expression of shK-H cells using empty vector did not alter synthetic lethality caused by Rucaparib exposure. These findings suggest that K-H may serve as a novel functional biomarker for identifying BRCA-proficient tumors that are hypersensitive to PARP1i.

As described, the preceding data demonstrate an unannotated role for K-H (RPRD1B) in promoting the transcription of cyclin-dependent kinase 1 (CDK1). This transcriptional regulation is facilitated by the ability of K-H to bind and localize RNAPII to a specific region within the CDK1 gene promoter. Since K-H associates with RNAPII, the inventors sought to determine whether overall mRNA expression in cells depleted for K-H using stable shRNA knockdown would yield similar transcriptome profile obtained for cells with BRCA1, RAD51, or BRIT1 deficiencies, collectively termed the “HR deficiency (HRD)” gene signature (Peng et al. 2014). Ingenuity Pathway Analyses (IPA) of altered mRNA expression in BRCA-proficient MDA-MB-231 (hereafter 231) cells stably depleted for K-H expression revealed enrichment in the following top five molecular and cellular pathways: (1) cell cycle, (2) cell death and survival, (3) cellular growth and proliferation, (4) cellular assembly and organization, and (5) DNA replication, recombination, and repair (FIG. 13). This spectrum of altered gene expression was analogous to the top signature pathways found for HRD in cancer cells (Peng et al., 2014). Consistent with the theory that K-H depletion affects HR function, we noted that in 231 cells depleted for K-H expression, CDK1, a major cell cycle and DNA damage response and repair (DDR) effector, was significantly repressed. This was intriguing since CDK1 expression remains putatively constant in cells (Dalton 1992; Welch and Wang 1992). Loss of CDK1 could lead to a downstream impairment of HR-mediated repair of DSBs by compromising BRCA1 phosphorylation for efficient recruitment of RAD51 to sites of DSBs (Johnson et al. 2011).

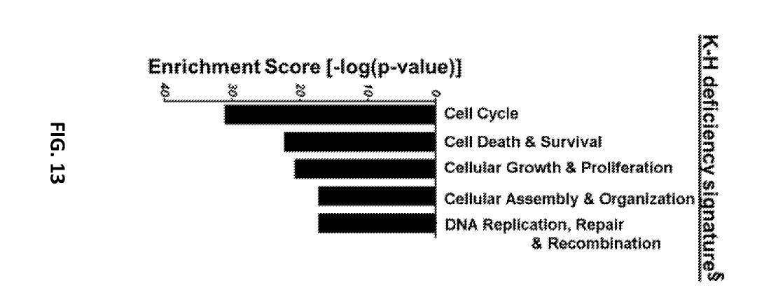

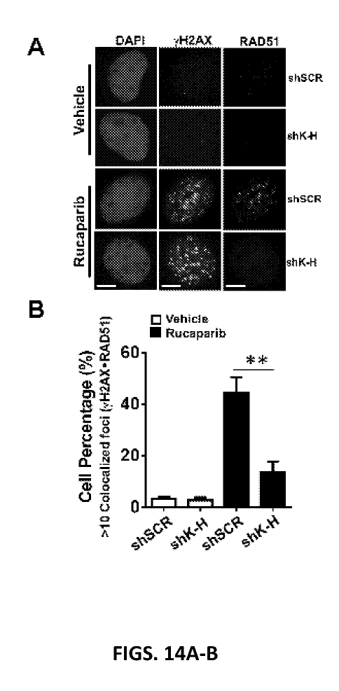

The inventors further validated that K-H depletion compromised RAD51 foci formation after Rucaparib treatment (FIGS. 14A-B). This is consistent with impaired downstream HR function caused by loss of CDK1 that phosphorylates and activates BRCA1 (e.g., S1497) required for efficient RAD51 recruitment. Collectively, these observations suggest that the hypersensitivity of shK-H-deficient cells to PARP inhibition could be due to the creation and inefficient repair of toxic DSBs generated by persistent R-loops resulting from K-H loss (Morales et al., 2014), PARP inhibition, and HR deficiency.

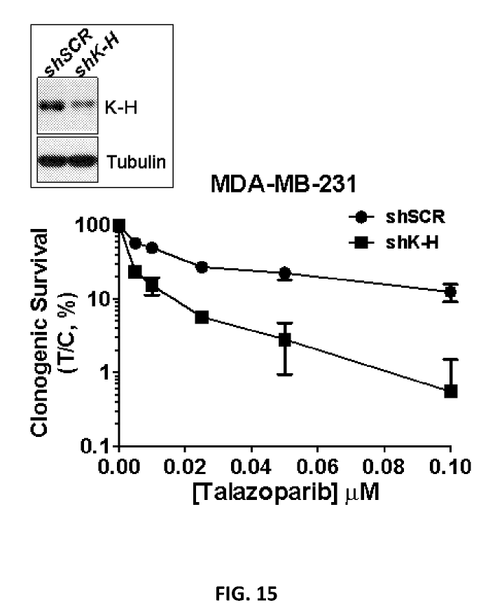

In FIG. 15, the inventors tested the ability of BMN-673 (Talazoparib) to induce synthetic lethality in BRCA-proficient, shK-H-deficient 231 cells using colony forming assays (CFAs). Talazoparib is a PARP inhibitor that is currently in Phase III Clinical Trial. As expected, they found that shK-H cells were more sensitive to the PARP inhibitor, Talazoparib, compared to the more resistant shSCR 231 control cells.

TABLE S1
Summary of breast cancer cell lines and LC₅₀ values
obtained in the study

| | | | | | | AG014699 | |
|---|---|---|---|---|---|---|---|
| | | | | | K-H | Mean | |
| Breast Cancer | | | | | protein | LC₅₀ (SEM) | |
| Cell Line | ER* | PR* | Her2* | p53* | level | (μM) | |
| HCC1569 (A) | neg | neg | pos | −^{M} | 0.69 | 18 | (3) |
| MDA-MB-231 (B) | neg | neg | neg | ++^{M} | 0.46 | 20 | (5) |
| MCF-7 (L) | pos | pos | neg | +/−^{WT} | 0.20 | 11 | (2) |
| HCC202 (L) | neg | neg | pos | − | 0.18 | 6.7 | (1) |
| HTB24 (B) | neg | neg | neg | −^{M} | 0.14 | 1.5 | (0.1) |
| HTB122 (B) | neg | neg | neg | ++^{M} | 0.09 | 3.9 | (1.1) |
| HCC1937 [symbol] (A) | neg | neg | neg | [−] | 0.45 | ND^{‡} | |
| MDA-MB-231 | neg | neg | neg | ++^{M} | 0.51 | 18 | (5) |
| shSCR (B) | | | | | | | |
| MDA-MB-231 | neg | neg | neg | ++^{M} | 0.56 | 17 | (3) |
| shPARP1 (B) | | | | | | | |
| MDA-MB-231 | neg | neg | neg | ++^{M} | 0.15 | 0.7 | (0.1) |
| shK-H (B) | | | | | | | |

Abbreviations: A = Basal A subtype;
B = Basal B subtype;
L = Luminal subtype;
(−) = protein not expressed;
(+) = protein expressed;
M = mutant form;
WT = wild type form
*Determined from ⁴
AG014699 is also known as Rucaparib. Mean LC₅₀ values were obtained from long term colony forming assays. (SEM) denotes standard error of mean from three replicates. [symbol]
BRCA-mutant breast cancer cell line.
^{‡}ND means not determined due to inability to perform robustly in a clonogenic assay.

TABLE S2
Summary of antibodies used in the study

| | | | Dilution |
|---|---|---|---|
| | SOURCE | Cat# | (WB, IF, IP) |
| PRIMARY | | | |
| ANTIBODY | | | |
| RPRD1B (K-H) | Sigma | SAB2701444 | 1:1000 |
| PARP1 | Sta. Cruz | F-2, sc-8007 | 1:1000 |
| CDK1 (CDC2) | Sta. Cruz | C-19, sc-954 | 1:1000 |
| pS1497-BRCA1 | Sta. Cruz | Sc-12889-R | 1:500 |
| CDK2 | Sta. Cruz | M2, sc-163 | 1:500 |
| BRCA1 | CalBioChem | OP92 | 1:500 |
| pS139-H2AX | Thermo | MA1-2022 | 1:1000, 1:500 |
| | Scientific | | |
| 53BP1 | Sta. Cruz | H300, Sc- | 1:500 |
| | | 22760 | |
| H2AX | Bethyl | A300-082A | 1:1000 |
| | Laboratories | | |
| Cyclin E | Sta. Cruz | HE12, sc-247 | 1:500 |
| Cyclin B1 | Sta. Cruz | H-20, sc-594 | 1:500 |
| Cyclin D1 | Sta. Cruz | H-295, sc-753 | 1:500 |
| p21 | Sta. Cruz | F-5, sc-6246 | 1:500 |
| p53 | Sta. Cruz | DO-1, sc-126 | 1:1000 |
| cleaved Caspase-3 | Cell Signaling | 9664 | 1:500 |
| poly(ADP)-ribose | Trevigen | 4335-mc-100 | 1:1000 |
| (PAR) | | | |
| c-myc | Sigma | 9E10 | 1:1000, |
| | | | 2 μg (IP) |
| RNA Polymerase II | Bethyl Lab | A300653A | 1:500 |
| | Sta. Cruz | N-20, sc-899 | 2 μg (ChIP) |
| α-tubulin | Sigma | T9026 | 1:25,000 |
| Lamin B | Sta. Cruz | G1, sc-373918 | 1:500 |
| p15RS | Sta. Cruz (m) | FF-8, sc-81849 | 1:1000 |
| | Sta. Cruz (r) | L-16, sc-85090 | 1:1000 |
| XRN2 | Abcam | 72181 | 1:500 |
| Rad51 | Sta. Cruz | H-92, sc-8349 | 1:1000 |
| SECONDARY | | | |
| ANTIBODY | | | |
| Control mouse IgG | Sta. Cruz | sc-2025 | 2 μg (IP) |
| Control rabbit IgG | Sta. Cruz | sc-2027 | 2 μg (ChIP) |
| Goat anti-mouse HRP | Sta. Cruz | sc-2031 | 1:5000-50,000 |
| Goat anti-rabbit HRP | Sta. Cruz | sc-2030 | 1:2000-5000 |
| Goat anti-mouse Alexa | Life | A11001 | 1:1000 |
| Fluor 488 | Technologies | | |
| Donkey anti-rabbit | Life | A21207 | 1:1000 |
| Alexa Fluor 594 | Technologies | | |

TABLE S3
Summary of siRNA sequences used in the study

| siRNA TARGET | Custom siRNA sequence or Cat. # | Source |
|---|---|---|
| siSCR | 5′-GACCGUUAGGUACAGAAGAUU-3′ (SEQ ID NO: 6) | Dharmacon |
| siK—H (3′-UTR) | 5′-GGCUGAAACCCAAACUAUAUU-3′ (SEQ ID NO: 7) | Dharmacon |
| siK—H (pool) | ON-TARGET PLUS (#L-013787-01-0005) | Dharmacon |
| sip15RS (3′UTRa) | 5′-GCCCAUGGGCUAUCAACUU[dT][dT]-3′ (SEQ ID NO: 8) | Sigma |
| sip15RS (3′UTRb) | 5′-GUUAAGCAGUGCCACAUA[dT][dT]-3′ (SEQ ID NO: 9) | Sigma |
| siXRN2 (3′-UTRa) | 5′-GGAUCUGAAUUUAUUAUUG[dT][dT]-3′ (SEQ ID NO: 10) | Sigma |
| siXRN2 (3′-UTRb) | 5′-GGAAUAGGAGUACUGGUUU[dT][dT]-3′ (SEQ ID NO: 11) | Sigma |
| siPARP1 (pool) | ON-TARGET PLUS (#L-006656-03-0005) | Dharmacon |
| siRAD51 (ORF) | SASI_Hs02_00553645 | Sigma |
| siCDK1 (ORF) | SASI_Hs01_00044049 Seq. Start 865 | Sigma |

TABLE S4
Summary of shRNA sequences used in this study

| | | |
|---|---|---|
| MISSION | 5′CCGGGCAAGATGTTTCTCTATTGGACTCGAGTCCAATAGAG | Sigma |
| pLKO.1puro | AAACATCTTGCTTTTTTG-3′ (SEQ ID NO: 12) | |
| shK-H (coding region) | | |
| MISSION | 5′-CCGGGCGCGATAGCGCTAATAATTTCTCGAGAAATTATTA | Sigma |
| pLKO.1puro | GCGCTATCGCGCTTTTT-3′ (SEQ ID NO: 13) | |
| shSCR | | |

TABLE S5
Chromatin Immunoprecipitation (ChIP) primers

| TARGET PROMOTER | Primer Sequence | Source |
|---|---|---|
| ACTIN (f) | 5′-CTCAATCTCGCTCTCGCTCT-3′ (SEQ ID NO: 14) | Sigma |
| ACTIN (r) | 5′-CTCGAGCCATAAAAGGCAAC-3′ (SEQ ID NO: 15) | Sigma |
| CDK1 primer1 (f) | 5′-CCTCTTTCTTTCGCGCTCTA-3′ (SEQ ID NO: 16) | Sigma |
| CDK1 primer 1 (r) | 5′-GGACCCCGTTCCTCAATACT-3′ (SEQ ID NO: 17) | Sigma |
| CDK1 primer 2 (f) | 5′-TTTTTCTCTAGCCGCCCTTT-3′ (SEQ ID NO: 18) | Sigma |
| CDK1 primer 2 (r) | 5′-CTCTCCGCTCAATTTCCAAG-3′ (SEQ ID NO: 19) | Sigma |

TABLE S6
Site-directed mutagenesis primers

| Site | Primer Sequence | Source |
|---|---|---|
| K—H | 5′-GAAGGCTGTAAAAAACCTTTAGAAGCATTGCTGAACATCTG | Sigma |
| R106A Forward | GCAAGAAC-3′ (SEQ ID NO: 20) | |
| K—H | | |
| R106A Reverse | 5′-GTTCTTGCCAGATGTTCAGCAATGCTTCTAAAGGTTTTTTA | Sigma |
| | CAGCCTTC-3′ (SEQ ID NO: 21) | |

### Example 3—Discussion

The inventors present compelling data illustrating a functional role for K-H in promoting transcription and expression of CDK1, a crucial factor in cell cycle progression, particularly G1/S and G2/M transitions (Malumbres and Barbacid, 2009; Hochegger et al., 2007; Bashir and Pagano, 2005). CDK1 is involved in a myriad of other biological processes, such as direct phosphorylation of transcription factors involved in cell cycle progression (Landry et al., 2014), regulation of electron transport complex I for mitochondrial respiration (Wang et al., 2014), HR factors involved in DNA resection (Huertas et al., 2008; Ira et al., 2004 and Chen et al., 2011) and BRCA1 phosphorylation needed for efficient HR-mediated DSB repair. While CDK1 kinase activity is dispensable for its ability to recruit the proteasome to the site of active transcription (Chymkowitch et al., 2012) its overall protein level is required for this function as well. CDK1 regulation at transcriptional and protein expression levels in normal cells typically remain stable throughout the cell cycle to support crucial biological processes for faithful cell division (Castedo et al., 2002). The question of how aberrant CDK1 expression is regulated at the transcriptional level, especially under pathological conditions (e.g., cancer), has yet to be firmly defined. These studies strongly suggest that K-H is intimately involved in a putative complex residing at the CHR promoter region of CDK1 to efficiently promote transcription (FIG. 7C(i)). The inventors noted a direct correlation between K-H and CDK1 protein levels in a majority of breast cancer cell lines (FIG. 3D) with the exception of HTB122 cell line suggesting other unknown compensatory pathways that warrant further investigation. At the transcriptional level, they observed that the CDK1 gene responded rapidly to variations in K-H protein expression. Aberrant K-H over-expression greatly enhanced CDK1 transcription, whereas, K-H knockdown in 231 or HCC1569 breast cancer cell lines concomitantly reduced CDK1 transcription and protein expression (FIGS. 6E-G). Mechanistically, they demonstrate a functional role for K-H in stimulating CDK1 promoter activity via its binding to RNAPII pS2 CTD heptapeptide repeats (FIGS. 6E-F and 7C(i)) and facilitating interactions of RNAPII with the CHR domain of the CDK1 promoter. Based on a recent study (28), and in conjuction with these results, the inventors speculate that K-H predominatly forms a homodimer, especially in K-H-overexpressing cancer cells. This dimer binds two nearby pS2 sites in RNAPII CTD heptapeptide repeats to provide a docking scaffold for other factors, such as RPAP2 (RNA Polymerase II Associated Protein 2) (Ni et al., 2014). This interaction could then promote efficient dephosphorylation of Ser-5 (pS5) in the RNAPII CTD tail to initiate the transition from initiation to elongation of CDK1 transcription by RNAPII.

In addition to CDK1, cyclin E was also downregulated after K-H knockdown, consistent with a prior report (Lu et al., 2012). The inventors also noted that transient siK-H-depleted or shK-H 231 cells exhibited a short-term S-G2/M arrest, which they attribute to CDK1 loss. This is consistent with previously validated studies where K-H was interestingly identified on a genome-scale search as a novel gene that induced G2/M cell cycle arrest upon knockdown with endoribonuclease-prepared short interfering RNA (esi-RNA) that has lower off-target effects than single or pooled siRNAs (Kittler et al., 2007). These observations suggest a role for K-H in regulating other CHR-containing genes with the conserved sequence, TTGAA (e.g., CDK1 but not CDK2), and this has implications toward the design and development of novel single or combined anticancer agent approaches for personalized therapy. Such an innovative strategy could target the malignant activity, or inherent resistance of aberrant K-H over-expression in cancer cells.

Similar to yeast RTT103, K-H also plays a role in transcription termination (Morales et al., 2014). Loss of K-H showed increased formation of persistent DNA:RNA hybrid structures (R-loop), where upon collision with the replisome or transcription-coupled nucleotide excision factors could spontaneously collapse to R-loop-driven DSBs (Aguilera and Garcia-Muse, 2012; Wimberly et al., 2013; Sollier et al., 2014). A recent study suggested the critical involvement of BRCA1 in a DNA repair mechanism that resolves R-loop-associated genomic instability (56). It is, therefore, conceivable that compromised BRCA1 phosphorylation due to aberrant K-H deficiency could potentially explain the observed increase in the formation of γ-H2AX (FIG. 3A and FIG. 9A), but a more in-depth mechanistic analysis is warranted. Nonetheless, DSBs formed as a result of replication fork collapse were documented to be predominantly repaired by HR (Karanam et al., 2012). Here, the inventors showed that HR was defective in K-H depleted cells, which sheds light on the surprising upsurge in PARP1 enzyme activity required for survival. This is consistent with the link between K-H deficiency and synthetic lethality caused by ablation of PARP1 activity by genetic silencing or PARP1i (e.g., Rucaparib) treatment.

Another major aspect of this work is that K-H appears to be a predictive biomarker for PARP1i lethality (FIG. 7C). Examination of breast cancer TCGA data suggest that a subset of BRCA-proficient cancer patients with aberrant K-H deletion would benefit from PARP1i treatments. Based on the inventors' studies using 231 genetic models and a panel of breast cancer cell lines, this effect is due an HR deficiency resulting from the concomitant loss of CDK1 expression and activity, which is absolutely required to phosphorylate BRCA1 and other HR factors (Huertas et al., 2008; Ira et al., 2004; Chen et al., 2011) for proper DSB repair (FIG. 7C(ii)). Indeed, PARP1i lethality directly correlated with K-H expression in several BRCA-proficient breast cancer cell lines tested here (FIG. 4E). Aberrant over-expression of K-H was linked to PARPi resistance; while reduction of K-H potentiated the lethality of PARP1i due to persistent and most likely complex DSBs. The inventors' previous study showed that Artemis expression was lost due to K-H's ability to mediate protein-protein stability of this complex DSB repair protein (Morales et al., 2014). Since over-expression of CDK1 corrected PARP1 inhibition synthetic lethality, the data suggest that Artemis is dispensable for synthetic lethality with Rucaparib (FIGS. 7A-B). This observation is corroborated by synthetic lethal screening studies in cancer cell lines suggesting that the loss of Artemis showed no hypersensitivity to PARP inhibition (Turner et al., 2008). Thus, these studies provide proof-of-principle suggesting that aberrant K-H deficiency in BRCA-proficient breast cancers may be used as a clinically-relevant functional biomarker to predict synthetic lethality with PARP1i treatments for personalized therapy in breast, as well as other human cancers. Future studies will determine whether expression levels of K-H that mediate HR repair through regulation of CDK1 can serve as determinants of therapeutic strategy and of clinical outcome for patients with BRCA-proficient tumors. In addition, the mechanistic and biological insights on the aberrant function of K-H in cancer progression gained from these studies should lead to the design and development of novel chemical agents that inhibit K-H function or innovative technologies to manipulate K-H expression in cancers with amplification of this gene to further expand the use of PARP inhibitors or selectively potentiate other DNA-damaging agents (e.g., ionizing radiation).

The data in FIGS. 13-15 further validates these inventors' findings. In summary, Kub5-Hera (K-H, RPRD1B) is a haploinsufficient hypomorphic protein whose expression is critical for genomic and chromosomal stability (Morales et al., 2014; Patidar et al., 2016). Here, we show that loss of K-H^{RPRD1B}, a known poly(A) mRNA transcription termination factor, abrogates homologous recombination (HR) by downregulating the transcription of a critical DNA damage response and repair effector, cyclin-dependent kinase 1 (CDK1). Indeed, loss of CDK1 compromised BRCA1 phosphorylation that is crucial for the recruitment of RAD51 to the sites of DSBs during HR repair. Furthermore, K-H-depleted BRCA-proficient breast cancer cells exhibit a ‘BRCAness’ gene signature similar to previously reported HR-deficient cells (i.e., loss of BRCA1, RAD51 or BRIT1) (Peng et al., 2014). Pharmacologic ablation of PARP activity using PARP inhibitor, BMN-673 (Talazoparib), in BRCA-proficient cells with aberrant K-H reduction leads to synthetic lethality. Thus, defective HR in a subset of K-H-deficient tumors represents an exploitable cancer vulnerability to PARP inhibition due to their potential over-reliance on alternative-nonhomologous end joining (alt-NHEJ). Collectively, these results suggest that aberrant K-H alterations may impact overall cellular responses and survival to DNA damage and have clinical implications in cancer.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references, to the extent that they provide exemplary procedural or other details supplementary to those set forth herein, are specifically incorporated herein by reference.
Abbondanzo et al., Am. J. Pediatr. Hematol. Oncol., 12(4), 480-489, 1990.
Aguilera and Garcia-Muse, Molecular cell 46, 115, 2012.
Alderbom et al., Genome Research 10(8): 1249-1258 (2000).
Allred et al., Arch. Surg., 125(1), 107-113, 1990.
Anderson et al., Molecular and cellular biology 21, 1719, 2001.
Asaithamby et al., Radiation research 169, 437, 2008.
Ashworth, Cancer research 68, 10021, 2008.
Ashworth, Journal of clinical oncology, 3785, 2008.
Atherton et al., Biol. of Reproduction, 32, 155-171, 1985.
Audeh et al., Lancet 376, 245, 2010.
Ausubel et al., Current Protocols in Molecular Biology, John Wiley & Sons, N.Y. (2003).
Badie et al., Molecular and cellular biology 20, 2358, 2000.
Banerjee et al., Nature reviews. Clinical oncology 7, 508, 2010.
Bashir and Pagano, Nature cell biology 7, 779, 2005.
Becherel et al., PLoS Genet 9, e1003435, 2013.
Bekker-Jensen et al., The Journal of cell biology 170, 201, 2005.
Bhatia et al., Nature 511, 362-365, 2014.
Bolderson et al., Nucleic Acids Res 38, 1821, 2010.
Brown et al., J. Immunol. Meth., 12; 130(1), :111-121, 1990.
Bryant et al., Nature 434, 913, 2005.
Calabrese et al., Journal of the National Cancer Institute 96, 56, 2004.
Castedo et al., Cell death and diferentiation 9, 1287, 2002.
Chen et al., Genome Research 9(5):492-498, 1999.
Chen et al., Nature structural & molecular biology 18, 1015, 2011.
Church and Gilbert, Proc. Natl. Acad. Sci. USA 81:1991-1995, 1988.
Chymkowitch and Enserink, Transcription 4, 3, 2013.
Chymkowitch et al., Proc. Natl. Acad. Sci. USA 109, 10450, 2012.
Cotton et al., Proc. Natl. Acad. Sci. USA 85:4397-4401, 1985.
Dalton, S. The EMBO Journal 11: 1797-1804, 1992.
De Jager et al., Semin. Nucl. Med. 23(2), 165-179, 1993.
Dholakia et al., J. Biol. Chem., 264, 20638-20642, 1989.
Doolittle and Ben-Zeev, Methods Mol. Biol., 109, :215-237, 1999.
Eckert et al., PCR Methods and Applications 1:17, 1991.
Farmer et al., Nature 434, 917, 2005.
Flavell et al., Cell 15:25, 1978.
Fong et al., The New England journal of medicine 361, 123, 2009.
Geever et al., Proc. Natl. Acad. Sci. USA 78:5081, 1981.
Gottipati et al., Cancer research 70, 5389, 2010.
Guatelli et al., Proc. Nat. Acad. Sci. USA 87:1874, 1990.
Gulbis and Galand, Hum. Pathol. 24(12), 1271-1285, 1993.
Hatchi et al., Molecular cell 57, 636.
Helleday, Molecular oncology 5, 387, 2011.
Hochegger et al., The Journal of cell biology 178, 257, 2007.
Hoeijmakers, Nature 411, 366, 2001.
Huertas et al., Nature 455, 689, 2008.
Ira et al., Nature 431, 1011, 2004.
Itzhaki et al., Nature genetics 15, 258, 1997.
Jackson and Bartek, Nature 461, 1071, 2009.
Johnson et al., Molecular Cell 35, 327-339, 2009.
Johnson et al., Nature Medicine 17, 875-882, 2011.
Kao et al., PloS one 4, e6146, 2009.
Karanam et al., Molecular cell 47, 320, 2012.
Karlsson and Stenerlow, Radiation research 161, 517, 2004.
Khatoon et al., Ann. of Neurology, 26, 210-219, 1989.
Kim and Jinks-Robertson, Nat Rev Genet 13, 204-214, 2012.
King et al., J. Biol. Chem., 269, 10210-10218, 1989.
Kittler et al., Nature cell biology 9, 1401, 2007.
Kittler et al., Nature methods 4, 337, 2007.
Kwoh et al., Proc. Natl. Acad. Sci. USA 86:1173, 1989.
Landegren et al., Science 241:1077, 1988.
Landry et al., The EMBO journal 33, 1044, 2014.
Lobrich et al., Cell cycle 9, 662, 2010.
Lord and Ashworth, Nature 481, 287, 2012.
Lord et al., DNA repair 7, 2010, 2008.
Lu et al., Cancer cell 21, 92, 2012.
Lunde et al., Nature structural & molecular biology 17, 1195-1201, 2010.
Malumbres and Barbacid, Nature reviews. Cancer 9, 153, 2009.
Mattila et al., Nucleic Acids Res. 19:4967, 1991.
McPherson et al., PCR Basics: From Background to Bench, Springer-Verlag, 2000.
Michalak and Jonkers, Journal of mammary gland biology and neoplasia 16: 41, 2011.
Morales et al., PLoS Genetics. Submitted, 2016.
Morales et al., The Health Risks of Extraterrestial Environment—jsc.nasa.gov, 2015
Morales et al., Nucleic Acids Research 42, 4996-5006, 2014.
Motea et al., ACS Chemical Biology 7, 988-998, 2012(a).
Motea et al., Chembiochem: a European journal of chemical biology 14, 489-498, 2013.
Motea et al., Nucleic Acids Research. Submitted., 2016.
Motea et al., Nucleic acids research 40, 2357-2367, 2012(b).
Myers et al., Science 230:1242 (1985).
Nakamura et al., In: Enzyme Immunoassays: Heterogeneous and Homogeneous Systems, Chapter 27, 1987.
Nath and Johnson, Biotechnic. Histochem. 73(1):6-22 (1998).
Ni et al., Nature structural & molecular biology 21, 686-695, 2014.
Ni et al., Transcription 2, 237, 2011.
Orita et al., Proc. Natl. Acad. Sci. USA 86:2766-2770 (1989).
O'Shannessy et al., J. Immun. Meth. 99, 153-161, 1987.
Owens and Haley, J. Biol. Chem., 259, 14843-14848, 1987.
Patidar et al., Nucleic Acids Research, 44, 1718-1731, 2016.
Peng et al., Nature Communications 5: 3361, 2014.
Pierce, et al., Genes & development 13, 2633, 1999.
Plummer et al., Clinical cancer research: an official journal of the American Association for Cancer Research 14, 7917, 2008.
Plummer, Current opinion in pharmacology 6, 364, 2006.
Prince et al., Genome Res. 11:152-162 (2001).
Raca et al., Genet Test 8(4):387-94 (2004).
Remington's Pharmaceutical Sciences” 15th Edition, chapter 33, in particular pages 624-652.
Saiki et al., Nature 324:163-166 (1986).
Sanger et al., Proc. Natl. Acad. Sci. USA 74:5463-5467 (1977).
Santamaria et al., Nature 448, 811, 2007.
Schafer et al., Nat. Biotechnol. 15:33-39 (1995).
Schreiber et al., Nature reviews. Molecular cell biology 7, 517, 2006.
Sheffield et al., Proc. Natl. Acad. Sci. USA 86:232-236 (1989).
Sollier et al., Molecular Cell 56, 777, 2014.
Stoneking et al., Am. J. Hum. Genet. 48:370-382 (1991).
Thomas et al., Mol Cancer Ther 6, 945, 2007.
Tomimatsu et al., Nature Communications 5, 3561, 2014.
Turner et al., The EMBO Journal 27, 1368, 2008.
Tutt et al., Lancet 376, 235, 2010.
U.S. Pat. No. 4,683,195
U.S. Pat. No. 4,683,202
U.S. Pat. No. 5,288,644
U.S. Pat. No. 5,491,224
U.S. Pat. No. 5,776,688
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
U.S. Pat. No. 4,472,509
U.S. Pat. No. 4,683,195
U.S. Pat. No. 4,683,202
U.S. Pat. No. 4,938,948
U.S. Pat. No. 5,021,236
U.S. Pat. No. 5,196,066
U.S. Pat. No. 5,288,644
U.S. Pat. No. 5,491,224
U.S. Pat. No. 5,776,688
U.S. Patent Publication No. 2004/0014095
U.S. Patent Publication No. 2009/0137412
Underhill et al., Genome Res. 7:996-1005 (1997).
Vichai and Kirtikara, Nature Protocols 1, 1112-1116, 2006.
Wang et al., Developmental Cell 29: 217, 2014.
Welch and Wang, Proc. Nat'l Acad. Sci. USA 89: 3093-3097, 1992.
Wheeless et al., Cytometry 17:319-326 (1994).
Wimberly et al., Nature Communications 4, 2115, 2013.
WO 99/57318
Wu and Wallace, Genomics 4:560 (1989).
Wu et al., Genes & development 20, 2383, 2006.

1. A method of treating a cancer patient determined to (a) express low/undetectable levels of Kub-5-Hera (K-H), (b) have reduced K-H gene copy number relative to a normal cell, and/or (c) have a loss/reduction of function mutation in a K-H coding region, such as mutation in a K-H RPR (CID) domain, and/or mutation in K-H coiled-coil domain, comprising administering to said subject a PARP1 inhibitor, ionizing radiation, or a chemotherapeutic agent that induces DNA double-strand breaks.

2. The method of claim 1, further performing the determination of (a) low/undetectable levels of K-H, (b) reduced K-H gene copy number and/or (c) mutation in a K-H coding region.

3. The method of claim 1, wherein said chemotherapeutic agent that induces DNA double-strand breaks is doxorubicin, a Topoisomerase I or II poison, paclitaxel, cisplatin, or gemcitabine.

4. (canceled)

5. The method of claim 1, wherein said cancer is selected from breast cancer, lung cancer, pancreatic cancer, brain cancer, ovarian cancer, head and neck cancer and cervical cancer.

6. The method of claim 5, wherein said breast cancer is a BRCA-proficient breast or pancreatic cancer.

7. The method of claim 2, wherein analysis comprises expression analysis.

8. The method of claim 7, wherein expression analysis comprises quantitative mRNA level analysis.

9. (canceled)

10. The method of claim 7, wherein expression analysis comprises protein analysis.

11. (canceled)

12. The method of claim 2, wherein analysis comprises structural analysis.

13. The method of claim 12, wherein structural analysis comprises copy number variation analysis, FISH analysis or SNP analysis.

14. The method of claim 1, further comprising screening for mutated or low/undetectable levels of CDK1.

15. The method of claim 1, further comprising screening for mutated or low/undetectable levels of Artemis.

16. The method of claim 1, wherein said patient is treated only with PARP1 inhibitor.

17. The method of claim 1, wherein said patient is treated only with ionizing radiation.

18. The method of claim 1, wherein said patient is treated with PARP1 inhibitor and ionizing radiation.

19. The method of claim 1, wherein said patient is a human patient.

20. The method of claim 1, wherein said patient is a non-human mammalian patient.

21. A method of predicting a cancer patient's response to cancer therapy comprising determining expression of Kub-5-Hera (K-H), K-H gene copy number, mutation in a K-H RPR (CID) domain, and/or mutation in K-H coiled-coil domain in cancer cells from said patient, wherein (a) overexpression or increased copy number of K-H confers resistance to PARP1 inhibition, radiation therapy, and chemotherapies that induce double-strand breaks; (b) under-expression of K-H, reduced copy number of K-H, and loss/reduction of function mutation in a K-H RPR (CID) domain, and/or loss/reduction of function mutation in K-H coiled-coil domain in cancer cells from said patient confers sensitivity to PARP1 inhibition, radiation therapy, and chemotherapies that induce double-strand breaks.

22-30. (canceled)

31. A method of predicting a subject's carcinogenic risk comprising determining expression of low/undetectable levels of Kub-5-Hera (K-H), reduced K-H gene copy number, mutation in a K-H RPR (CID) domain, and/or mutation in K-H coiled-coil domain, wherein underexpression, reduced copy number or loss/reduction of function mutation increases risk of carcinogenesis from environmental carcinogens or disease that cause inflammation.

32-45. (canceled)

46. A method of predicting a subject's metastatic potential comprising determining expression levels of Kub-5-Hera (K-H), or K-H gene copy number, wherein (i) overexpression or increased copy number leads to growth changes and enhanced metastic spreading potential.


SEQUENCE LISTING (21 sequences)

SEQ ID NO: 1 - DNA, 22 bp, Artificial
  Synthetic nucleic acid
ttagcgcggtgagtttgaaact

SEQ ID NO: 2 - DNA, 22 bp, Artificial
  Synthetic nucleic acid
ttagcgctgtgagtttgaaact

SEQ ID NO: 3 - DNA, 22 bp, Artificial
  Synthetic nucleic acid
ttagctcggtgagtttgaaact

SEQ ID NO: 4 - DNA, 22 bp, Artificial
  Synthetic nucleic acid
ttagcgcggtgagttttaaact

SEQ ID NO: 5 - DNA, 22 bp, Artificial
  Synthetic nucleic acid
ttagcgcggtgagtggtccact

SEQ ID NO: 6 - RNA, 21 nt, Artificial
  Synthetic primer
gaccguuagguacagaagauu

SEQ ID NO: 7 - RNA, 21 nt, Artificial
  Synthetic primer
ggcugaaacccaaacuauauu

SEQ ID NO: 8 - RNA, 19 nt, Artificial
  Synthetic primer
gcccaugggcuaucaacuu

SEQ ID NO: 9 - RNA, 19 nt, Artificial
  Synthetic primer
guuaagcagugccacacua

SEQ ID NO: 10 - RNA, 19 nt, Artificial
  Synthetic primer
ggaucugaauuuauuauug

SEQ ID NO: 11 - RNA, 19 nt, Artificial
  Synthetic primer
ggaauaggaguacugguuu

SEQ ID NO: 12 - DNA, 59 bp, Artificial
  Synthetic primer
ccgggcaagatgtttctctattggactcgagtccaatagagaaacatcttgcttttttg

SEQ ID NO: 13 - DNA, 57 bp, Artificial
  Synthetic primer
ccgggcgcgatagcgctaataatttctcgagaaattattagcgctatcgcgcttttt

SEQ ID NO: 14 - DNA, 20 bp, Artificial
  Synthetic primer
ctcaatctcgctctcgctct

SEQ ID NO: 15 - DNA, 20 bp, Artificial
  Synthetic primer
ctcgagccataaaaggcaac

SEQ ID NO: 16 - DNA, 20 bp, Artificial
  Synthetic primer
cctctttctttcgcgctcta

SEQ ID NO: 17 - DNA, 20 bp, Artificial
  Synthetic primer
ggaccccgttcctcaatact

SEQ ID NO: 18 - DNA, 20 bp, Artificial
  Synthetic primer
tttttctctagccgcccttt

SEQ ID NO: 19 - DNA, 20 bp, Artificial
  Synthetic primer
ctctccgctcaatttccaag

SEQ ID NO: 20 - DNA, 49 bp, Artificial
  Synthetic primer
gaaggctgtaaaaaacctttagaagcattgctgaacatctggcaagaac

SEQ ID NO: 21 - DNA, 49 bp, Artificial
  Synthetic primer
gttcttgccagatgttcagcaatgcttctaaaggttttttacagccttc